(19) United States
(12) Patent Application Publication
(10) Pub. No.: US 2026/0324639 A1
(43) Pub. Date: Oct. 22, 2026

(54) MICROBIOCIDAL PYRAZOLE DERIVATIVES

(71) Applicant: SYNGENTA CROP PROTECTION AG, Basel (CH)

(72) Inventors: Christopher Charles SCARBOROUGH, Stein (CH); Andrew EDMUNDS, Stein (CH); Atul MAHAJAN, Corlim, Ilhas (IN); Dighe SHASHIKANT, Corlim, Ilhas (IN); Myriem EL QACEMI, Stein (CH); Daniel STIERLI, Stein (CH); Aurelien BIGOT, Stein (CH); Clemens LAMBERTH, Stein (CH)

(73) Assignee: SYNGENTA CROP PROTECTION AG, Basel (CH)

(21) Appl. No.: 19/116,593

(22) PCT Filed: Sep. 29, 2023
(86) PCT No.: PCT/EP2023/077101

§ 371 Date: Mar. 28, 2025

(30) Foreign Application Priority Data
Sep. 30, 2022 (IN) 202211056317
Nov. 18, 2022 (EP) 22208403.0

(51) Int. Cl. A01N 43/56; A01N 43/647; A01N 43/713; A01N 43/80; A01P 3/00; C07D 401/14; C07D 413/14

(52) U.S. Cl. CPC A01N 43/56; A01N 43/647; A01N 43/713; A01N 43/80; A01P 3/00; C07D 401/14; C07D 413/14

(57) ABSTRACT

A compound of formula (I) wherein the substituents are as defined in claim 1, and the agrochemically acceptable salts, stereoisomers, enantiomers, tautomers and N-oxides of those compounds, which can be used as fungicides.

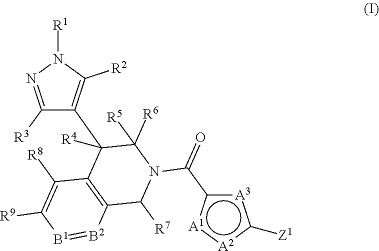

The present invention relates to microbiocidal pyrazole derivatives, e.g., as active ingredients, which have microbiocidal activity, in particular fungicidal activity. The invention also relates to preparation of these pyrazole derivatives, to intermediates useful in the preparation of these pyrazole derivatives, to the preparation of these intermediates, to agrochemical compositions which comprise at least one of the pyrazole derivatives, to preparation of these compositions and to the use of the pyrazole derivatives or compositions in agriculture or horticulture for controlling or preventing infestation of plants, harvested food crops, seeds or non-living materials by phytopathogenic microorganisms, in particular fungi.

According to a first aspect of the present invention, there is provided a compound of formula (I):

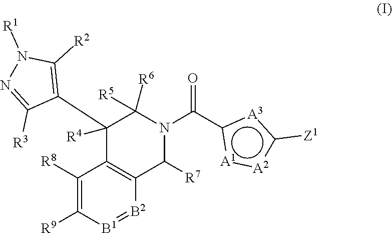

wherein

R¹ is selected from hydrogen, C₁-C₄-alkyl, C₂-C₄-alkenyl, C₂-C₄-alkynyl, or C₃-C₆cycloalkyl;
R² is selected from hydrogen, halogen, C₁-C₄-alkyl, C₂-C₄-alkenyl, C₂-C₄-alkynyl, C₁-C₄-haloalkyl, C₃-C₆cycloalkyl, C₁-C₄-alkylcarbonyl, N—C₁-C₄alkoxy-C-C₁-C₄alkyl-carbonimidoyl, N-hydroxy-C-C₁-C₄alkyl-carbonimidoyl, or C₁-C₄-alkoxycarbonyl;
R³ and R⁴ are independently selected from hydrogen, halogen, or C₁-C₄-alkyl;
R⁵ and R⁶ are independently selected from hydrogen, or C₁-C₄alkyl;
R⁷ is selected from hydrogen, C₁-C₄alkyl, C₁-C₄ alkylcarbonyl, N—C₁-C₄ alkoxy-C-C₁-C₄ alkyl-carbonimidoyl, N-hydroxy-C-C₁-C₄ alkyl-carbonimidoyl, C₁-C₄alkoxycarbonyl, N-methoxy-N-methyl-carbonyl, C₁-C₄ alkylaminocarbonyl, di(C₁-C₆ alkylamino) carbonyl, phenyl, a 5- to 6-membered heteroaryl, or C₃-C₆-cycloalkyl; wherein said 5- or 6-membered heteroaryl comprises 1, 2, 3, or 4 heteroatoms individually selected from N, O, or S and wherein said phenyl and 5- to 6-membered heteroaryl are unsubstituted or substituted with 1, 2, or 3 substituents independently selected from halogen, C₁-C₄ haloalkyl, cyano, carboxy, C₁-C₄ alkyl, or C₁-C₄ alkoxy; and wherein said C₃-C₆-cycloalkyl is unsubstituted or substituted with 1, 2, or 3 substituents independently selected from halogen, C₁-C₄ haloalkyl, cyano, C₁-C₄ alkyl, or C₁-C₄ alkoxy;
B¹ is selected from CR¹⁰, or N;
B² is selected from CR¹¹, or N;
R⁸, R⁹, R¹⁰ and R¹¹ are independently selected from hydrogen, halogen, C₁-C₄alkyl, C₁-C₄haloalkyl, C₁-C₄alkoxy, C₁-C₄ haloalkoxy, C₂-C₄alkenyloxy, C₂-C₄alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-4alkylamino, N,N-diC₁₋₄alkylamino, C₁-C₆ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl, wherein said 5- or 6-membered heteroaryl comprises 1, 2, 3, or 4 heteroatoms individually selected from N, O, or S, and wherein any of said phenyl, 5- to 6-membered heteroaryl and C₃-C₆-cycloalkyl are unsubstituted or substituted by 1, 2, or 3 substituents independently selected from halogen, cyano, C₁-C₄alkyl, C₁-C₄haloalkyl, or C₁-C₄alkoxy;
A¹ is selected from CR^{12a}, or N;
A² is selected from CR^{13a}, or N;
A³ is selected from CR^{14a}, or N;
R^{12a}, R^{13a} and R^{14a} are independently selected from hydrogen, halogen, C₁-C₄alkyl, C₁-C₄haloalkyl, C₂-C₄alkenyl, or C₂-C₄alkynyl; and
Z¹ is selected from C₁-C₄ alkyl, phenyl, 5- or 6-membered heteroaryl, or C₃-C₆ cycloalkyl, wherein said 5- or 6-membered heteroaryl comprises 1, 2, 3, or 4 heteroatoms individually selected from N, O, or S, and wherein any of said phenyl, 5- or 6-membered heteroaryl are unsubstituted or substituted by 1, 2, or 3 substituents independently selected from halogen, cyano, C₁-C₄ alkyl, C₂-C₄ alkenyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl, or C₂-C₄ alkynyl and wherein said C₃-C₆ cycloalkyl is unsubstituted or substituted by 1, 2, or 3 substituents independently selected from halogen, cyano, C₁-C₄ alkyl, C₁-C₄ haloalkyl, or C₁-C₄alkoxy;
or an agrochemically acceptable salt, stereoisomer, or N-oxide thereof.

Surprisingly, it has been found that the compounds of formula (I) have, for practical purposes, a very advantageous level of biological activity for protecting plants against diseases that are caused by fungi.

According to a second aspect of the invention, there is provided an agrochemical composition comprising a fungicidally effective amount of a compound of formula (I) according to the invention. Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of formula (I) according to the invention, or a composition comprising the compound of formula (I), is applied to the plants, to parts thereof or the locus thereof.

According to a fourth aspect of the invention, there is provided the use of a compound of formula (I) according to the invention as a fungicide. According to this particular aspect of the invention, the use may exclude methods for treatment of the human or animal body by surgery or therapy and diagnostic methods practised on the human or animal body.

According to a fifth aspect of the invention, there is provided a compound of formula (IIb), a compound of formula (III) and a compound of formula (IV) as respectively described in the invention.

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄ alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula (I) according to the invention are in free form, in oxidized form as a N-oxide or in salt form, e.g., an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula (I) according to the invention also include hydrates which may be formed during the salt formation.

Where substituents are indicated as being “optionally substituted”, this means that they may or may not carry one or more identical or different substituents, e.g., one, two or three R^{X} substituents. For example, C₁-C₆alkyl substituted by 1, 2 or 3 halogens, may include, but not be limited to, —CH₂Cl, —CHCl₂, —CCl₃, —CH₂F, —CHF₂, —CF₃, —CH₂CF₃, or —CF₂CH₃ groups. As another example, C₁-C₆alkoxy substituted by 1, 2, or 3 halogens, may include, but not be limited to, CH₂ClO—, CHCl₂O—, CClO—, CH₂FO—, CHF₂O—, CF₃O—, CF₃CH₂O—, or CH₃CF₂O-groups. The term “optionally substituted”, as used herein, can be used interchangeably with “unsubstituted or substituted”.

As used herein, the term “halogen” or “halo” refers to fluorine (fluoro), chlorine (chloro), bromine (bromo), or iodine (iodo), preferably fluorine, chlorine, or bromine. This also applies, correspondingly, to halogen in combination with other meanings, such as haloalkyl, haloalkenyl, haloalkynyl, haloalkoxy, and halocycloalkyl.

As used herein, amino means a —NH₂ group.

As used herein, cyano means a —CN group.

As used herein, the term “hydroxyl” or “hydroxy” means an—OH group.

As used herein, the term “carboxylic acid” means a —COOH group.

As used herein, the term “C₁-Cₙ-alkyl” refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2,2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

As used herein, the term “C₂-Cₙ-alkenyl” refers to a straight or branched alkenyl chain moiety having from two to n carbon atoms and one or two double bonds, for example, ethenyl, prop-I-enyl, but-2-enyl.

As used herein, the term “C₂-Cₙ-alkynyl” refers to a straight or branched alkynyl chain moiety having from two to n carbon atoms and one triple bond, for example, ethynyl, prop-2-ynyl, but-3-ynyl,

As used herein, the term “C₃-Cₙ-cycloalkyl” refers to three (3) to n membered cycloalkyl radical such as cyclopropyl, cyclobutyl, cyclopentyl and cyclohexyl.

As used herein, the term “C₁-Cₙ-alkoxy” refers to a straight-chain or branched saturated alkyl radical having one (1) to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy and 1,1-dimethylethoxy. The term “C₂-Cₙ-alkenyloxy” as used herein refers to a straight-chain or branched alkenyl chain having two (2) to n carbon atoms (as mentioned above) which is attached via an oxygen atom.

As used herein, the term “C₁-Cₙ-alkoxy-C₁-Cₙ-alkyl” refers to an alkyl radical (as mentioned above) substituted with a C₁-Cₙ-alkoxy group. Examples are methoxymethyl, methoxyethyl, ethoxymethyl and propoxymethyl.

As used herein, the term “C₁-Cₙ-haloalkyl” refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, or nonafluorobutyl. Accordingly, a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl, or pentafluoroethyl. Similarly, the term “C₂-Cₙ-haloalkenyl” or “C₂-Cₙ-haloalkynyl” as used herein refers to a C₂-Cₙ-alkenyl or C₂-Cₙ-alkynyl radical respectively substituted with one or more halogen atoms which may be the same or different. Similarly, the term “C₃-Cₙ-halocycloalkyl” or “C₁-Cₙ-haloalkoxy” as used herein refers to a C₃-Cₙ-cycloalkyl radical or C₁-Cₙ-alkoxyl radical respectively substituted with one or more halo atoms which may be the same or different.

As used herein, the term “C₁-Cₙ-alkylthio” or “C₁-Cₙ-alkylsulfanyl” refers to a C₁-Cₙ-alkyl group linked through a sulfur atom.

As used herein, the term “C₁-Cₙ-haloalkylthio” or “C₁-Cₙ-haloalkylsulfanyl” refers to a C₁-Cₙhaloalkyl group linked through a sulfur atom.

As used herein, the term “C₁-Cₙ-alkylsulfinyl” refers to a C₁-Cₙalkyl group linked through the sulfur atom of a sulfinyl (or S(═O)—) group.

As used herein, the term “C₁-Cₙ-alkylsulfonyl” refers to a C₁-Cₙalkyl group linked through the sulfur atom of a sulfonyl (or S(═O)₂—) group.

As used herein, the term “C₁-Cₙ-alkylsulfonyl-C₁-Cₙ-alkyl” refers to an a C₁-Cₙalkyl radical substituted with a C₁-Cₙalkylsulfonyl group.

As used herein, the term “C₁-Cₙ-alkylcarbonyl” refers to a C₁-Cₙ-alkyl group linked through the carbon atom of a carbonyl (C═O) group.

As used herein, the term “C₁-Cₙ-alkoxycarbonyl” refers to a C₁-Cₙ-alkoxy moiety linked through a carbon atom of a carbonyl (or C═O) group.

As used herein, the term “benzoyl” refers to a phenyl group linked through the carbon atom of a carbonyl (C═O) group.

As used herein, the term “C₁-Cₙ-alkylaminocarbonyl” refers to a C₁-Cₙ-alkylamino group (or RₐNHC(═O)—, wherein Rₐ is a C₁-Cₙ-alkyl group) linked through the carbon atom of a carbonyl (C═O) group.

As used herein, the term “aminocarbonyl-C₁-Cₙ-alkyl” refers to a C₁-Cₙ-alkyl radical substituted by an aminocarbonyl (or NH₂C(═O)—) group.

As used herein, the term “N—C₁-C₄ alkoxy-C-C₁-C₄ alkyl-carbonimidoyl” refers to a radical of the formula-C(Rₐ)═NO(R_{b}) where Rₐ is a C₁-C₄ alkyl radical as generally defined above, and R_{b} is a C₁-C₄ alkyl radical as generally defined above.

As used herein the term “N-hydroxy-C-C₁-C₄ alkyl-carbonimidoyl” refers to a radical of the formula-C(Rₐ)═NOH where Rₐ is a C₁-C₄ alkyl radical as generally defined above.

As used herein, the term “heteroaryl” refers to a 5- or 6-membered aromatic monocyclic ring radical which comprises 1, 2, 3 or 4 heteroatoms individually selected from N, O or S. Examples of heteroaryl include, but are not limited to, furanyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl. The term “heteroaryl-C₁-Cₙ-alkyl” or “heteroaryl-C₃-Cₙ-cycloalkyl” refers to an C₁-Cₙ-alkyl or C₃-Cₙ-cycloalkyl radical respectively substituted by a heteroaryl group. The heteroaryl-C₁-Cₙ-alkyl or heteroaryl-C₃-Cₙ-cycloalkyl radical may be substituted on heteroaryl, alkyl and/or cycloalkyl group as appropriate.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain, and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, using known techniques and by observing results obtained under analogous circumstances. In determining the effective amount, a number of factors are considered including, but not limited to the type of plant or derived product to be applied; the pest to be controlled & its lifecycle; the particular compound applied; the type of application; and other relevant circumstances.

As used herein, the term “room temperature” or “RT” or “rt” refer to a temperature of about 15° C. to about 35° C. For example, rt can refer to a temperature of about 20° C. to about 30° C.

The following list provides definitions, including preferred definitions, for substituents R¹, R², R³, R⁴, R⁵, R⁶, R⁷, R⁸, R⁹, B¹, B², A¹, A², A³ and Z with reference to the compounds of formula (I) of the present invention. For any one of these substituents, any of the definitions given below may be combined with any definition of any other substituent given below or elsewhere in this document.

In an embodiment of the invention, R¹ is selected from hydrogen, C₁-C₄-alkyl, C₂-C₄-alkenyl, C₂-C₄-alkynyl, or C₃-C₆cycloalkyl. In another embodiment of the invention, R¹ is selected from hydrogen, C₁-C₄-alkyl, C₂-C₄-alkenyl, C₂-C₄-alkynyl, or C₃-C₆cycloalkyl. Preferably R¹ is C₁-C₄ alkyl. More preferably, R¹ is methyl, ethyl, or isopropyl. Most preferably R¹ is methyl.

In an embodiment of the invention, R² is selected from hydrogen, halogen, C₁-C₄-alkyl, C₂-C₄-alkenyl, C₂-C₄-alkynyl, C₁-C₄-haloalkyl, C₃-C₆cycloalkyl, C₁-C₄-alkylcarbonyl, N—C₁-C₄alkoxy-C-C₁-C₄ alkyl-carbonimidoyl, N-hydroxy-C-C₁-C₄ alkyl-carbonimidoyl, or C₁-C₄ alkoxycarbonyl. In another embodiment of the invention R² is selected from hydrogen, halogen, C₁-C₄-alkyl, C₂-C₄-alkenyl, C₂-C₄-alkynyl, C₁-C₄-haloalkyl, C₃-C₆cycloalkyl, C₁-C₄-alkylcarbonyl, N—C₁-C₄alkoxy-C-C₁-C₄alkyl-carbonimidoyl, N-hydroxy-C-C₁-C₄ alkyl-carbonimidoyl, or C₁-C₄-alkoxycarbonyl. In still another embodiment R² is selected from hydrogen, halogen, C₁-C₄ alkyl, C₃-C₆ cycloalkyl, C₁-C₄ alkylcarbonyl, N—C₁-C₄ alkoxy-C₁-C₄ alkyl-carbonimidoyl, or N-hydroxy-C₁-C₄ alkyl-carbonimidoyl. Preferably, R² is selected from hydrogen, halogen, methyl, ethyl, cyclopropyl, C₁-C₂ alkylcarbonyl, N—C₁-C₂ alkoxy-C₁-C₂ alkyl-carbonimidoyl, or N-hydroxy-C₁-C₂ alkyl-carbonimidoyl. More preferably, R² is selected from hydrogen, fluorine, chlorine, bromine, methyl, ethyl, cyclopropyl, acetyl, —C(CH₃)═NOCH₃, —C(CH₃)═NOCH₂CH₃, or —C(CH₃)═NOH. In a preferred embodiment of the invention R² is selected from hydrogen, halogen, or C₁-C₄ alkyl. Preferably R² is hydrogen, chlorine, or methyl.

In one embodiment R³ is selected from hydrogen, halogen, or C₁-C₄ alkyl. In another embodiment R³ is selected from hydrogen, halogen, or C₁-C₄ alkyl. Preferably R³ is hydrogen or C₁-C₄ alkyl. More preferably R³ is hydrogen or methyl. Most preferably R³ is hydrogen.

In one embodiment of the invention R⁴ is selected from hydrogen, halogen, or C₁-C₄-alkyl. In another embodiment of the invention R⁴ is hydrogen, halogen, or C₁-C₄alkyl. Preferably R⁴ is hydrogen, chlorine, bromine, fluorine, methyl, or ethyl. More preferably R⁴ is hydrogen, chlorine, bromine, or methyl. Still more preferably R⁴ is hydrogen or methyl. In one embodiment of the invention R⁴ is hydrogen. In another embodiment of the invention R⁴ is methyl.

In one embodiment of the invention R⁵ and R⁶ are independently selected from hydrogen, or C₁-C₆alkyl. In another embodiment of the invention R⁵ and R⁶ are independently selected from hydrogen or C₁-C₄-alkyl. Preferably R⁵ and R⁶ are independently selected from hydrogen, methyl, or ethyl. More preferably, R⁵ and R⁶ are independently selected from hydrogen or methyl. Even more preferably, R⁵ and R⁶ are hydrogen.

In an embodiment of the invention R⁷ is selected from hydrogen, C₁-C₄alkyl, C₁-C₄alkylcarbonyl, C₁-C₄ alkoxycarbonyl, N—C₁-C₄ alkoxy-C-C₁-C₄alkyl-carbonimidoyl, N-hydroxy-C-C₁-C₄ alkyl-carbonimidoyl, N-methoxy-N-methyl-carbonyl, C₁-C₄ alkylaminocarbonyl, di(C₁-C₆ alkylamino) carbonyl, phenyl, a 5- to 6-membered heteroaryl, or C₃-C₆-cycloalkyl, wherein said 5- to 6-membered heteroaryl comprises 1 heteroatom selected from N and wherein said phenyl and 5- to 6-membered heteroaryl are unsubstituted or substituted with 1 or 2 substituent independently selected from halogen, C₁-C₄haloalkyl, cyano, or C₁-C₄alkyl; and wherein said C₃-C₆-cycloalkyl is unsubstituted or substituted with 1 substituent selected from cyano. Preferably R⁷ is selected from hydrogen, methyl, acetyl, C(CH₃)═NOCH₃, —C(CH₃)═NOCH₂CH₃, —C(CH₃)═NOH, methoxycarbonyl, ethoxycarbonyl, N-methoxy-N-methyl-carbonyl, methylaminocarbonyl, dimethylaminocarbonyl, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (5-fluoropyrazol-1-yl), (3,5-dimethylpyrazol-1-yl), (5-methylpyrazol-1-yl), pyrazol-1-yl, cyclopropyl, or 1-cyanocyclopropyl. More preferably R⁷ is selected from hydrogen, methyl, acetyl, C(CH₃)═NOCH₃, —C(CH₃)═NOCH₂CH₃, —C(CH₃)═NOH, phenyl, 4-cyanophenyl, pyrazol-1-yl, cyclopropyl, or 1-cyanocyclopropyl. Even more preferably R⁷ is selected from hydrogen, methyl, cyclopropyl, or 1-cyanocyclopropyl.

In another embodiment R⁷ is selected from hydrogen, C₁-C₄ alkyl, or C₃-C₆-cycloalkyl. Preferably R⁷ is hydrogen, methyl, cyclopropyl, or 1-cyanocyclopropyl. Even more preferably R⁷ is hydrogen, methyl, or cyclopropyl. In one embodiment R⁷ is hydrogen or methyl. In another embodiment R⁷ is hydrogen. In still another embodiment R⁷ is methyl.

In an embodiment of the invention, R⁸ and R⁹ are independently selected from hydrogen, halogen, cyano, C₁-C₄ alkyl, or C₁-C₄ alkoxy. Preferably, R⁸ and R⁹ are independently selected from hydrogen, halogen, methyl, methoxy, or cyano. More preferably, R⁸ and R⁹ are independently selected from hydrogen, methyl, chlorine, fluorine, bromine, or methoxy. Even more preferably, R⁸ and R⁹ are hydrogen or methoxy,

In another embodiment of the invention, R⁸ is selected from hydrogen, halogen, or cyano. More preferably, R⁸ is hydrogen, cyano, or bromine. Even more preferably, R⁸ is hydrogen.

In another embodiment R⁹ is selected from hydrogen, halogen, C₁-C₃ alkyl, C₁-C₂ haloalkyl, C₁-C₃ haloalkoxy, C₁-C₄alkoxy, C₁-C₅alkenyloxy, C₁-C₃ alkynyloxy, C₁-C₂ alkylsulfanyl, C₁-C₂ alkylsulfinyl, C₁-C₂ alkylsulfonyl, C₁-C₂ alkoxy-C₁-C₂ alkyl, C₁-C₃ alkoxycarbonyl, C₁-C₂ alkylcarbonyl, N—C₁-C₂ alkoxy-C-C₁-C₂ alkyl-carbonimidoyl, N-hydroxy-C-C₁-C₂ alkyl-carbonimidoyl hydroxy, C₁-C₂ alkylaminocarbonyl, di(C₁-C₂ alkylamino) carbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl, or 1-cyanocyclopropyl. Preferably R⁹ is selected from hydrogen, chloro, fluoro, bromo, methyl, ethyl, trifluoromethyl, difluoromethyl, difluoromethoxy, 2,2-difluoroethoxy, 2,2,2-trifluoroethoxy, methoxy, ethoxy, propoxy, -allyloxy, prop-2-ynoxy, methylsulfanyl, methylsulfinyl, methylsulfonyl, methoxymethyl, ethoxymethyl, 2-methoxyethoxymethyl, methoxycarbonyl, ethoxycarbonyl, tert-butoxycarbonyl, acetyl, propanoyl, —C(CH₃)═NOCH₃, —C(CH₃)═NOCH₂CH₃, —C(CH₃)═NOH, methylaminocarbonyl, di(methylamino) carbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl, or 1-cyanocyclopropyl. More preferably R⁹ is selected from hydrogen, chloro, bromo, fluoro, methyl, methoxy, propoxy, allyloxy, methoxymethyl, 2-methoxyethoxymethyl, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl, or 1-cyanocyclopropyl. Even more preferably R⁹ is hydrogen, chloro, fluoro, methyl,, or methoxy. Most preferably R⁹ is hydrogen or methoxy.

In an embodiment of the invention, B¹ is CR¹⁰ and B² is CR¹¹, or B¹ is N and B² is CR¹¹, or B¹ is CR¹⁰ and B² is N. Preferably, B¹ is CR¹⁰ and B² is CR¹¹.

In one embodiment of the invention R¹⁰ and R¹¹ are independently selected from hydrogen, halogen, hydroxy, cyano, amino, C₁-C₄ alkyl, C₁-C₄ haloalkyl, C₁-C₄ alkoxy, C₁-C₄ haloalkoxy, C₂-C₄alkenyloxy, C₂-C₄ alkynyloxy, C₁-C₄alkylsulfanyl, C₁-C₄ alkylsulfinyl, C₁-C₄ alkylsulfonyl, C₁-C₄ alkoxy-C₁-C₄ alkyl, N—C₁-4alkylamino, N, N-diC₁₋₄ alkylamino, C₁-C₆ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, carboxy, phenyl, 5- or 6-membered heteroaryl, or C₃-C₆ cycloalkyl, wherein said 5- or 6-membered heteroaryl comprises 1, 2, 3, or 4 heteroatoms individually selected from N, O, or S, and wherein any of said phenyl, 5- or 6-membered heteroaryl and C₃-C₆-cycloalkyl are unsubstituted or substituted by 1, 2, or 3 substituents independently selected from halogen, cyano, C₁-C₄ alkyl, C₁-C₄haloalkyl, or C₁-C₄ alkoxy. Preferably R¹⁰ and R¹¹ are independently selected from hydrogen, chloro, fluoro, bromo, methyl, ethyl, trifluoromethyl, difluoromethyl, difluoromethoxy, 2,2-difluoroethoxy, 2,2,2-trifluoroethoxy, methoxy, ethoxy, propoxy, -allyloxy, prop-2-ynoxy, methylsulfanyl, methylsulfinyl, methylsulfonyl, methoxymethyl, ethoxymethyl, 2-methoxyethoxymethyl, methoxycarbonyl, ethoxycarbonyl, tert-butoxycarbonyl, acetyl, propanoyl, —C(CH₃)═NOCH₃, —C(CH₃)═NOCH₂CH₃, —C(CH₃)═NOH, methylaminocarbonyl, di(methylamino) carbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, cyclopropyl, or 1-cyanocyclopropyl. More preferably R¹⁰ and R¹¹ are independently selected from hydrogen, chloro, bromo, fluoro, methyl, methoxy, propoxy, allyloxy, methoxymethyl, 2-methoxyethoxymethyl, phenyl, 2-cyanophenyl, 3-cyanophenyl, 4-cyanophenyl, [4-(trifluoromethyl)pyrazol-1-yl], [3-(trifluoromethyl)pyrazol-1-yl], (3-cyanopyrazol-1-yl), (4-cyanopyrazol-1-yl), (5-chloropyrazol-1-yl), (4-chloropyrazol-1-yl), (3-chloropyrazol-1-yl), (5-fluoropyrazol-1-yl), (4-fluoropyrazol-1-yl), (3-fluoropyrazol-1-yl), (3,5-dimethylpyrazol-1-yl), (5-methylpyrazol-1-yl), (4-methylpyrazol-1-yl), (3-methylpyrazol-1-yl), pyrazol-1-yl, C(CH₃)═NOCH₃, —C(CH₃)═NOCH₂CH₃, —C(CH₃)═NOH, cyclopropyl, or 1-cyanocyclopropyl.

In another embodiment R¹⁰ and R¹¹ are independently selected from hydrogen, halogen, C₁-C₃ alkyl, or C₁-C₄ alkoxy. Preferably R¹⁰ and R¹¹ are independently selected from hydrogen, or halogen. More preferably R¹⁰ and R¹¹ are hydrogen.

In an embodiment of the invention A¹ is CR^{12a}, or N; A² is CR^{13a}, or N; A³ is CR^{14a}, or N. Preferably at least two of A¹, A² and A³ are selected from N. More preferably A¹, A² and A³ are N.

In an embodiment of the invention R^{12a}, R^{13a} and R^{14a} are independently selected from hydrogen, halogen, C₁-C₄ alkyl, C₃-C₆-cycloalkyl, or C₁-C₄ haloalkyl. Preferably R^{12a}, R^{13a} and R^{14a} are independently selected from hydrogen, halogen, methyl, cyclopropyl, or trifluoromethyl. More preferably R^{12a}, R^{13a} and R^{14a} are hydrogen. In another embodiment A¹ is selected from CR^{12a}, or N; A² is selected from CR^{13a}, or N; A³ is selected from CR^{14a}, or N; wherein at least two of A¹, A² and A³ are selected from N; and wherein R^{12a}, R^{13a}, R^{14a} are hydrogen.

In an embodiment of the invention Z¹ is selected from C₁-C₄ alkyl, phenyl, a 5- to 6-membered heteroaryl, or C₃-C₆-cycloalkyl; wherein said 5- or 6-membered heteroaryl comprises 1, 2, 3, or 4 heteroatoms individually selected from N, O, or S, and wherein any of said phenyl and 5- to 6-membered heteroaryl are unsubstituted or substituted with 1, 2, or 3 substituents independently selected from halogen, cyano, C₁-C₄ alkyl, C₁-C₄ haloalkyl, C₁-C₄ haloalkoxy, C₁-C₄ alkoxy, C₂-C₄-alkynyl, C₁-C₄ alkylsulfanyl, C₁-C₄ alkylsulfinyl, or C₁-C₄ alkylsulfonyl and wherein said C₃-C₆-cycloalkyl is unsubstituted or substituted with 1, 2, or 3 substituents independently selected from halogen, cyano, C₁-C₄ alkyl, C₁-C₄ haloalkyl, or C₁-C₄ alkoxy.

In another embodiment Z¹ is selected from C₁-C₃ alkyl, phenyl, a 5- to 6-membered heteroaryl, or C₃-C₆-cycloalkyl; wherein the 5- or 6-membered heteroaryl comprises 1, 2, or 3 heteroatoms individually selected from N, O, or S, and wherein any of said phenyl and 5- to 6-membered heteroaryl are unsubstituted or substituted with 1, 2, or 3 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another embodiment Z¹ is selected from C₁-C₃ alkyl, phenyl, a 5- to 6-membered heteroaryl, or C₃-C₆-cycloalkyl, wherein said 5- or 6-membered heteroaryl comprises 1 heteroatom selected from N, O, or S, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl. Preferably Z¹ is selected from 1-methylpyrazol-4-yl, 2,3,4-trifluorophenyl, 2,3-difluorophenyl, 3,4-difluorophenyl, 2,4,6-trifluorophenyl, 2,4-difluorophenyl, 2,5-difluorophenyl, 3,5-difluoro-2-pyridyl, 5-fluoro-2-pyridyl, 3-fluoro-2-pyridyl, 2-fluoro-4-methoxy-phenyl, 2-fluoro-4-methylsulfonyl-phenyl, 2-fluorophenyl, 3-fluorophenyl, 4-fluorophenyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-methylphenyl, 3-methylphenyl, 4-methylphenyl, 2-chlorophenyl, 3-chlorophenyl, 4-chlorophenyl, 3-methoxyphenyl, 4-ethynyl-2-fluoro-phenyl, 4-fluoro-2-methoxy-phenyl, cyclopropyl, 1-methylcyclopropyl, cyclobutyl, cyclohexyl, cyclopentyl, methyl, n-propyl, or phenyl. More preferably Z¹ is selected from 1-methylpyrazol-4-yl, 2,4,6-trifluorophenyl, 2,4-difluorophenyl, 2,5-difluorophenyl, 2-chlorophenyl, 2-fluorophenyl, 3,5-difluoro-2-pyridyl, 2-furyl, 2-methylphenyl, 2-thienyl, 3,4-difluorophenyl, 3-chlorophenyl, 3-fluorophenyl, 3-methylphenyl, 3-thienyl, 4-fluoro-2-methoxy-phenyl, 4-fluorophenyl, 4-methylphenyl, cyclobutyl, cyclohexyl, cyclopentyl, methyl, or phenyl. Even more preferably Z¹ is selected from 1-methylpyrazol-4-yl, 2,4,6-trifluorophenyl, 3,5-difluoro-2-pyridyl, 2,4-difluorophenyl, 2-fluorophenyl, 2-furyl, 2-methylphenyl, 2-thienyl, 3,4-difluorophenyl, 3-chlorophenyl, 3-thienyl, 4-fluoro-2-methoxy-phenyl, 4-fluorophenyl, cyclobutyl, cyclohexyl, cyclopentyl, or phenyl. Still even more preferably Z¹ is selected from 2,4-difluorophenyl, 3,5-difluoro-2-pyridyl, 2-fluorophenyl, 4-fluorophenyl, or phenyl.

In another embodiment of the invention Z¹ is selected from C₁-C₄ alkyl, phenyl, a 5- to 6-membered heteroaryl, or C₃-C₆-cycloalkyl; wherein said 5- or 6-membered heteroaryl comprises 1, 2 or 3 heteroatoms individually selected from N, O, or S, and wherein said phenyl and 5- to 6-membered heteroaryl are unsubstituted or substituted with 1 or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen or C₁-C₄ alkyl. Preferably Z¹ is selected from phenyl or a 5- to 6-membered heteroaryl; wherein said 5- or 6-membered heteroaryl comprises 1 heteroatom selected from N or S, and wherein said phenyl and -5 to 6-membered heteroaryl are unsubstituted or substituted with 1 or 2 substituents independently selected from fluoro, chloro, C₁-C₄alkyl, or C₁-C₄alkoxy. More preferably Z¹ is selected from 1-methylpyrazol-4-yl, 2,4,6-trifluorophenyl, 2,4-difluorophenyl, 3,5-difluoro-2-pyridyl, 5-fluoro-2-pyridyl, 3-fluoro-2-pyridyl, 2-fluorophenyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-methylphenyl, 2-thienyl, 3,4-difluorophenyl, 3-chlorophenyl, 3-thienyl, 4-fluoro-2-methoxy-phenyl, 4-fluorophenyl, cyclobutyl, cyclohexyl, cyclopentyl, or methyl. Still more preferably Z¹ is selected from 2,4-difluorophenyl, 3,5-difluoro-2-pyridyl, 5-fluoro-2-pyridyl, 3-fluoro-2-pyridyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-fluorophenyl, 4-fluorophenyl, or phenyl.

In another embodiment of the invention Z¹ is selected from phenyl, a 5- to 6-membered heteroaryl, or C₃-C₆-cycloalkyl; wherein said 5- or 6-membered heteroaryl comprises 1 heteroatom selected from N or S, and wherein said phenyl and 5- to 6-membered heteroaryl are unsubstituted or substituted with 1 or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen, or C₁-C₄ alkyl. Preferably Z¹ is selected from phenyl, or a 5- to 6-membered heteroaryl; wherein said 5- or 6-membered heteroaryl comprises 1 heteroatom selected from N or S, and wherein said phenyl and 5- to 6-membered heteroaryl are unsubstituted or substituted with 1 or 2 substituents independently selected from fluoro, chloro, C₁-C₄ alkyl, or C₁-C₄ alkoxy.

The present invention, accordingly, makes available a compound of formula (I) having R¹, R², R³, R⁴, R⁵, R⁶, R⁷, R⁸, R⁹, B¹, B², A¹, A², A³ and Z¹ as defined above in all combinations/each permutation.

Embodiments according to the invention are provided as set out below.

In one embodiment of the invention, there is provided a compound of formula (I) wherein
R¹ is selected from hydrogen, C₁-C₆-alkyl, C₂-C₆-alkenyl, C₂-C₆-alkynyl, or C₃-C₆-cycloalkyl;
R² is selected from hydrogen, halogen, C₁-C₆-alkyl, C₂-C₆-alkenyl, C₂-C₆-alkynyl, C₁-C₆-haloalkyl, C₃-C₆cycloalkyl, C₁-C₄-alkylcarbonyl, N—C₁-C₄ alkoxy-C-C₁-C₄ alkyl-carbonimidoyl, N-hydroxy-C-C₁-C₄ alkyl-carbonimidoyl, or C₁-C₆-alkoxycarbonyl;
R³ and R⁴ are independently selected from hydrogen, halogen, or C₁-C₆-alkyl;
R⁵ and R⁶ are independently selected from hydrogen, or C₁-C₆alkyl;
R⁷ is selected from hydrogen, C₁-C₆alkyl, C₁-C₆ alkylcarbonyl, N—C₁-C₄ alkoxy-C-C₁-C₄ alkyl-carbonimidoyl, N-hydroxy-C-C₁-C₄ alkyl-carbonimidoyl, C₁-C₆ alkoxycarbonyl, N-methoxy-N-methyl-carbonyl, C₁-C₆ alkylaminocarbonyl, di(C₁-C₆ alkylamino) carbonyl, phenyl, a 5- to 6-membered heteroaryl and C₃-C₆-cycloalkyl; wherein the 5- or 6-membered heteroaryl comprises 1, 2, 3, or 4 heteroatoms individually selected from N, O, or S, and wherein said phenyl and 5- to 6-membered heteroaryl are unsubstituted or substituted with 1, 2, or 3 substituents independently selected from halogen, C₁-C₄ haloalkyl, cyano, carboxy, C₁-C₄ alkyl and C₁-C₄ alkoxy; and wherein said C₃-C₆-cycloalkyl is unsubstituted or substituted with 1, 2, or 3 substituents independently selected from halogen, C₁-C₄ haloalkyl, cyano, C₁-C₄ alkyl and C₁-C₄ alkoxy; B¹ is selected from CR¹⁰, and N;
B² is selected from CR¹¹, and N;
R⁸, R⁹, R¹⁰ and R¹¹ are independently selected from hydrogen, halogen, C₁-C₄ alkyl, C₁-C₄ haloalkyl, C₁-C₄ alkoxy, C₁-C₄ haloalkoxy, C₂-C₄alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-4alkylamino, N,N-diC₁₋₄alkylamino, C₁-C₆ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl, wherein the 5- or 6-membered heteroaryl comprises 1, 2, 3, or 4 heteroatoms individually selected from N, O, or S, and wherein any of said phenyl, 5- or 6-membered heteroaryl and C₃-C₆-cycloalkyl are unsubstituted or substituted by 1, 2, or 3 substituents independently selected from halogen, cyano, C₁-C₄ alkyl, C₁-C₄ haloalkyl, or C₁-C₄alkoxy;
A¹ is selected from CR^{12a}, or N;
A² is selected from CR^{13a}, or N;
A³ is selected from CR^{14a}, or N;
R^{12a}, R^{13a} and R^{14a} are independently selected from hydrogen, halogen, C₁-C₄ alkyl, C₁-C₄haloalkyl, C₂-C₄ alkenyl, or C₂-C₄ alkynyl; and
Z¹ is selected from C₁-C₄ alkyl, phenyl, 5- or 6-membered heteroaryl, or C₃-C₆ cycloalkyl, wherein the 5- or 6-membered heteroaryl comprises 1, 2, 3, or 4 heteroatoms individually selected from N, O, or S, and wherein any of said phenyl, 5- or 6-membered heteroaryl are unsubstituted or substituted by 1, 2, or 3 substituents independently selected from halogen, cyano, C₁-C₄ alkyl, C₂-C₄ alkenyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl and C₂-C₄ alkynyl and wherein said and C₃-C₆ cycloalkyl are unsubstituted or substituted by 1, 2, or 3 substituents independently selected from halogen, cyano, C₁-C₄ alkyl, C₁-C₄ haloalkyl, C₁-C₄ alkoxy;
or an agrochemically acceptable salt, stereoisomer, or N-oxide thereof.

In one embodiment, in a compound of formula (I) according to the present invention, wherein
R¹ is hydrogen, C₁-C₄ alkyl, C₂-C₄alkenyl, C₂-C₄ alkynyl, or C₃-C₆cycloalkyl;
R² is hydrogen, halogen, or C₁-C₄ alkyl;
R³ and R⁴ are independently selected from hydrogen, halogen, or C₁-C₄ alkyl;
R⁵ and R⁶ are independently selected from hydrogen and C₁-C₄ alkyl;
R⁷ is hydrogen, C₁-C₄alkyl, or C₃-C₆-cycloalkyl, wherein said C₃-C₆-cycloalkyl is unsubstituted or substituted with 1 substituent selected from halogen, C₁-C₄ haloalkyl, cyano, C₁-C₄ alkyl, or C₁-C₄ alkoxy;
B¹ is selected from CR¹⁰, and N;
B² is selected from CR¹¹, and N;
R⁸ and R⁹ are independently selected from hydrogen, halogen, cyano, C₁-C₄ alkyl, or C₁-C₄ alkoxy;
R¹⁰ and R¹¹ are independently selected from hydrogen, halogen, C₁-C₃ alkyl, or C₁-C₄ alkoxy;
A¹ is selected from CR^{12a}, or N;
A² is selected from CR^{13a}, or N;
A³ is selected from CR^{14a}, or N;
R^{12a}, R^{13a} and R^{14a} are independently selected from hydrogen, halogen, C₁-C₄ alkyl, C₃-C₆-cycloalkyl, or C₁-C₄haloalkyl; and
Z¹ is phenyl, a 5- to 6-membered heteroaryl, or C₃-C₆-cycloalkyl, wherein said 5- or 6-membered heteroaryl comprises 1 heteroatom selected from N and S, and wherein said phenyl and 5- to 6-membered heteroaryl are unsubstituted or substituted with 1, or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen, or C₁-C₄ alkyl.

In an embodiment of the invention, the compound of formula (I) may be a compound of formula (II):

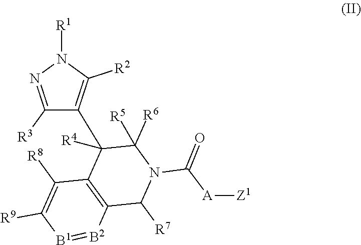

wherein
R¹, R², R³, R⁴, R⁵, R⁶, R⁷, R⁸, R⁹, B¹ and B² and Z¹ are as defined for the compounds of formula (I) according to the present invention, and
A is selected from:

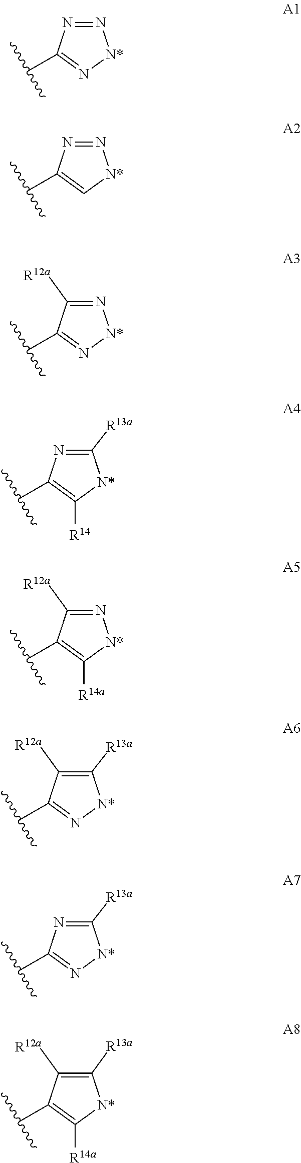

wherein

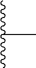

indicates the bond to the C(═O) group and the star (*) the nitrogen with the bond to the Z¹ group and wherein R^{12a}, R^{13a} and R^{14a} are independently selected from hydrogen, halogen, C₁-C₄ alkyl, C₃-C₆ cycloalkyl, or C₁-C₄ haloalkyl.

In one embodiment of the invention R^{12a}, R^{13a} and R^{14a} are independently selected from hydrogen, halogen, methyl, cyclopropyl, or trifluoromethyl.

In another embodiment of the invention R^{12a}, R^{13a} and R^{14a} are hydrogen.

Preferably, in the compound of formula (II) R¹, R², R³, R⁴, R⁵, R⁶, R⁷, R⁸, R⁹ and Z¹ are as defined for the compounds of formula (I) according to the present invention. A is selected from A¹, A², A³, A⁴, A⁵, A⁶, A⁷, or A⁸; R¹², R¹³ and R¹⁴ are hydrogen; B¹ is CR¹⁰; and B² is CR¹¹; wherein R¹⁰ and R¹¹ are as defined for the compounds of formula (I) according to the present invention.

More preferably, in the compound of formula (II) R¹, R², R³, R⁴, R⁵, R⁶, R⁷, R⁸, R⁹ and Z¹ are as defined for the compounds of formula (I) according to the present invention, A is selected from A1, A2, A3, A4, A5, A6, A7, or A8; R¹², R¹³ and R¹⁴ are hydrogen; B¹ is CH and B² is CH.

In an embodiment of the invention, in the compound of formula (II) A is selected from:

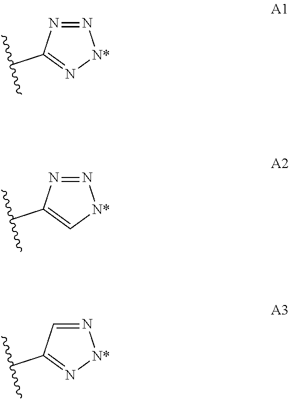

wherein

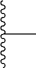

indicates the bond to the C(═O) group and the star (*) the Nitrogen with the bond to the Z¹ group.

In a preferred embodiment of the invention, in the compound of formula (II) A is selected from

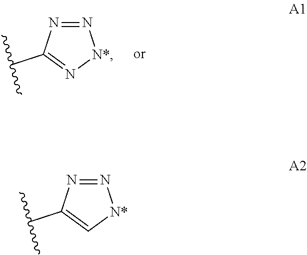

wherein

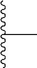

indicates the bond to the C(═O) group and the star (*) the Nitrogen with the bond to the Z¹ group.

Preferably, in the compound of formula (II) R¹, R², R³, R⁴, R⁵, R⁶, R⁷, R⁸, R⁹ and Z¹ are as defined for the compounds of formula (I) according to the present invention, A is selected from A1, A2, or A3; B¹ is CR¹⁰; and
B² is CR¹¹; wherein R¹⁰ and R¹¹ are as defined for the compounds of formula (I) according to the present invention.

More preferably, in the compound of formula (II) R¹, R², R³, R⁴, R⁵, R⁶, R⁷, R⁸, R⁹ and Z¹ are as defined for the compounds of formula (I) according to the present invention, A is selected from A1, A2, or A3; B¹ is CH; and B² is CH.

Even more preferably, in the compound of formula (II) R¹, R², R³, R⁴, R⁵, R⁶, R⁷, R⁸, R⁹ and Z¹ are as defined for the compounds of formula (I) according to the present invention, A is selected from A1, or A2; B¹ is CH; and B² is CH.

In an embodiment of the invention, in the compound of formula (II) A is selected from:

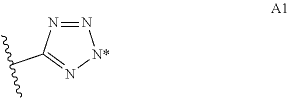

wherein

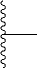

indicates the bond to the C(═O) group, and the star (*) the Nitrogen with the bond to the Z¹ group.

In an embodiment of the invention, the compound of formula (II) may be a compound of formula (II-A) wherein B¹ and B² are CH, and A is defined as for compound of formula (II)

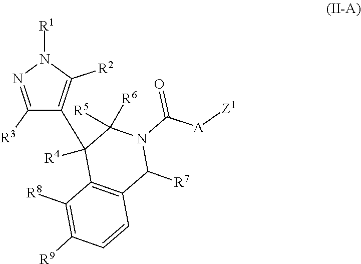

wherein R¹, R², R³, R⁴, R⁵, R⁶, R⁷, R⁸, R⁹ and Z¹ are as defined for the compounds of formula (I) according to the present invention.

In a variant of this embodiment of the invention, the compound of formula (II-A) may be a compound of formula (II-A1) wherein B¹ and B² are CH; R³, R⁵, R⁶ are hydrogen, and A is defined as for compound (II):

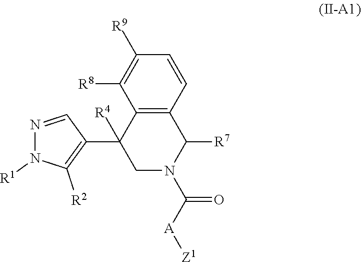

wherein R¹, R², R⁴, R⁷, R⁸, R⁹ and Z¹ are as defined for the compounds of formula (I) according to the present invention.

Preferably in the compound of formula (II-A1) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy;
A is defined as for compound (II), and Z¹ is defined as for the compounds of formula (I) according to the present invention.

Preferably in the compound of formula (II-A1) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy; A is selected from A1, or A2; and
Z¹ is defined as for the compounds of formula (I) according to the present invention.

Preferably in the compound of formula (II-A1) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy;
A is selected from A1, or A2; and
Z¹ is phenyl, a 5- to 6-membered heteroaryl, or C₃-C₆-cycloalkyl, wherein said 5- or 6-membered heteroaryl comprises 1 heteroatom selected from N, or S, and wherein said phenyl and 5- to 6-membered heteroaryl are unsubstituted or substituted with 1, or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen, or C₁-C₄ alkyl.

In an embodiment of the invention, the compound of formula (II) may be a compound of formula (II-B) wherein B¹ and B² are CH, and wherein A is A1,

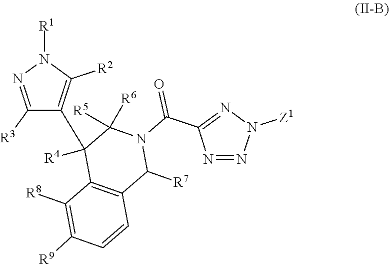

R¹, R², R³, R⁴, R⁵, R⁶, R⁷, R⁸, R⁹, and Z¹ are as defined for the compounds of formula (I) according to the present invention.

In a variant of this embodiment of the invention, the compound of formula (II-B) may be a compound of formula (II-B1) wherein B¹ and B² are CH; R³, R⁵, R⁶ are hydrogen, and A is A1

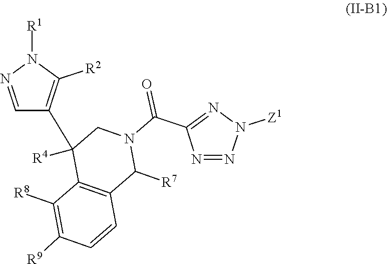

wherein R¹, R², R⁴, R⁷, R⁸, R⁹ and Z¹ are as defined for the compounds of formula (I) according to the present invention.

Preferably in the compound of formula (II-B1) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy; and
Z¹ is defined as for the compounds of formula (I) according to the present invention.

Preferably in the compound of formula (II-B1) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy; and
Z¹ is phenyl, a 5- to 6-membered heteroaryl, or C₃-C₆-cycloalkyl, wherein said 5- or 6-membered heteroaryl comprises 1 heteroatom selected from N, or S, and wherein said phenyl and 5- to 6-membered heteroaryl are unsubstituted or substituted with 1, or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen, or C₁-C₄ alkyl.

In an embodiment of the invention, the compound of formula (II) may be a compound of formula (II-C), wherein B¹ and B² are CH; and wherein A is A2:

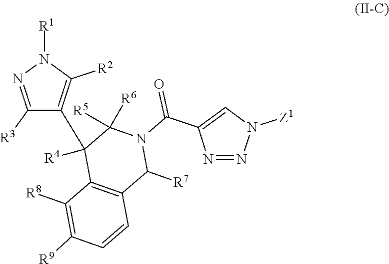

R¹, R², R³, R⁴, R⁵, R⁶, R⁷, R⁸, R⁹ and Z¹ are as defined for the compounds of formula (I) according to the present invention.

In a variant of this embodiment of the invention, the compound of formula (II-C) may be a compound of formula (II-C₁) wherein B¹ and B² are CH; R³, R⁵, R⁶ are hydrogen; and A is A2

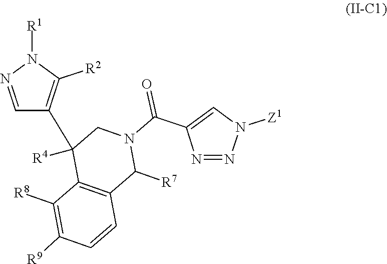

wherein R¹, R², R⁴, R⁷, R⁸, R⁹ and Z¹ are as defined for the compounds of formula (I) according to the present invention.

Preferably in the compound of formula (II-C₁) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy; and
Z¹ is defined as for the compounds of formula (I) according to the present invention.

Preferably in the compound of formula (II-C₁) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy; and
Z¹ is phenyl, a 5- to 6-membered heteroaryl, or C₃-C₆-cycloalkyl, wherein said 5- or 6-membered heteroaryl comprises 1 heteroatom selected from N, or S, and wherein said phenyl and 5- to 6-membered heteroaryl are unsubstituted or substituted with 1, or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen, or C₁-C₄ alkyl.

In an embodiment of the invention, the compound of formula (II) may be a compound of formula (II-D), wherein B¹ and B² are CH, and wherein A is A3:

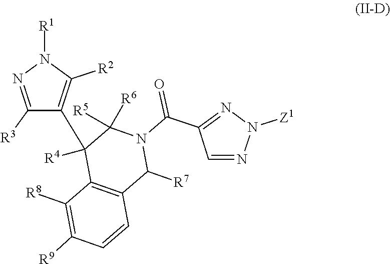

R¹, R², R³, R⁴, R⁵, R⁶, R⁷, R⁸, R⁹ and Z¹ are as defined for the compounds of formula (I) according to the present invention.

In a variant of this embodiment of the invention, the compound of formula (II-D) may be a compound of formula (II-D1) wherein B¹ and B² are CH; R³, R⁵, R⁶ are hydrogen; and A is A3

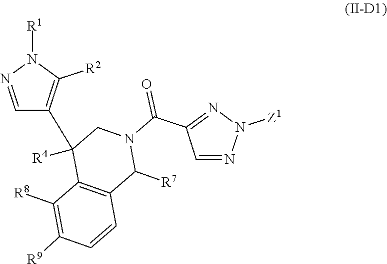

wherein R¹, R², R⁴, R⁷, R⁸, R⁹ and Z¹ are as defined for the compounds of formula (I) according to the present invention.

Preferably in the compound of formula (II-D1) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy; and
Z¹ is defined as for the compounds of formula (I) according to the present invention.

Preferably in the compound of formula (II-D1) of the invention
R¹ is C₁-C₄ alkyl, preferably methyl, ethyl, or isopropyl;
R² is hydrogen, fluorine, chlorine, or methyl;
R⁴ is of hydrogen, or methyl;
R⁷ is hydrogen, C₁-C₄ alkyl, or C₃-C₆-cycloalkyl;
R⁸ and R⁹ are independently selected from hydrogen, halogen, cyano C₁-C₄ alkyl, or C₁-C₄ alkoxy; and
Z¹ is phenyl, a 5- to 6-membered heteroaryl, or C₃-C₆-cycloalkyl, wherein said 5- or 6-membered heteroaryl comprises 1 heteroatom individually selected from N, or S, and wherein said phenyl and 5- to 6-membered heteroaryl are unsubstituted or substituted with 1, or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen, or C₁-C₄ alkyl.

The below intermediates are novel and as such form a further aspect of the invention.

According to a sixth aspect of the invention, there is provided an intermediate compound of formula (III), or a salt thereof:

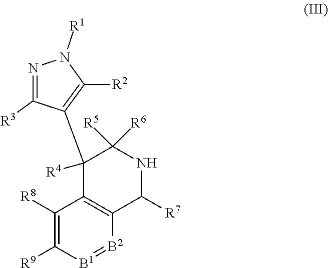

wherein R¹, R², R³, R⁴, R⁵, R⁶, R⁷, R⁸, R⁹, B¹ and B² are defined as for the compounds of formula (I) according to the present invention.

The intermediate compounds of formula (III) possess the same definitions for R¹, R², R³, R⁴, R⁵, R⁶, R⁷, R⁸, R⁹, B¹ and B² as for the compounds of formula (I) according to the invention and their corresponding preferences.

The presence of one or more possible asymmetric carbon atoms in a compound of formula (III) according to the invention means that the compounds may occur in chiral isomeric forms, i.e., enantiomeric or diastereomeric forms.

According to a seventh aspect of the invention, there is provided an intermediate compound of formula (IV):

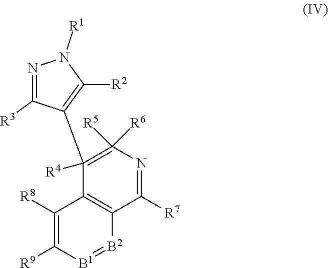

wherein R¹, R², R³, R⁴, R⁵, R⁶, R⁷, R⁸, R⁹, B¹ and B² are defined as for the compounds of formula (I) according to the present invention.

The intermediate compounds of formula (IV) possess the same definitions for R¹, R², R³, R⁴, R⁵, R⁶, R⁷, R⁸,

R⁹, B¹ and B² as for the compounds of formula (I) according to the invention and their corresponding preferences.

The presence of one or more possible asymmetric carbon atoms in a compound of formula (IV) according to the invention means that the compounds may occur in chiral isomeric forms, i.e., enantiomeric or diastereomeric forms.

According to an eighth aspect of the invention, there is provided an intermediate compound of formula (IIb) or a salt thereof:

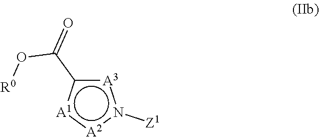

wherein R⁹ is C₁-C₆alkyl and wherein A1, A2, A3 and Z¹ are defined as for the compounds of formula (I) according to the present invention.

The intermediate compounds of formula (IIb) possess the same definitions for A1, A2, A3 and Z¹ as for the compounds of formula (I) according to the invention and their corresponding preferences.

In one embodiment R⁹ is C₁-C₄ alkyl, A1, A2, A3 are N and Z¹ is as defined for the compounds of formula (I) according to the present invention.

In one embodiment, the compound of formula (IIb) may be a compound of formula (IIb-1):

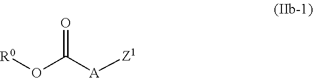

wherein
Z¹ is as defined for the compounds of formula (I) according to the present invention, R⁹ is as defined for compounds of formula (IIb) and A is selected from A1, A2, A3, A4, A5, A6, A7, or A8 as defined above for compounds of formula (II).

Preferably in the compound of formula (IIb-1) A is selected from

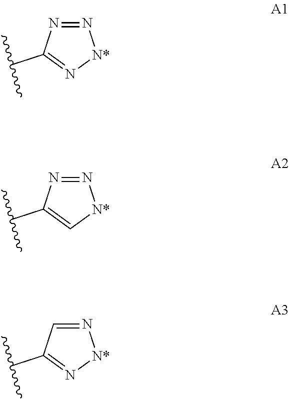

wherein

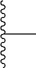

indicates the bond to the C(═O) group and the star (*) the Nitrogen with the bond to the Z¹ group, wherein Z¹ is as defined for the compounds of formula (I) according to the present invention and R⁹ is as defined for compounds of formula (IIb).

More preferably in the compound of formula (IIb-1) A is selected from A1 or A2, wherein Z¹ is as defined for the compounds of formula (I) according to the present invention, and R⁹ is as defined for compounds of formula (IIb).

In another embodiment of the invention, in the compound of formula (IIb-1) A is A1, wherein Z¹ is as defined for the compounds of formula (I) according to the present invention, and R⁹ is as defined for compounds of formula (IIb).

The presence of one or more possible asymmetric carbon atoms in any of the compounds selected from compounds of formula (I), (II), (II-A), (II-A1), (II-B), (II-B1), (II-C), (II-C₁), (II-D), or (II-D1), or compounds selected from compounds listed in Tables A-1 to A-24, or Table P (below), according to the invention means that the compounds may occur in chiral isomeric forms, i.e., enantiomeric or diastereomeric forms.

More preferably, the compound of formula (I) according to the invention is selected from compounds listed in any one of Tables A-1 to A-24.

Even more preferably the compound of formula (I) according to the invention is selected from compounds listed in Table P (below).

In one embodiment of the invention, the compound of formula (I) is selected from [1-(2-fluorophenyl)pyrazol-3-yl]-[4-(1-methylpyrazol-4-yl)-3,4-dihydro-1H-isoquinolin-2-yl]methanone, [1-(2,4-difluorophenyl)pyrazol-3-yl]-[4-(1-methylpyrazol-4-yl)-3,4-dihydro-1H-isoquinolin-2-yl]methanone, [1-(2-fluorophenyl)pyrazol-4-yl]-[4-(1-methylpyrazol-4-yl)-3,4-dihydro-1H-isoquinolin-2-yl]methanone, [2,5-dimethyl-1-(2,2,2-trifluoroethyl)pyrrol-3-yl]-[4-(1-methylpyrazol-4-yl)-3,4-dihydro-1H-isoquinolin-2-yl]methanone, (1,5-dimethylpyrazol-3-yl)-[4-(1-methylpyrazol-4-yl)-3,4-dihydro-1H-isoquinolin-2-yl]methanone, (2-phenyltriazol-4-yl)-[4-(1,3,5-trimethylpyrazol-4-yl)-3,4-dihydro-1H-isoquinolin-2-yl]methanone, (1-phenyltriazol-4-yl)-[4-(1,3,5-trimethylpyrazol-4-yl)-3,4-dihydro-1H-isoquinolin-2-yl]methanone, [1-(2,4-difluorophenyl)triazol-4-yl]-[4-(1-methylpyrazol-4-yl)-3,4-dihydro-1H-isoquinolin-2-yl]methanone, [1-(4-fluorophenyl)triazol-4-yl]-[4-(1-methylpyrazol-4-yl)-3,4-dihydro-1H-isoquinolin-2-yl]methanone, [4-(1-methylpyrazol-4-yl)-3,4-dihydro-1H-isoquinolin-2-yl]-(1-phenylimidazol-4-yl)methanone, [4-(1-methylpyrazol-4-yl)-3,4-dihydro-1H-isoquinolin-2-yl]-(1-phenyltriazol-4-yl)methanone, [2,5-dimethyl-1-(5-methylisoxazol-3-yl)pyrrol-3-yl]-[4-(1-methylpyrazol-4-yl)-3,4-dihydro-1H-isoquinolin-2-yl]methanone, (1-benzyltriazol-4-yl)-[4-(1-methylpyrazol-4-yl)-3,4-dihydro-1H-isoquinolin-2-yl]methanone, [4-(1-methylpyrazol-4-yl)-3,4-dihydro-1H-isoquinolin-2-yl]-(2-phenyltetrazol-5-yl)methanone, (1-isopropyltriazol-4-yl)-[4-(1-methylpyrazol-4-yl)-3,4-dihydro-1H-isoquinolin-2-yl]methanone, [4-(1-methylpyrazol-4-yl)-3,4-dihydro-1H-isoquinolin-2-yl]-(1-phenylpyrazol-3-yl)methanone, [1-(4-fluorophenyl)-5-methyl-triazol-4-yl]-[4-(1-methylpyrazol-4-yl)-3,4-dihydro-1H-isoquinolin-2-yl]methanone, [2-(4-fluorophenyl)triazol-4-yl]-[4-(1-methylpyrazol-4-yl)-3,4-dihydro-1H-isoquinolin-2-yl]methanone, [4-(1-methylpyrazol-4-yl)-3,4-dihydro-1H-isoquinolin-2-yl]-(1-phenyl-1,2,4-triazol-3-yl)methanone, (1-tert-butylimidazol-4-yl)-[4-(1-methylpyrazol-4-yl)-3,4-dihydro-1H-isoquinolin-2-yl]methanone, [1-(4-fluorophenyl)pyrazol-3-yl]-[4-(1-methylpyrazol-4-yl)-3,4-dihydro-1H-isoquinolin-2-yl]methanone, (5-cyclopropyl-1-methyl-pyrazol-3-yl)-[4-(1-methylpyrazol-4-yl)-3,4-dihydro-1H-isoquinolin-2-yl]methanone, [1-(3-fluoro-2-pyridyl)-1,2,4-triazol-3-yl]-[4-(1-methylpyrazol-4-yl)-3,4-dihydro-1H-isoquinolin-2-yl]methanone, [1-(3-fluorophenyl)-3-methyl-pyrazol-4-yl]-[4-(1-methylpyrazol-4-yl)-3,4-dihydro-1H-isoquinolin-2-yl]methanone, (5-benzyl-1-methyl-pyrazol-3-yl)-[4-(1-methylpyrazol-4-yl)-3,4-dihydro-1H-isoquinolin-2-yl]methanone, [4-(1-methylpyrazol-4-yl)-3,4-dihydro-1H-isoquinolin-2-yl]-[1-[5-(trifluoromethyl)-2-pyridyl]triazol-4-yl]methanone, [1-(2-methoxyphenyl) imidazol-4-yl]-[4-(1-methylpyrazol-4-yl)-3,4-dihydro-1H-isoquinolin-2-yl]methanone, (5-cyclopropyl-1-ethyl-pyrazol-3-yl)-[4-(1-methylpyrazol-4-yl)-3,4-dihydro-1H-isoquinolin-2-yl]methanone, [1-(2,6-difluorophenyl)pyrazol-3-yl]-[4-(1-methylpyrazol-4-yl)-3,4-dihydro-1H-isoquinolin-2-yl]methanone, [1-(3,4-difluorophenyl)pyrazol-3-yl]-[4-(1-methylpyrazol-4-yl)-3,4-dihydro-1H-isoquinolin-2-yl]methanone, [2-(2,4-difluorophenyl)tetrazol-5-yl]-[4-(1,5-dimethylpyrazol-4-yl)-3,4-dihydro-1H-isoquinolin-2-yl]methanone, [2-(2,4-difluorophenyl)tetrazol-5-yl]-[4-methyl-4-(1-methylpyrazol-4-yl)-1,3-dihydroisoquinolin-2-yl]methanone, [1-(2,4-difluorophenyl)-1,2,4-triazol-3-yl]-[4-(1,5-dimethylpyrazol-4-yl)-3,4-dihydro-1H-isoquinolin-2-yl]methanone, [2-(2,4-difluorophenyl)tetrazol-5-yl]-[6-methoxy-1-methyl-4-(1-methylpyrazol-4-yl)-3,4-dihydro-1H-isoquinolin-2-yl]methanone, [6-methoxy-1-methyl-4-(1-methylpyrazol-4-yl)-3,4-dihydro-1H-isoquinolin-2-yl]-(2-phenyltetrazol-5-yl)methanone, [(1R,4R)-6-methoxy-1-methyl-4-(1-methylpyrazol-4-yl)-3,4-dihydro-1H-isoquinolin-2-yl]-(2-phenyltetrazol-5-yl)methanone, [(1S,4S)-6-methoxy-1-methyl-4-(1-methylpyrazol-4-yl)-3,4-dihydro-1H-isoquinolin-2-yl]-(2-phenyltetrazol-5-yl)methanone, [2-(2,4-difluorophenyl)tetrazol-5-yl]-[(1S,4S)-6-methoxy-1-methyl-4-(1-methylpyrazol-4-yl)-3,4-dihydro-1H-isoquinolin-2-yl]methanone, [2-(2,4-difluorophenyl)tetrazol-5-yl]-[(1R,4R)-6-methoxy-1-methyl-4-(1-methylpyrazol-4-yl)-3,4-dihydro-1H-isoquinolin-2-yl]methanone, [2-(2,4-difluorophenyl)triazol-4-yl]-[rac-(1S,4S)-4-(1,5-dimethylpyrazol-4-yl)-1-methyl-3,4-dihydro-1H-isoquinolin-2-yl]methanone, [1-(2,4-difluorophenyl)triazol-4-yl]-[rac-(1S,4S)-4-(1,5-dimethylpyrazol-4-yl)-1-methyl-3,4-dihydro-1H-isoquinolin-2-yl]methanone, or [2-(2,4-difluorophenyl)tetrazol-5-yl]-[rac-(1S,4S)-4-(1,5-dimethylpyrazol-4-yl)-1-methyl-3,4-dihydro-1H-isoquinolin-2-yl]methanone.

In a preferred embodiment of the invention, the compound of formula (I) is selected from [(1S,4S)-4-(5-chloro-1-methyl-pyrazol-4-yl)-1-methyl-3,4-dihydro-1H-isoquinolin-2-yl]-[1-(2,4-difluorophenyl)triazol-4-yl]methanone, [(1S,4S)-4-(5-chloro-1-methyl-pyrazol-4-yl)-1-methyl-3,4-dihydro-1H-isoquinolin-2-yl]-[1-(3,5-difluoro-2-pyridyl)triazol-4-yl]methanone, [1-(3,5-difluoro-2-pyridyl)triazol-4-yl]-[(1S,4S)-4-(1,5-dimethylpyrazol-4-yl)-1-methyl-3,4-dihydro-1H-isoquinolin-2-yl]methanone, [2-(2,4-difluorophenyl)tetrazol-5-yl]-[(1S,4S)-4-(1,5-dimethylpyrazol-4-yl)-1-methyl-3,4-dihydro-1H-isoquinolin-2-yl]methanone, [1-(3,5-difluoro-2-pyridyl)triazol-4-yl]-[rac-(1S,4S)-4-(5-chloro-1-methyl-pyrazol-4-yl)-1-methyl-3,4-dihydro-1H-isoquinolin-2-yl]methanone, [1-(3,5-difluoro-2-pyridyl)triazol-4-yl]-[(1S,4S)-1-methyl-4-(1-methylpyrazol-4-yl)-3,4-dihydro-1H-isoquinolin-2-yl]methanone, [(1S,4S)-4-(5-chloro-1-methyl-pyrazol-4-yl)-1-methyl-3,4-dihydro-1H-isoquinolin-2-yl]-[2-(2,4-difluorophenyl)tetrazol-5-yl]methanone, [1-(2,4-difluorophenyl)triazol-4-yl]-[(1S,4S)-4-(1,5-dimethylpyrazol-4-yl)-1-methyl-3,4-dihydro-1H-isoquinolin-2-yl]methanone, [6-methoxy-1-methyl-4-(1-methylpyrazol-4-yl)-3,4-dihydro-1H-isoquinolin-2-yl]-(2-phenyltetrazol-5-yl)methanone, [2-(2,4-difluorophenyl)tetrazol-5-yl]-[4-(1,5-dimethylpyrazol-4-yl)-3,4-dihydro-1H-isoquinolin-2-yl]methanone [4-(1-methylpyrazol-4-yl)-3,4-dihydro-1H-isoquinolin-2-yl]-(2-phenyltetrazol-5-yl)methanone, [(1S,4S)-6-methoxy-1-methyl-4-(1-methylpyrazol-4-yl)-3,4-dihydro-1H-isoquinolin-2-yl]-(2-phenyltetrazol-5-yl)methanone, [1-(2,4-difluorophenyl)triazol-4-yl]-[4-(1,5-dimethylpyrazol-4-yl)-3,4-dihydro-1H-isoquinolin-2-yl]methanone, [2-(2,4-difluorophenyl)tetrazol-5-yl]-[6-methoxy-1-methyl-4-(1-methylpyrazol-4-yl)-3,4-dihydro-1H-isoquinolin-2-yl]methanone, [2-(2,4-difluorophenyl)tetrazol-5-yl]-[(1S,4S)-6-methoxy-1-methyl-4-(1-methylpyrazol-4-yl)-3,4-dihydro-1H-isoquinolin-2-yl]methanone, [(1R,4R)-6-methoxy-1-methyl-4-(1-methylpyrazol-4-yl)-3,4-dihydro-1H-isoquinolin-2-yl]-(2-phenyltetrazol-5-yl)methanone, [1-(3,5-difluoro-2-pyridyl)triazol-4-yl]-[(1R,4R)-4-(1,5-dimethylpyrazol-4-yl)-1-methyl-3,4-dihydro-1H-isoquinolin-2-yl]methanone, [(1R,4R)-4-(5-chloro-1-methyl-pyrazol-4-yl)-1-methyl-3,4-dihydro-1H-isoquinolin-2-yl]-[1-(2,4-difluorophenyl)triazol-4-yl]methanone, (1-phenyltriazol-4-yl)-[4-(1,3,5-trimethylpyrazol-4-yl)-3,4-dihydro-1H-isoquinolin-2-yl]methanone, [4-(1-methylpyrazol-4-yl)-3,4-dihydro-1H-isoquinolin-2-yl]-(1-phenyltriazol-4-yl)methanone, [2-(4-fluorophenyl)triazol-4-yl]-[4-(1-methylpyrazol-4-yl)-3,4-dihydro-1H-isoquinolin-2-yl]methanone, [2-(2,4-difluorophenyl)triazol-4-yl]-[(1S,4S)-4-(1,5-dimethylpyrazol-4-yl)-1-methyl-3,4-dihydro-1H-isoquinolin-2-yl]methanone, [(1S,4S)-4-(5-chloro-1-methyl-pyrazol-4-yl)-1-methyl-3,4-dihydro-1H-isoquinolin-2-yl]-[2-(2,4-difluorophenyl)triazol-4-yl]methanone, or [2-(2,4-difluorophenyl)tetrazol-5-yl]-[4-methyl-4-(1-methylpyrazol-4-yl)-1,3-dihydroisoquinolin-2-yl]methanone.

The compounds of formula (I) according to the present invention can be made as shown in the following Schemes below, in which, unless otherwise stated, the definition of each variable is as defined above for a compound of formula (I).

The compounds of formula (I) according to the present invention can be made as shown in the following Schemes 1 to 19, in which, unless otherwise stated, the definition of each variable is as defined above for a compound of formula (I).

In particular, compounds of formula (I), wherein R⁴ and R⁶ are hydrogen and R⁵ is hydrogen or methyl, can be made as shown in the following Schemes, in which, unless otherwise stated, the definition of each variable is as defined above for a compound of formula (I).

In any of the Schemes 1 to 19 below, the presence of one or more possible asymmetric carbon atoms in a compound of formula (I) according to the invention means that the compounds may occur in chiral isomeric forms, i.e., enantiomeric or diastereomeric forms.

Compounds of formula (I) may be prepared by a person skilled in the art following known methods. More specifically, compounds of formula (I) may be prepared from compounds of formula (III), or a salt thereof, wherein R¹, R², R³, R⁴, R⁵, R⁶, R⁷, R⁸, R⁹, B¹ and B² are as defined above for the compound of formula (I) by reaction with a compound of formula (II), wherein A1, A2, A3 and Z¹ are as defined above for the compound of formula (I). This reaction is shown in Scheme 1.

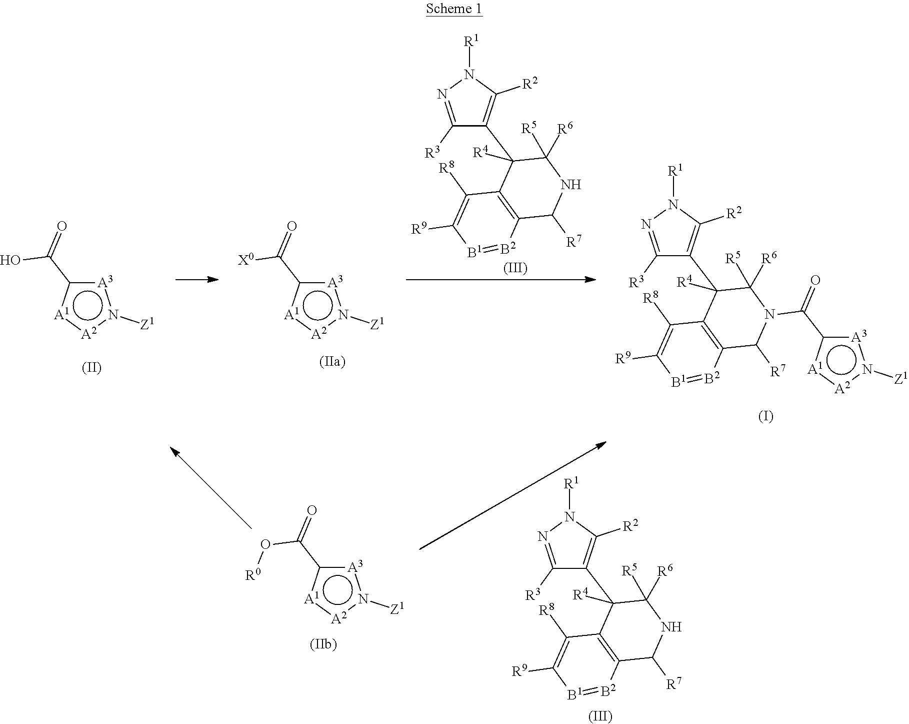

In Scheme 1, compounds of formula (II), wherein A1, A2, A3 and Z¹ are as defined above for the compound of formula (I), are activated to compounds of formula (IIa) by methods known to a person skilled in the art and described, for example, in Tetrahedron 2005, 61 (46), 10827-10852. For example, compounds of formula (IIa), where X⁰ is halogen, are formed by treatment of compounds of formula (II) with, for example, oxalyl chloride or thionyl chloride in the presence of catalytic quantities of N, N-dimethylformamide (DMF) in inert solvents such as methylene dichloride or tetrahydrofuran (THF) at temperatures between 20° C. to 100° C., preferably 25° C. Treatment of compounds of formula (IIa) with compounds of formula (III), wherein R¹, R², R³, R⁴, R⁵, R⁶, R⁷, R⁸, R⁹, B¹ and B² are as defined above for the compound of formula (I), optionally in the presence of a base, e.g. triethylamine or pyridine, leads to compounds of formula (I). Alternatively, compounds of formula (I) may be prepared by treatment of compounds of formula (II) with dicyclohexyl carbodiimide (DCC), 1-ethyl-3-(3-dimethylaminopropyl) carbodiimide (EDC) or 1-[bis(dimethylamino)methylene]-1H-1,2,3-triazolo[4,5-b]pyridinium 3-oxide hexafluorophosphate (HATU) to give the activated compound of formula (IIa), wherein X⁰ is G⁰¹, G⁰², or G⁰³ as set forth below, in an inert solvent, e.g. pyridine, DMF, acetonitrile, CH₂Cl₂, or THF, optionally in the presence of a base, e.g. triethylamine, at temperatures between 30° C. and 180° C. Finally, a compound of formula (II) can also be activated by reaction with a coupling reagent such as propanephosphonic acid anhydride (T3P) to provide compounds of formula (IIa), wherein X⁰ is G⁰⁴ as set forth below, as described for example in Synthesis 2013, 45, 1569. Further reaction with an amine (or a salt thereof) of the compound of formula (III) leads to compounds of formula (I).

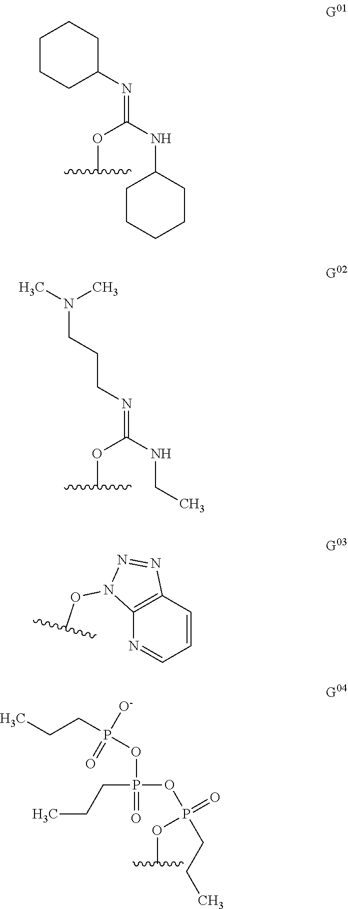

Compounds of formula (II) can be prepared from compounds of formula (IIb), wherein A1, A2, A3 are N and Z¹ are as described in formula (I), and R⁹ is C₁-C₄alkyl, by ester hydrolysis. A variety of conditions can be used, as for example aqueous sodium hydroxide, or lithium hydroxide and an organic water miscible solvent like THF, or dimethoxyethane, or methanol, or ethanol. Such ester hydrolyses are well known to those skilled in the art. Compounds of formula (IIb) can also be directly converted to compounds of formula (I) by reacting compounds of formula (IIb) with compounds of formula (III) in the presence of trimethyl aluminium, or trimethyl aluminium-DABCO complex in an inert solvent such as toluene or methylene chloride. Such reactions have been reported in the literature (see Tetrahedron Lett. 1977, 4171-4174, and Tetrahedron Lett. 2006, 5767-5769, and references cited therein). Compounds of formula (II) and (IIb) are commercially available or can be synthesized as described vide infra.

Compounds of formula (Illa), wherein R⁴ and R⁶ are hydrogen, R⁵ is hydrogen, or methyl and R¹, R², R³, R⁷, R⁸, R⁹, B¹ and B² are as defined above for the compound of formula (I), may be prepared by a person skilled in the art following known methods.

For example, compounds of formula (Illa), wherein R⁴ and R⁶ are hydrogen, R⁵ is hydrogen, or methyl and R¹, R², R³, R⁷, R⁸, R⁹, B¹ and B² are as defined above for the compound of formula (I), may be prepared from compounds of formula (IVa), wherein R⁴ and R⁶ are hydrogen, R⁵ is hydrogen, or methyl and R¹, R², R³, R⁷, R⁸, R⁹, B¹ and B² are as defined above for the compound of formula (I), by treatment with a reducing agent such as NaBH₃CN and an acid, for example hydrochloric acid, or acetic acid in a protic solvent such as methanol or ethanol and the like. Such reactions are well known in the literature and analogous reactions have been described for example in Deng, Zeping et al, CN103772278, and Synthesis 1979, 4, 281-3. Alternatively, compounds of formula (Illa) may be prepared from compounds of formula (IVa) by reduction with hydrogen in the presence of a suitable metal catalyst, such as Pd, Ir, Rh with a suitable ligand, e.g., diphosphine [1,2-bis(diphenylphosphino) ethane (dppe), 1,3-bis(diphenylphosphino) propane (dppp), or 1,4-bis(diphenylphosphino) butane (dppb)]. Similar reactions have been reported for example in Reaction Kinetics and Catalysis Letters 2007, 92, 99-104. This reaction is shown in Scheme 2.

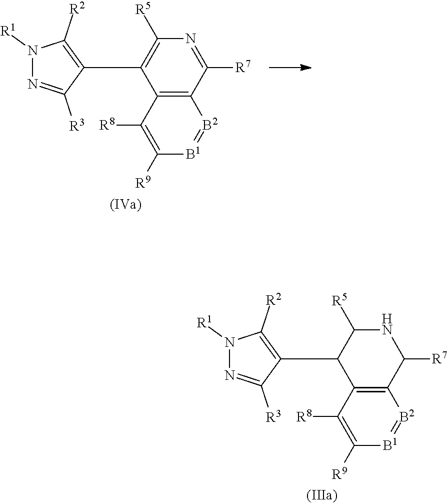

Alternatively, compounds of formula (Illa) may be prepared as shown in Scheme 3.

As shown in Scheme 3, compounds of formula (IIIb), wherein R⁴, R⁶ and R⁷ are hydrogen, R⁵ is hydrogen, or methyl and R¹, R², R³, R⁸, R⁹, B¹ and B² are as defined above for the compound of formula (I), can be converted to compounds of formula (V), wherein R⁴, R⁶ and R⁷ are hydrogen, R⁵ is hydrogen, or methyl and R¹, R², R³, R⁸, R⁹, B¹ and B² are as defined above for the compound of formula (I), by treatment of compounds of formula (IIIb) with a compound of formula (VI), wherein X⁰ is a leaving group, such as halogen, and R⁹ is C₁-C₆alkyl, by methods known to a person skilled in the art and by those described in Scheme 1. Alternatively, compounds of formula (V) may be prepared by treatment with an anhydride of formula (R⁰CO)₂O, wherein R⁹ is C₁-C₆alkyl, in an inert solvent such as methylene chloride, THF, or 2-methyl-THF, optionally in the presence of a base, such as triethylamine, or dimethylaminopyridine at temperatures between 0° C. and 60° C. Compounds of formula (V) are then metalated with a base, for example an alkyl metal base, such as tert-butyl lithium, and an additive such as N,N,N′,N′-tetramethylethylendiamine (TMEDA) at low temperature, for example −78° C. to room temperature, in an inert polar solvent such as THF, or 2-methyl-THF. Subsequent treatment of the anion of formula (V) formed under such conditions with an electrophile of formula R^{X}—X⁰, wherein X⁰ is as previously defined and R^{X} is C₁-C₄ alkyl, C₁-C₄ alkylcarbonyl, C₁-C₄ alkoxycarbonyl, N-methoxy-N-methyl-carbonyl, C₁-C₄ alkylaminocarbonyl, di(C₁-C₄ alkylamino) carbonyl or C₃-C₆ cycloalkyl, wherein said C₃-C₆-cycloalkyl is unsubstituted or substituted by 1, 2, or 3 substituents independently selected from halogen, cyano, C₁-C₄ alkyl, C₁-C₄ haloalkyl and C₁-C₄ alkoxy, yields compounds of formula (Va), wherein R⁴ and R⁶ are hydrogen, R⁵ is hydrogen, or methyl, R⁹ is C₁-C₆ alkyl and R¹, R², R³, R⁷, R⁸, R⁹, B¹ and B² are as defined above for the compound of formula (I). This reaction is shown in Scheme 3.

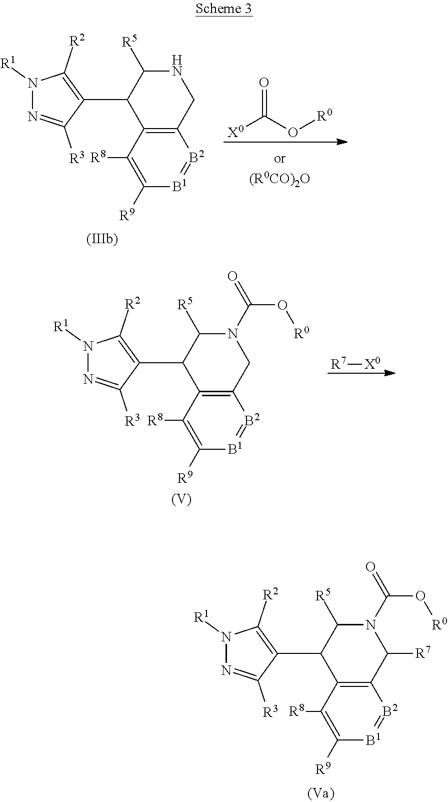

Compounds of formula (Va) may be converted to compounds of formula (Illa), wherein R⁴ and R⁶ are hydrogen, R⁵ is hydrogen, or methyl and R¹, R², R³, R⁷, R⁸, R⁹, B¹ and B² are as defined above for the compound of formula (I), by methods known to a person skilled in the art. For example, compounds of formula (Va), wherein R⁹ is tert-butyl, may be treated with an organic or inorganic acid such as trifluoroacetic acid or HCl to give compounds of formula (Illa). This reaction is shown in Scheme 4.

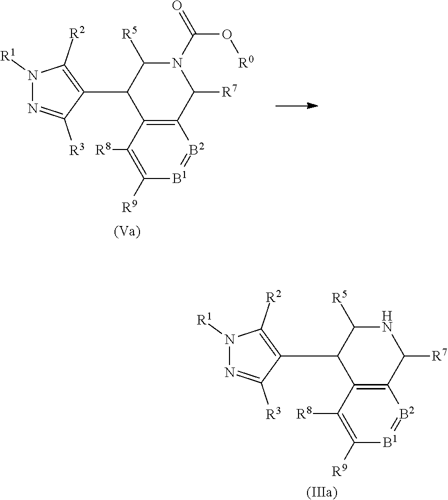

Compounds of formula (IVa), wherein R⁴ and R⁶ are hydrogen, R⁵ is hydrogen or methyl and R¹, R², R³, R⁷, R⁸, R⁹, B¹ and B² are as defined above for the compound of formula (I), may be prepared by reacting compounds of formula (VIII), wherein R¹, R² and R³ are as defined above for the compound of formula (I) and X⁰ is halogen, preferably chlorine, bromine or iodine, with compounds of formula (VII), wherein R⁵ is hydrogen or methyl and R⁷, R⁸, R⁹, B¹ and B² are as defined above for the compound of formula (I), by means of a C-C bond formation reaction typically under palladium-catalyzed (alternatively nickel-catalyzed) cross-coupling conditions. This reaction is shown in Scheme 5.

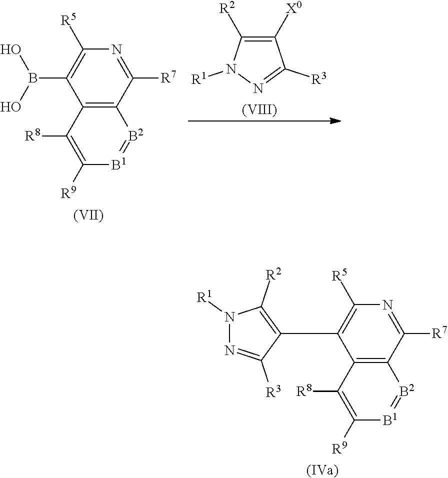

Suzuki-Miyaura cross-coupling reactions between compounds of formula (VIII) and compound of formula (VII) are well known to a person skilled in the art and are usually carried out in the presence of a palladium catalyst, such as tetrakis(triphenylphosphine)-palladium (0) or [1,1′-bis(diphenylphosphino) ferrocene]palladium (II) dichloride dichloromethane complex, and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such reactions have been reviewed for example in J. Organomet. Chem. 1999, 576, 147-168. A person skilled in the art will also recognize that the reaction can be reversed, i.e. by reacting a compound of formula (X), wherein R¹, R² and R³ are as defined above for the compound of formula (I), with a compound of formula (IX), wherein R⁵ is hydrogen or methyl, R⁷, R⁸, R⁹, B¹ and B² are as defined above for the compound of formula (I) and X⁰ is halogen, preferably chlorine, bromine or iodine, to provide a compound of formula (IVa), wherein R⁴ and R⁶ are hydrogen, R⁵ is hydrogen or methyl and R¹, R², R³, R⁷, R⁸, R⁹, B¹ and B² are as defined above for the compound of formula (I). This reaction is shown in Scheme 6.

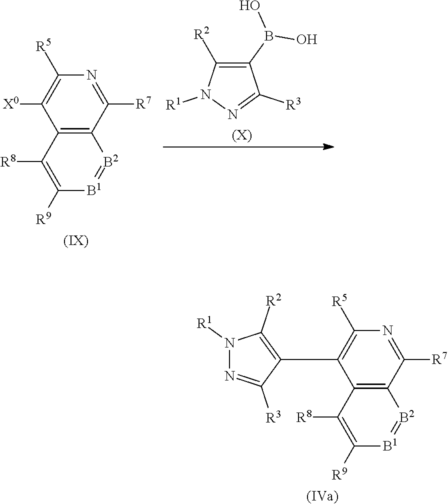

A further cross-coupling chemistry, namely C—H activation, can also be used to prepare compounds of formula (IVa), wherein R⁴ and R⁶ are hydrogen, R⁵ is hydrogen or methyl and R¹, R², R³, R⁷, R⁸, R⁹, B¹ and B² are as defined above for the compound of formula (I) (Scheme 7).

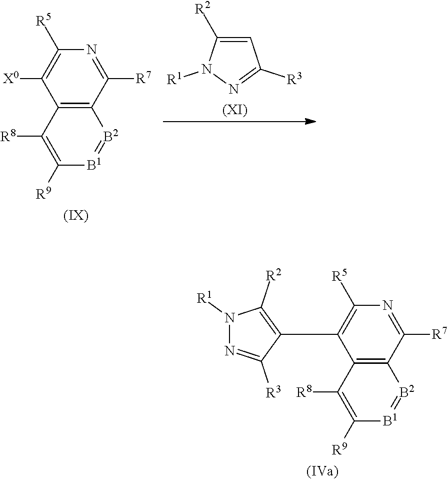

As shown in Scheme 7, compounds of formula (IX), wherein R⁵ is hydrogen or methyl, R⁷, R⁸, R⁹, B¹ and B² are as defined above for the compound of formula (I) and X⁰ is halogen, preferably chlorine, bromine or iodine, are reacted with compounds of formula (XI), wherein R¹, R² and R³ are as defined above for the compound of formula (I), in the presence of a palladium catalyst, typically palladium acetate Pd(OAc)₂, a suitable ligand, for example 1,10-phenanthroline, in the presence of a base such as cesium carbonate or potassium carbonate, in inert solvents such as chlorobenzene, toluene or xylene at temperatures between room temperature and 180° C., optionally under microwave heating conditions, preferably under inert atmosphere. Similar reactions have been reported in the literature for example in Chemical Science 2013, 4, 2374-2379.

Also, compounds of formula (III) may be prepared from compounds of formula (XVI) (Scheme 8).

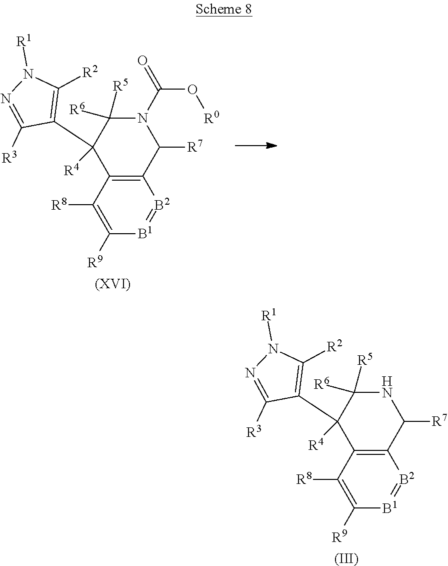

As shown in Scheme 8, compounds of formula (III) may be prepared by a person skilled in the art by a carbamate deprotection reaction of compounds of formula (XVI), wherein R¹, R², R³, R⁴, R⁵, R⁶, R⁷, R⁸, R⁹, B¹ and B² are as defined above for the compound of formula (I) and R⁰¹ may be a member of a common carbamate protecting group substituent, for example methyl, tert-butyl, allyl, 2,2,2-trichloroethyl or benzyl. For example, when R⁰¹ is methyl, a suitable solvent such as dichloromethane and a suitable reagent such as iodotrimethylsilane may be employed to afford the product upon heating at temperatures between room temperature and 200° C., preferably between 20° C. and the boiling point of the reaction mixture as described, for example, in J. Am. Chem. Soc. 1992, 114, 5959. The compounds of formula (III) thus obtained are converted to compounds of formula (I) (Scheme 1).

Compounds of formula (XVI), wherein R¹, R², R³, R⁴, R⁵, R⁶, R⁷, R⁸, R⁹, B¹ and B² are as defined above for the compound of formula (I) and wherein R⁰¹ is as described above, may be formed by a Pictet-Spengler reaction between an aldehyde (including formaldehyde in its various forms) of formula (XV), wherein R⁷ is as defined above for the compound of formula (I), and a compound of formula (XIV), wherein R¹, R², R³, R⁴, R⁵, R⁶, R⁸, R⁹, B¹ and B² are as defined above for the compound of formula (I) and wherein R⁰¹ is as described above, by combination with an acid in a suitable solvent, for example as described in Tetrahedron 1987, 43, 439 (Scheme 9).

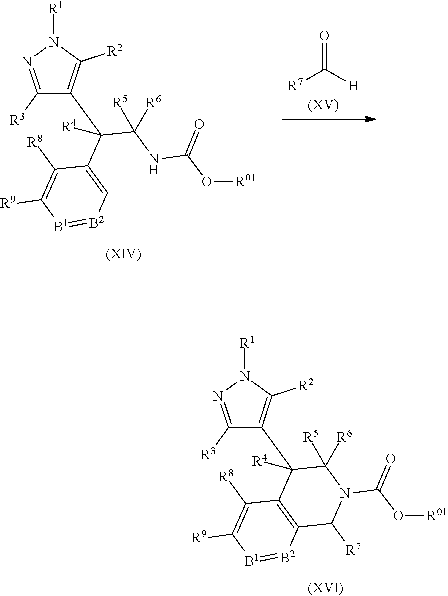

This Pictet-Spengler reaction is stereospecific when compounds of formula (XIV) wherein R¹, R², R³, R⁸, R⁹, are as described in formula (I), B¹ is CR¹⁰, B² is CR¹¹, and wherein R⁰¹ is as described above, namely compounds of formula (XIVa) are reacted with compounds of formula (XVa), wherein R⁷ is C₁-C₄alkyl to give racemic compounds of formula rac-syn-(XVIa) with a syn-relationship of the pyrazole ring and the R⁷ substituent (Scheme 10).

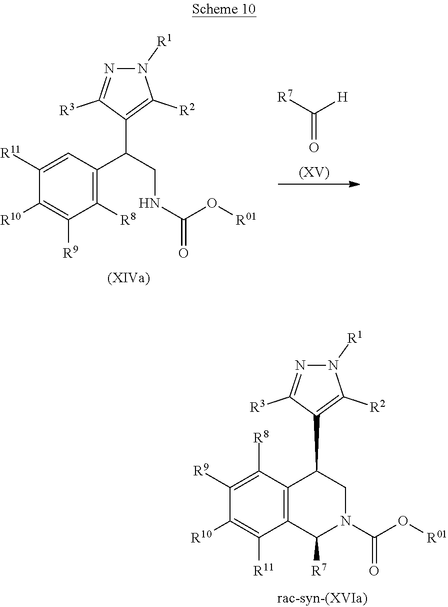

A further consequence of the stereospecific formation of compounds of formula rac-syn-(XVIa) in the Pictet-Spengler reaction is that if the synthesis begins with opticall pure compounds of formula (XIVa) then chiral compounds of formula (XVIa) can be obtained. This is illustrated in scheme 11 for the case of compounds of formula (XIV), wherein R¹, R², are methyl, R⁸, R⁹, R¹¹, and R¹¹ are hydrogen, and R⁹ is methyl, namely(S)-(XIVb), and (R)-(XIVb), which after Pictet-Spengler reaction with (XV) in which R⁷ is methyl, give (S,S)-syn-(XVIb) and (R,R)-syn-(XVIb), respectively

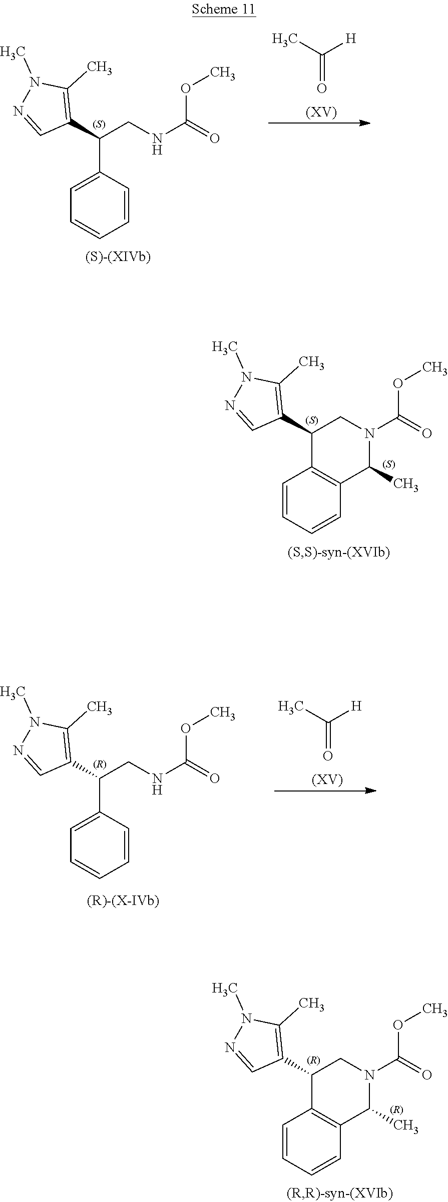

Compounds of formula (XIV), wherein R¹, R², R³, R⁴, R⁵, R⁶, R⁸, R⁹, B¹ and B² are as defined above for the compound of formula (I) and wherein R⁰¹ is as described above, may be prepared by a reaction between amines of formula (XIII), wherein R¹, R², R³, R⁴, R⁵, R⁶, R⁸, R⁹, B¹ and B² are as defined above for the compound of formula (I), and a suitable protecting reagent such as methyl chloroformate, optionally in the presence of a base such as triethylamine or pyridine, in a suitable solvent such as dichloromethane at temperatures between −20° C. and the boiling point of the mixture, as for example described in Org. Biomol. Chem. 2016 14, 6853 (Scheme 12).

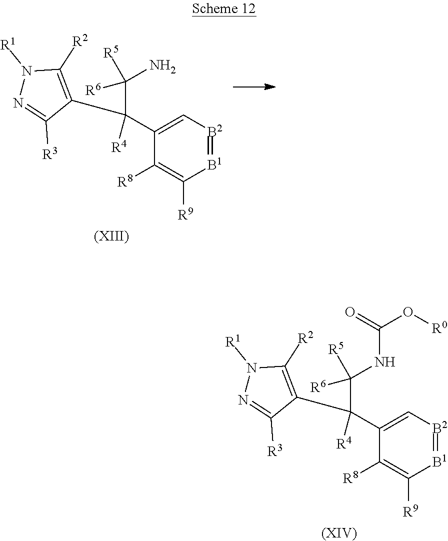

Compounds of formula (XIII), or salts thereof, wherein R¹, R², R³, R⁴, R⁵, R⁶, R⁸, R⁹, B¹ and B² are as defined above for the compound of formula (I), may be prepared by a person skilled in the art by a reaction between nitriles of formula (XII), wherein R¹, R², R³, R⁴, R⁸, R⁹, B¹ and B² are as defined above for the compound of formula (I), and a suitable nucleophile such as (dimethyl sulfide)dihydroboron (BMS) in a suitable aprotic solvent such as tetrahydrofuran, for example as described in J. Org. Chem. 1981, 47, 3153. The nitriles of formula (XII) can also be reduced to the primary amines with hydrogen in the presence of a catalyst, such as a Platinum salt, or Raney-Nickel in the presence of a hydrogen donor, for example ethanol or methanol. Many further conditions for reducing nitriles to primary amines which are well known to those skilled in the art. Alternatively, Grignard reagents R⁵MgBr or R⁶MgBr, wherein R⁵ and R⁶ are as defined above for the compound of formula (I), may be added as nucleophiles to compounds of formula (XII), sequentially or simultaneously, to allow more highly substituted amines of formula (XIII) to be prepared. Such Grignard additions to nitriles are carried out in an inert solvent such as diethyl ether, tert-butylmethyl ether, and cyclopentyl methyl ether in the presence of a Lewis acid such as Ti(O-ⁱPr)₄ (see Synlett (2007), (4), 652-654). This reaction is shown in Scheme 13.

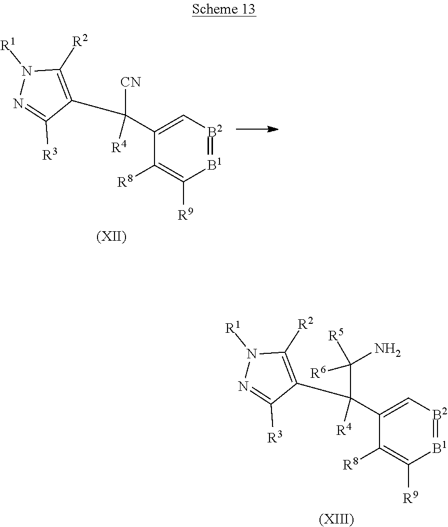

Compounds of formula (XII), wherein R¹, R², R³, R⁴, R⁸, R⁹, B¹ and B² are as defined above for the compound of formula (I), may be prepared by a person skilled in the art following known methods. More specifically, compounds of formula (XII), and intermediates thereof, may be prepared from compounds of formula (XVII) as shown in Scheme 14.

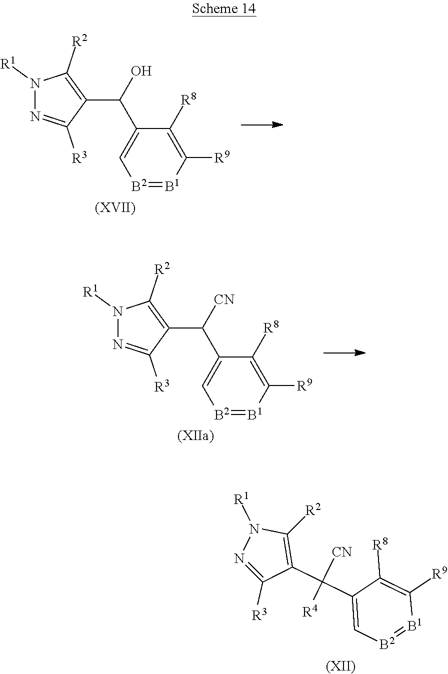

For example, compounds of formula (XII), wherein R¹, R², R³, R⁸, R⁹, B¹ and B² are as defined above for the compound of formula (I) and R⁴ is different from hydrogen, may be prepared by a person skilled in the art by deprotonation of compound of formula (XIIa) wherein R¹, R², R³, R⁸, R⁹, B¹ and B² are as defined above for the compound of formula (I), using a strong base such as n-butyl lithium or sodium hydride at cryogenic temperatures in an inert solvent such as tetrahydrofuran, followed by addition of a suitable alkylating agent R⁴—X, wherein R⁴ is C₁-C₄alkyl and X is halogen, for example iodomethane.

Compounds of formula (XIIa), wherein R⁴ is hydrogen and R¹, R², R³, R⁸, R⁹, B¹ and B² are as defined above for the compound of formula (I), may be prepared from alcohols of formula (XVII) by treatment with cyanotrimethylsilane (TMSCN) in the presence of a base such as lithium carbonate in a nonpolar solvent such as dichloromethane at temperatures between 0° C. and the boiling point of the reaction mixture. Such transformations are well known in the literature under a variety of conditions, for example as described in Org. Lett. 2008. 10, 4570 and references therein. This reaction is shown in Scheme 14.

A further synthesis of compounds of formula (III) wherein B¹ is CR¹⁰, B² is CR¹¹, R^{1a} is C₁-C₄alkyl, R^{2a} is hydrogen, halogen or C₁-C₄alkyl, R³ is hydrogen, R^{4a}, R^{5a}, R^{6a}R^{7a} are hydrogen or C₁-C₄ alkyl, and R⁸, R⁹, R¹⁰, and R¹¹ are as previously defined under formula (I), namely compounds of formula (IIIc);

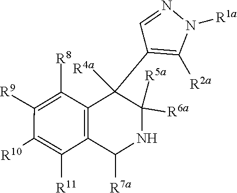

May also be prepared by treating compounds of formula (XVIII)

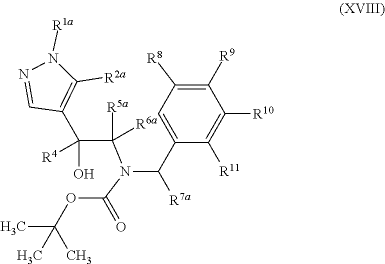

wherein R^{1a} is C₁-C₄alkyl, R^{2a} is hydrogen, halogen or C₁-C₄alkyl, R^{4a}, R^{5a}, R^{6a}, R^{7a} are hydrogen or C₁-C₄alkyl, and R⁸, R⁹, R¹⁰, and R¹¹ are as previously defined under formula (I), with strong acids, for example sulphuric, hydrochloric, hydrobromic, trifluoroacetic, trifllic, or methane sulphonic acids and the like, or Lewis acids, such as aluminium trichloride, or bismuth (III) triflate, in an inert solvent such as chlorobenzene, nitrobenzene at temperature between 0° C. to 180° C. to yield compounds of formula (IIIc). These are converted to compounds of formula (I) as previously described vide supra. Those skilled in the art will realize that such cyclisation's can proceed through intermediates such as compounds of formula (XIX),

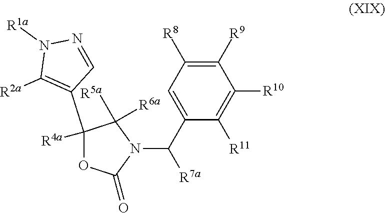

and (when R^{4a} is methyl), compound of formula (XX)

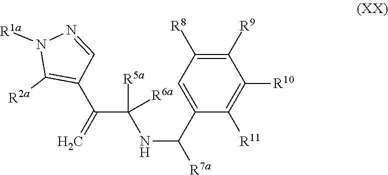

and wherein the substituents R^{1a}, R^{2a}, R^{3a}, R^{4a}, R^{5a}, R^{6a}, R^{7a}, R⁸, R⁹, R¹⁰, and R¹¹ in these compounds are as previously described. Depending on the conditions of the reaction these intermediates can be isolated and/or converted further directly to compounds of formula (IIIc). Those skilled in the art will also realize that when R⁷ is C₁-C₄alkyl, mixtures of diastereoisomers racemic-(syn-IIIc) and racemic-(anti-IIIc) can be obtained in ratios that can be controlled to direct preferential formation of one isomer over the other (scheme 15).

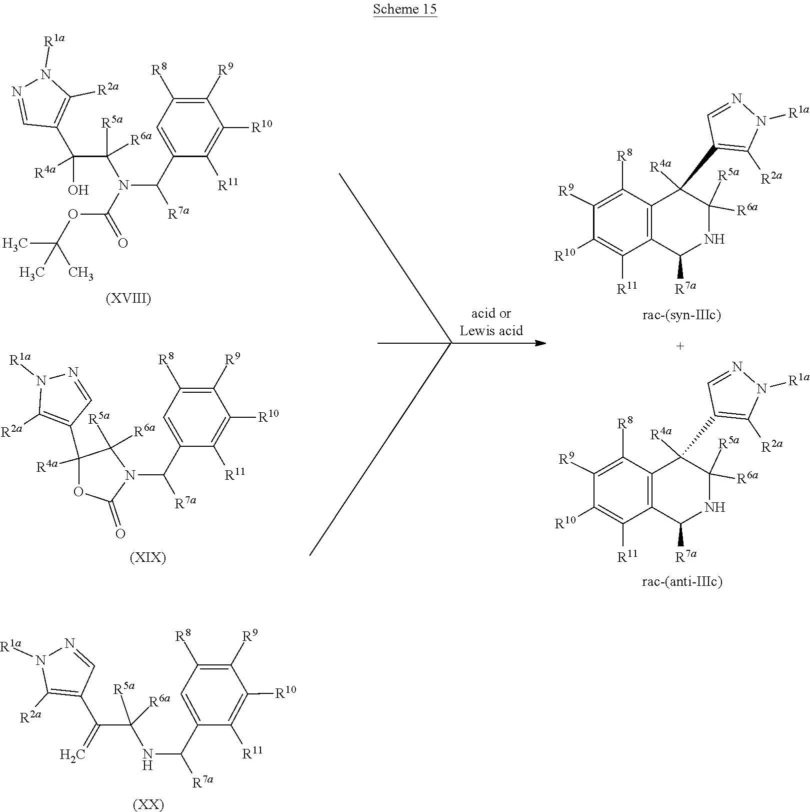

Compounds (XVIII), (XIX), and (XX) can be prepared as shown in the following scheme 16, and as described in the experimental section.

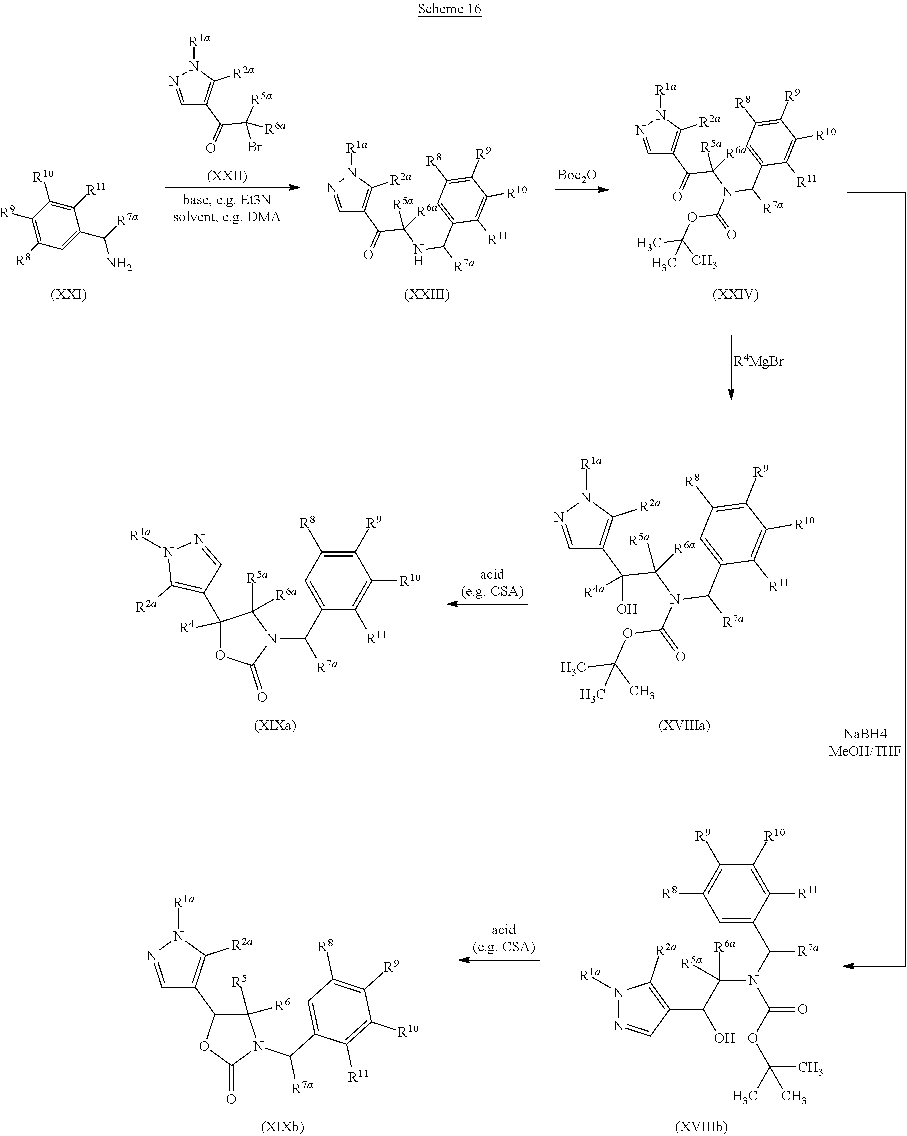

As shown in scheme 16, a benzyl amine of formula (XXI) is used to alkylate a compound of formula (XXII), in the presence of a base, such as triethylamine (Et₃N) in an inert solvent, such as DMF or DMA. The compound 5 (XXIII) so obtained, may be isolated, or treated directly with BOC-anhydride in situ, to give a compound of formula (XXIV). Compound of formula XXIV can be reduced with a hydride source (e.g. NaBH₄ in MeOH/THF) to give the target molecule (XVIIIb), which can then be cyclized with, for example with camphor sulphonic acid in EtOAc, to give compounds of formula (XIXb). Alternatively, compounds of formula (XXIV) can be reacted with a Grignard reagent R⁴MgBr in an inert ethereal solvent (e.g. THF) to give compounds of formula (XVIIIa), which can the cyclized with camphorsulphonic acid in, e.g. EtOAc, to give compounds of formula (XIXa). In 10 compounds (XIX) and (XVIII) R^{1a} is C₁-C₄alkyl, R^{2a} is hydrogen, halogen, or C₁-C₄alkyl, R^{4a} is hydrogen or C₁-C₄alkyl, R^{5a}, R^{6a}, and R^{7a} are hydrogen or C₁-C₄ alkyl, and R⁸, R⁹, R¹⁰, and R¹¹ are as previously defined under formula (I).

A further aspect of this Friedel-Crafts chemistry should be noted. If the chemistry is carried out starting with a chiral amine (XXI), i.e. R^{7a} is C₁-C₄alkyl, the stereochemistry is retained in the final compounds of formula (I). This is illustrated below in scheme 17, for when R^{7a} is methyl:

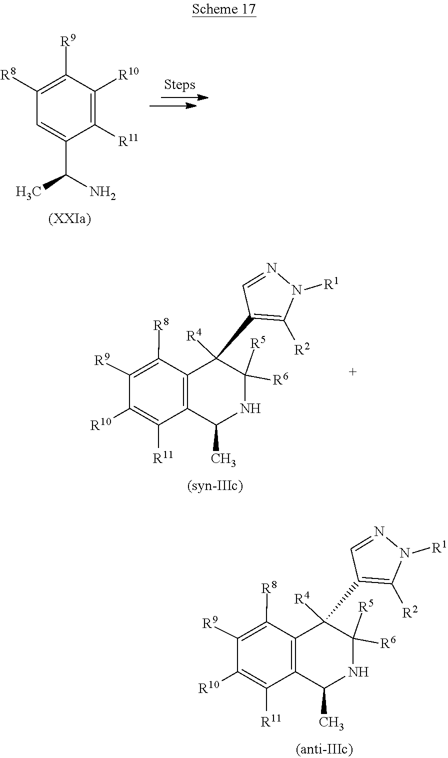

Compounds of formula (XVII) may be prepared by methods known to a person skilled in the art. Compounds of formula (XXI) and (XXII) are easily prepared by those skilled in the art or can be purchased.

Compounds of formula II are either commercially available or can be synthesized as described vide infra.

Compounds of formula (IIb) wherein A1, A2, A3 are N and Z¹ are as described for compounds of formula (I) of the present invention, and wherein R⁹ is C₁-C₆alkyl, namely compounds of formula (IIba)

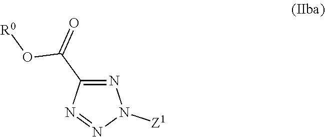

can be obtained by reacting a diazonium salt of formula (XXV)

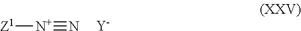

wherein Z¹ is as defined for compounds of formula (I) of the present invention and Y-being the counterion depending on the conditions in which the diazotation step is performed, for example Cl⁻ or BF₄⁻, and compounds of formula (XXVI),

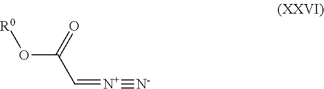

wherein R⁰ is C₁-C₆alkyl. This reaction can be catalyzed with various silver salts, a preferred one being silver acetate, and performed in a variety of solvents, for example THF, DMF or toluene or a mixture thereof, usually at temperatures between 0° C. and 25° C., in presence of at least one equivalent of a base, for example sodium carbonate. These dipolar [3+2] cycloadditions are highly regioselective and are described, for example in Tetrahedron 2020, 76 (14), 131063. Compounds of formula (XXV) as defined just before, can be prepared from primary amines of formula (XXVII) by reaction with a diazotation reagent, for example a salt of nitrous acid, for example sodium nitrite. The solvent can be an aqueous solution of an acid, for example diluted hydrochloric acid or tetrafluoroboric acid. The counter ion Y⁻ is defined by the acid used. Diazotation reactions are commonly used in organic synthesis, even on industrial scale, and are known by the person skilled in the art. In order to reduce the risk of decomposition of the intermediates of formula (XXV), the diazotation step and the cycloaddition steps can be performed sequentially, without the need of isolating (XXV). This variant is also described in Tetrahedron 2020, 76 (14), 131063 (Scheme 18).

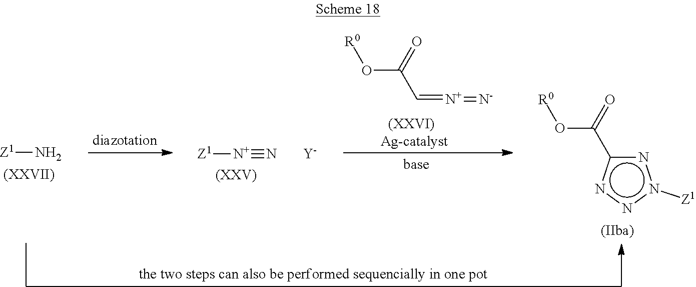

Compounds of formula (XXVII) are commercially available, as are compounds of formula (XXVI) (e.g. R⁹ is methyl, CAS [6832-16-2]).

Very similarly, compounds of formula (IIba), wherein A¹, A², A³ are N and Z¹ is as described in for formula (I), can be prepared by reacting a compound of formula (XXVIII):

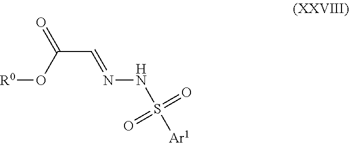

wherein R⁰ is C₁-C₆alkyl and Ar¹ is phenyl or p-tolyl, with compounds of formula (XXV), in the presence of a base, e.g., pyridine at temperatures from −50° C. to 50° C. to yield compounds of formula (IIba). Such reactions have good precedence in the literature, for example Chem. Comm. 2017, 53 (69), 9620-9623, Ang. Chem. Int. Ed. 2017, 56 (47), 15044-15048, and J. Am. Chem. Soc. 2016, 138 (44), 14609-14615. Compounds of formula (XXVIII) are prepared as described in the literature cited vide supra and exemplified in the preparation examples of the application.

Alternatively, compounds of formula (IIba), wherein A¹, A² and A³ are N and Z¹ is as defined in formula (I) can be obtained by coupling compounds of formula (XXIX),

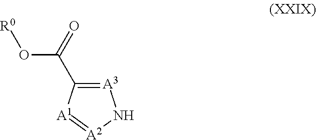

wherein A¹, A² and A³ are N, and R⁹ is as described vide supra, with a boronic acid derivative of formula (XXX),

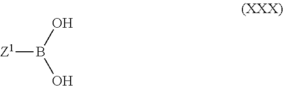

wherein Z¹ is as defined in formula I. This Chan-Lam type coupling reaction is usually performed in a solvent like dichloromethane, in presence of a catalytic amount of a copper-based catalyst under mild reaction temperatures, in presence of a base, such as potassium carbonate, under atmospheric air or oxygen. It is to be noted that compounds of formula (XXIX), wherein A¹, A² and A³ are N, exist in tautomeric forms, viz;

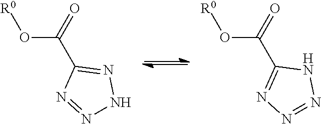

Persons skilled in the art will envisage that the coupling product of this reaction could be either one of the regioisomers or a mixture thereof, but when the reaction conditions are chosen as described in J. Org. Chem. 2014, 79, 6703-6707, the reaction shows an excellent regioselectivity for compounds of the formula (IIba). Those skilled in the art will realize that this Chan-Lam coupling is a general method for preparation of compounds of formula (IIb). Examples have been shown in the literature for compounds of formula (IIbb)

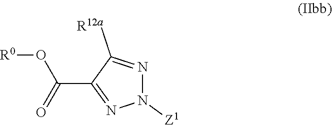

wherein Z¹, R^{12a}, and R⁹ are as previously defined (see J. Med. Chem. 2018, 61, 8, 3370-3388 and WO14/041106, 2014), compounds of formula (IIbc) and (IIbd)

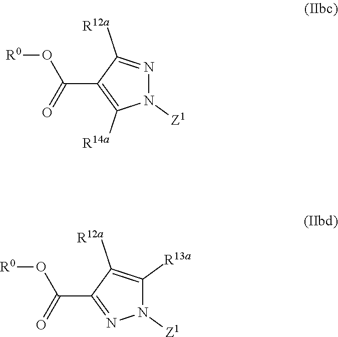

wherein Z¹, R^{12a}, R^{13a}, R^{14a}, and R⁹ are as defined under formula (I) (see WO15/155626, 2015, EP2390252, 2011) and compounds of formula (IIbe)

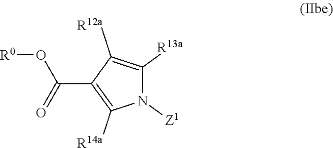

Wherein Z¹, R^{12a}, R^{13a}, R^{14a}, and R⁹ are as defined under formula (I) (see J. Med. Chem. 2017, 60 (14), 6166-6190, Org. Lett. 2008, 10 (8), 1653-1655, and Bio. Med. Chem. Lett. 2009, 19 (5), 1451-1456), as representative examples.

A further method for the preparation of compounds of formula (IIba), wherein A¹, A² and A³ are N and Z¹ are as defined in formula (I), is shown in Scheme 19.

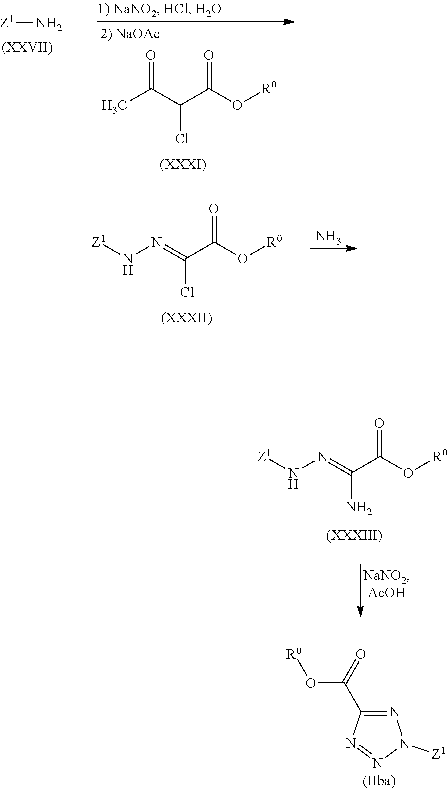

As shown in scheme 19, sequence begins with diazotation of compounds of formula (XXVII) as previously described vide supra and then treatment of the diazionium salt with a compound of formula (XXXI), wherein R⁹ is C₁-C₆alkyl, in the presence of in the presence of a mild base, for example sodium acetate to yield compounds of formula (XXXII). Compounds of formula (XXXII), wherein Z¹ and R⁰ are as previously defined, are then treated with aqueous ammonia in a miscible organic solvent, for example tetrahydrofuran or 2-methyl tetrahydrofuran, at temperatures between 0° C. to 30° C., to give compounds of formula (XXXIII). Finally, diazotation of compounds of formula (XXXIII) with a salt of nitrous acid, for example sodium nitrite, in a slightly acidic medium, for example acetic acid or aqueous hydrochloric acid at temperatures between −20° C. to 0° C. leads to spontaneous cyclization of the diazonium salt formed to the tetrazole compounds of formula (IIba). The sequence of reactions has been previously described in WO13/087805, 2013.

Compounds of formula (IIb) may also be prepared by alkylation of compounds of formula (XXIX)

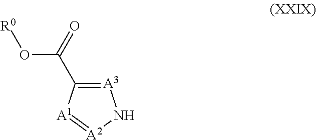

with compounds of formula (XXXIV)

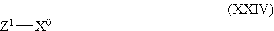

wherein Z¹ is as previously described under formula I and X⁰ is halogen, preferably chlorine, bromine or iodine in the presence of a base, for example and alkaline earth metal base such as NaOH, KOH, LiOH, Cs₂CO₃, K₂CO₃ and the like, in inert aprotic or protic solvents. Such alkylation's are well known to those skilled in the art and have been used in this context to prepare compounds of formula (IIb) as described for example in WO14/168221; WO10/043000; and WO14/32498. Those skilled in the art will realize this can lead to mixtures of regiosomeric compounds that can be separted by chromatographic techniques, or pure isomers can be obtained by the judicous choice of condtitions and additives (for example palladium catalysts for so called Buchwald aminations). For cases where Z¹ is heteraryl or aryl SnAr reactions (with or without copper catalysis) may be used to prepare compounds of formula (IIb) (see for example, Polyhedron 2019, 165, 22-30; U.S. Ser. No. 18/017,0909; Org. Lett. 2022, 24 (20), 3620-3625, J. Org. Chem. 2017, 82 (14), 7420-7427, Chem. Comm. 2021, 57 (57), 7047-7050, ACS Catalysis 2019, 9 (12), 10674-10679, Synthesis 2017, 49 (23), 5120-5130, J.org. Chem. 2019, 84 (12), 8160-8167, and references cited therein).

Salts of compounds of formula (I) may be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds (I), acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula (I), which have salt-forming properties, can be obtained in free form or in the form of salts.

The compounds of formula (I) and, where appropriate, the tautomer's thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, or diastereomer mixtures, depending on the number, absolute and relative configuration of asymmetric carbon atoms which occur in the molecule and/or depending on the configuration of non-aromatic double bonds which occur in the molecule, the invention relates to the pure isomers and also to all isomer mixtures which are possible and is to be understood in each case in this sense hereinabove and herein below, even when stereochemical details are not mentioned specifically in each case. Diastereomeric mixtures or racemic mixtures of compounds of formula (I), in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.

Enantiomeric mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is com-plexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents. Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

As an example, compounds with more than one asymmetric carbon atoms may exist in diastereomeric forms which can be optionally separated using for example supercritical fluid chromatography (SFC) chromatography with chiral columns. Such diastereomers can show a different fungicidal activity profile, but all isomers and diastereomers form part of this invention.

The compounds of formula (I) have three chiral carbon atoms, (three stereocenters, wherein the star (*) indicates the chiral carbon atom), such there are eight stereoisomers available. These eight stereoisomers 5 consist of four sets of enantiomers.

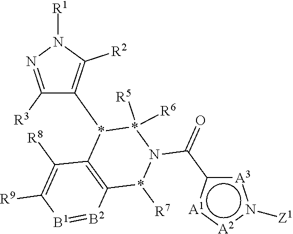

For compounds of formula (I) the relationship between enantiomers and diastereomers is illustrated in scheme 20.

A person skilled in the art is well aware that these diastereomers and enantiomers of formula (I) (as shown in scheme 20) wherein R¹, R², R³, R⁴, R⁵, R⁶, R⁷, R⁸, R⁹, B¹, B², A (A¹, A², A³) and Z¹ are as defined for formula (I) are within the scope if the invention.

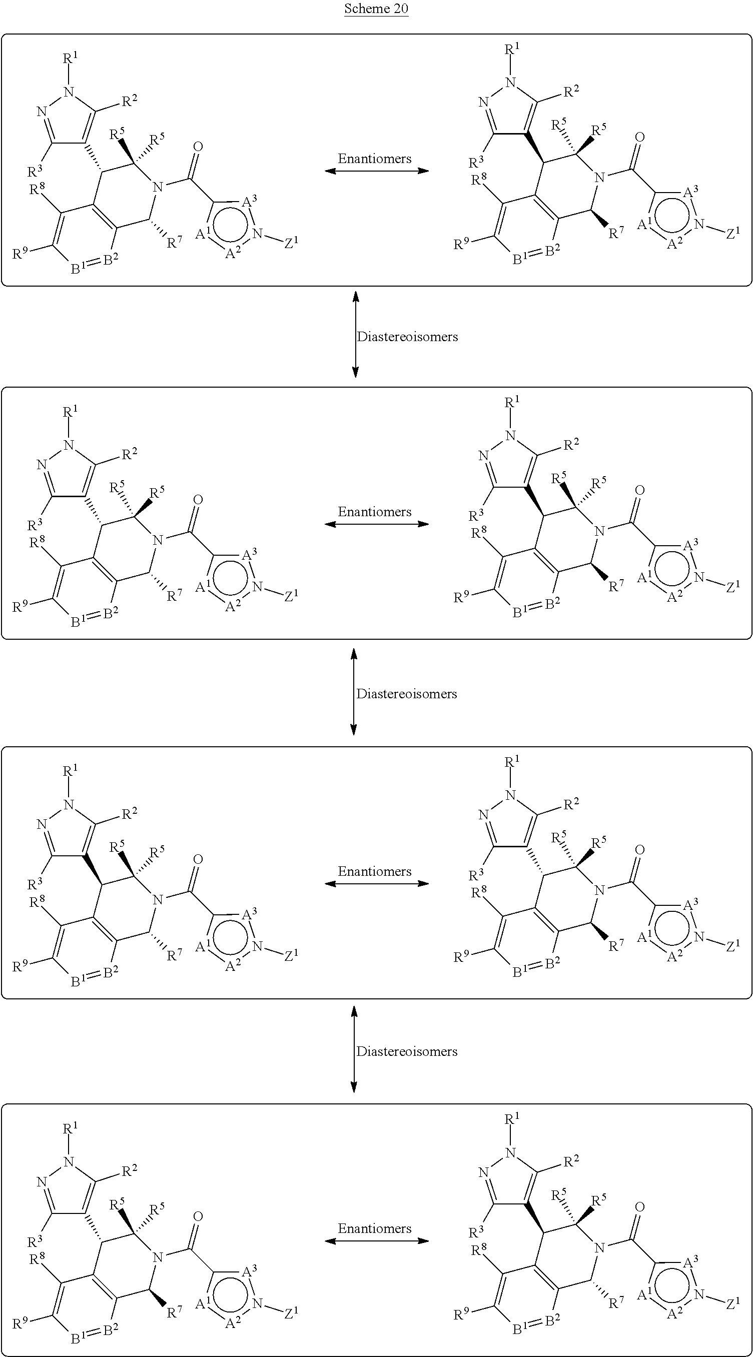

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

As already indicated, surprisingly, it has now been found that the compounds of formula (I) of the present invention have, for practical purposes, a very advantageous level of biological activity for protecting plants against diseases that are caused by fungi.

The compounds of formula (I) according to the invention can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) according to the invention is applied to the plants, to parts thereof or the locus thereof.

It is also possible to use a compound of formula (I) according to the invention as a fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of formula (I) according to the invention as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of formula (I) before planting: seed, for example, can be dressed before being sown. The active compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of formula (I) according to the invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula (I) according to the invention are for example, effective against fungi and fungal vectors of disease as well as phytopathogenic bacteria and viruses. These fungi and fungal vectors of disease as well as phytopathogenic bacteria and viruses are for example: Absidia corymbifera, Alternaria spp., Aphanomyces spp., Ascochyta spp., Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp., Cercospora spp. including C. arachidicola, Cercosporidium personatum, Cladosporium spp., Claviceps purpurea, Coccidioides immitis, Cochliobolus spp., Colletotrichum spp. including C. musae, Cryptococcus neoformans, Diaporthe spp., Didymella spp., Drechslera spp., Elsinoe spp.,Epidermophyton spp., Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp., Hemileia spp., Histoplasma spp. including H. capsulatum, Laetisaria fuciformis, Leptographium lindbergi, Leveillula taurica, Lophodermium seditiosum, Microdochium nivale, Microsporum spp., Monilinia spp., Mucor spp., Mycosphaerella spp. including M. graminicola, M. pomi, Oncobasidium theobromaeon, Ophiostoma piceae, Paracoccidioides spp., Penicillium spp. including P. digitatum, P. italicum, Petriellidium spp., Peronosclerospora spp. Including P. maydis, P. philippinensis and P. sorghi, Peronospora spp., Phaeosphaeria nodorum, Phakopsora pachyrhizi, Phellinus igniarus, Phialophora spp., Phoma spp., Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp., Pseudoperonospora spp. including P. cubensis, P. humuli, Pseudopeziza tracheiphila, Puccinia spp. including P. hordei, P. recondita, P. striiformis, P. triticina, Pyrenopeziza spp., Pyrenophora spp., Pyricularia spp. including P. oryzae, Pythium spp. including P. ultimum, Ramularia spp., Rhizoctonia spp., Rhizomucor pusillus, Rhizopus arrhizus, Rhynchosporium spp., Scedosporium spp. including S. apiospermum and S. prolificans, Schizothyrium pomi, Sclerotinia spp., Sclerotium spp., Septoria spp., including S. nodorum, S. tritici, Sphaerotheca macularis, Sphaerotheca fusca (Sphaerotheca fuliginea), Sporothorix spp., Stagonospora nodorum, Stemphylium spp., Stereum hirsutum, Thanatephorus cucumeris, Thielaviopsis basicola, Tilletia spp., Trichoderma spp. including T. harzianum, T. pseudokoningii, T. viride, Trichophyton spp., Typhula spp., Uncinula necator, Urocystis spp., Ustilago spp., Venturia spp. including V. inaequalis, Verticillium spp., and Xanthomonas spp.

The compounds of formula (I) according to the invention may be used for example on turf, ornamentals, such as flowers, shrubs, broad-leaved trees, or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA (b) toxin); YieldGard Rootworm® (maize variety that expresses a CryIIIB (b1) toxin); YieldGard Plus® (maize variety that expresses a CryIA (b) and a CryIIIB (b1) toxin); Starlink® (maize variety that expresses a Cry9 (c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33BR (cotton variety that expresses a CryIA (c) toxin); Bollgard I® (cotton variety that expresses a CryIA (c) toxin); Bollgard II® (cotton variety that expresses a CryIA (c) and a CryllA (b) toxin); VIPCOT® (cotton variety that expresses a VIP toxin); NewLeaf® (potato variety that expresses a CryIIIA toxin); Nature-Gard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed using recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as-endotoxins, e.g.

Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp, or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by delta-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A⁰⁵⁵, a cathepsin-G-recognition sequence is inserted into a Cry3A toxin (see WO2003/018810).

Examples of such toxins or transgenic plants capable of synthesising such toxins are disclosed, for example, in EP-0374753, WO93/07278, WO95/34656, EP0427529, EP0451878 and WO03/052073.

The processes for the preparation of such transgenic plants are generally known to a person skilled in the art and are described, for example, in the publications mentioned above. CryI-type deoxyribonucleic acids and their preparation are known, for example, from WO95/34656, EP0367474, EP0401979 and WO90/13651.

The toxin contained in the transgenic plants imparts to the plants tolerance to harmful insects. Such insects can occur in any taxonomic group of insects but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33BR (cotton variety that expresses a Cry1Ac toxin); Bollgard I® (cotton variety that expresses a

Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l′Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l′Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l′Hobit 27, F-31 790 St. Sauveur, France, registration number C/FR/96/05/10. Maize which has been rendered insect-resistant by transgenic expression of a modified Cry3A toxin. This toxin is Cry3A⁰⁵⁵ modified by insertion of a cathepsin-G-protease recognition sequence. The preparation of such transgenic maize plants is described in WO2003/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B 1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The compounds of formula (I) according to the invention may be used in controlling or preventing phytopathogenic diseases, especially phytopathogenic fungi such as Alternaria species in fruits, vegetables and potatoes; Botrytis cinerea in strawberries, tomatoes, sunflower, pulse crops, vegetables and grapes; Rhizoctonia solani in potatoes and vegetables; Uncinula necator in grapes; Cladosporium cucumerinum, Didymella bryoniae, Sphaerotheca fuliginea and Glomerella lagenarium in cucurbits; Leveillula taurica in cucurbits and solanacious crops; Fusarium spp. in cereals; Leptosphaeria spp. in cereals; and Zymospetoria spp. in cereals.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of formula (I) according to the invention may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g., in polymeric substances. As with the type of the compositions, the methods of application, such as spraying, atomising, dusting, scattering, coating, or pouring, are chosen in accordance with the intended objectives and the prevailing circumstances. The compositions may also contain further adjuvants such as stabilizers, antifoams, viscosity regulators, binders, or tackifiers, as well as fertilizers, micronutrient donors, or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders, or fertilizers. Such carriers are for example described in WO1997/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely divided form as a result of vaporisation of a low boiling dispersant solvent carrier, may also be used.

Suitable agricultural adjuvants and carriers that are useful in formulating the compositions of the invention in the formulation types described above are well known to a person skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaceous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface-active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2 ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants, and sticking agents.

In addition, further, other biocidal active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidal active ingredients may be fungicides, herbicides, insecticides, bactericides, acaricides, nematicides, and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, United States Patent No. U.S. Pat. No. 6,919,298 and include, for example, salicylates and the commercial SAR inducer acibenzolar-S-methyl.

The compounds of formula (I) according to the invention are normally used in the form of agrochemical compositions and can be applied to the crop area or plant to be treated, simultaneously or in succession with further compounds. These further compounds can be e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) according to the invention may be used in the form of (fungicidal) compositions for controlling or protecting against phytopathogenic microorganisms, comprising as active ingredient at least one compound of formula (I) or of at least one preferred individual compound as defined herein, in free form or in agrochemical usable salt form, and at least one of the above-mentioned adjuvants.

The invention therefore provides a composition, preferably a fungicidal composition, comprising at least one compound of formula (I) according to the invention, an agriculturally acceptable carrier and optionally an adjuvant. An agricultural acceptable carrier is for example a carrier that is suitable for agricultural use. Agricultural carriers are well known in the art. Preferably, said composition may comprise at least one or more pesticidal-active compounds, for example an additional fungicidal active ingredient in addition to the compound of formula (I).

The compound of formula (I) according to the invention may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following: acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients include the following: petroleum oils, 1,1-bis(4-chlorophenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxa-fos, benzyl benzoate, bixafen, brofenvalerate, bromocyclen, bromophos, bromopropylate, buprofezin, butocarboxim, butoxycarboxim, butylpyridaben, calcium polysulfide, camphechlor, carbanolate, carbophenothion, cymiazole, chinomethionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dinopenton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fenpyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, oxolinic potassium octhilinone, acid, oxytetracycline, hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8, 10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B¹, trimedlure B², trimedlure C, trunc-call, 2-(octylthio) ethanol, butopyronoxyl, butoxy (polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl) ethane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy) ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethylcarbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, El 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla, schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothiophene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin, 2-(2-butoxyethoxy)ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil, imibenconazole, ipconazole, metconazole, myclobutanil, paclobutrazole, pefurazoate, penconazole, prothioconazole, pyrifenox, prochloraz, propiconazole, pyrisoxazole, simeconazole, tebuconazole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline, procymidone, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chlorothalonil, cyflufenamid, cymoxanil, cyclobutrifluram, diclocymet, diclomezine, dicloran, diethofencarb, dimethomorph, flumorph, dithianon, ethaboxam, etridiazole, famoxadone, fenamidone, fenoxanil, ferimzone, fluazinam, flumetylsulforim, fluopicolide, fluoxytioconazole, flusulfamide, fluxapyroxad, fenhexamid, fosetylaluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino [1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1,3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl)quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl) phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, metarylpicoxamid, tebufloquin, ipflufenoquin, quinofumelin, isofetamid, ethyl 1-[[4-[[2-(trifluoromethyl)-1,3-dioxolan-2-yl]methoxy]phenyl]methyl]pyrazole-3-carboxylate (may be prepared from the methods described in WO2020/056090), ethyl 1-[[4-[(Z)-2-ethoxy-3,3,3-trifluoro-prop-1-enoxy]phenyl]methyl]pyrazole-3-carboxylate (may be prepared from the methods described in WO2020/056090), methyl N-[[4-[1-(4-cyclopropyl-2,6-difluoro-phenyl)pyrazol-4-yl]-2-methyl-phenyl]methyl]carbamate (may be prepared from the methods described in WO2020/097012), methyl N-[[4-[1-(2,6-difluoro-4-isopropyl-phenyl)pyrazol-4-yl]-2-methyl-phenyl]methyl]carbamate (may be prepared from the methods described in WO2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO2020/109391), N-[2-[2,4-dichloro-phenoxy]phenyl]-3-(difluoromethyl)-1-methyl-pyrazole-4-carboxamide, N-[2-[2-chloro-4-(trifluoromethyl) phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flufenoxadiazam, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1,1,3-trimethylindan-4-yl)pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, a -(1,1-dimethylethyl)-α-[4′-(trifluoromethoxy) [1,1′-biphenyl]-4-yl]-5-pyrimidinemethanol, fluoxapiprolin, enoxastrobin, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl) phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl) phenoxy]prop-2-enoate, methyl (Z)-3-methoxy-2-[2-methyl-5-[3-(trifluoromethyl)pyrazol-1-yl]phenoxy]prop-2-enoate (these compounds may be prepared from the methods described in WO2020/079111), methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, seboctylamine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl) oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-N-((1S)-1-benzyl-3-chloro-1-methyl-but-3-enyl)-8-fluoro-quinoline-3-carboxamide quinoline-3-carboxamide, (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl) isoquinoline, 4,4-difluoro-3,3-dimethyl-1-(7-methylpyrazolo[1,5-a]pyridin-3-yl) isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl) isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta [e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N, N-dimethyl-1-[[4-[5-(trifluoromethyl)-1,2,4-oxadiazol-3-yl]phenyl]methyl]-1,2,4-triazol-3-amine (these compounds may be prepared from the methods described in WO2017/055473, WO2017/055469, WO2017/093348 and WO2017/118689); 2-[6-(4-chlorophenoxy)-2-(trifluoromethyl)-3-pyridyl]-1-(1,2,4-triazol-1-yl)propan-2-ol (this compound may be prepared from the methods described in WO2017/029179); 2-[6-(4-bromophenoxy)-2-(trifluoromethyl)-3-pyridyl]-1-(1,2,4-triazol-1-yl)propan-2-ol (this compound may be prepared from the methods described in WO2017/029179); 3-[2-(1-chlorocyclopropyl)-3-(2-fluorophenyl)-2-hydroxy-propyl]imidazole-4-carbonitrile (this compound may be prepared from the methods described in WO2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO2016/156290); (4-phenoxyphenyl)methyl 2-amino-6-methyl-pyridine-3-carboxylate (this compound may be prepared from the methods described in WO2014/006945); 2,6-Dimethyl-1H,5H-[1,4]dithiino[2,3-c: 5,6-c′]dipyrrole-1,3,5,7 (2H,6H)-tetrone (this compound may be prepared from the methods described in WO2011/138281) N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N, 3-dimethyl-pent-3-enamide (this compound may be prepared from the methods described in WO2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO2016/202742); 2-(difluoromethyl)-N-[(3S)-3-ethyl-1,1-dimethyl-indan-4-yl]pyridine-3-carboxamide (this compound may be prepared from the methods described in WO2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO2017/220485); 2-oxo-N-propyl-2-[4-[5-(trifluoromethyl)-1,2,4-oxadiazol-3-yl]phenyl]acetamide (this compound may be prepared from the methods described in WO2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO2018/158365); 2,2-difluoro-N-methyl-2-[4-[5-(trifluoromethyl)-1,2,4-oxadiazol-3-yl]phenyl]acetamide, N—[(E)-methoxyiminomethyl]-4-[5-N—[(Z)-methoxyiminomethyl]-4-[5-(trifluoromethyl)-1,2,4-(trifluoromethyl)-1,2,4-oxadiazol-3-yl]benzamide, oxadiazol-3-yl]benzamide, N—[N-methoxy-methyl-carbonimidoyl]-4-[5-(trifluoromethyl)-1,2,4-oxadiazol-3-yl]benzamide (these compounds may be prepared from the methods described in WO2018/202428). The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP0357460, EP0444964 and EP0594291. Additional anthelmintic agents include semisynthetic and biosynthetic avermectin/milbemycin derivatives such as those described in U.S. Pat. No. 5,015,630, WO9415944 and WO9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The compounds of the invention may be used in combination with derivatives and analogues of the general class of dioxomorpholine antiparasitic agents as described in WO9615121 and also with anthelmintic active cyclic depsipeptides such as those described in WO9611945, WO9319053, WO9325543, EP0626375, EP0382173, WO9419334, EP0382173, and EP0503538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in WO95/19363 or WO04/72086, particularly the compounds disclosed therein.

Other examples of such biologically active compounds that the compounds of the invention may be used in combination with include but are not restricted to the following:

Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl) carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a -cypermethrin, beta-cypermethrin, bioallethrin, bioallethrin ((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195, RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp. aizawai, kurstaki, Bacillus thuringiensis delta endotoxin, baculovirus, entomopathogenic bacteria, virus, and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The following mixtures of the compounds of Formula (I) with active ingredients are preferred. The abbreviation “TX” means one compound selected from compounds of formula (I), (II), (II-A), (II-A¹), (II-B), (II-B1), (II-C), (II-C₁), (II-D), or (II-D1) according to the present invention, or compounds selected from compounds listed in Tables A-1 to A-24, or Table P (below):
a compound selected from the group of substances consisting of petroleum oils+TX, 1,1-bis(4-chlorophenyl)-2-ethoxyethanol+TX, 2,4-dichlorophenyl benzenesulfonate+TX, 2-fluoro-N-methyl-N-1-naphthylacetamide +TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxafos+TX, benzyl benzoate+TX, bixafen+TX, brofenvalerate+TX, bromocyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chinomethionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin |+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dinopenton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fenpyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure |+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B₂+TX, trimedlure C+TX, trunc-call+TX, 2-(octylthio) ethanol+TX, butopyronoxyl+TX, butoxy (polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl) ethane+TX, 1,2-dichloropropane with 1,3-dichloropropene+TX, 1-bromo-2-chloroethane+TX, 2,2,2-trichloro-1-(3,4-dichlorophenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy) ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethylcarbamate+TX, 4-methyl(prop-2-ynyl)amino-3,5-xylyl+methylcarbamate TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′, O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene |+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla)+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, and meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothiophene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, 2-fluoro-N-(3-methoxyphenyl)-9H-purin-6-amine+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, 2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesamolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, mercuric oxide+TX, thiophanate-methyl+TX, azaconazole+TX, bitertanol+TX, bromuconazole+TX, cyproconazole+TX, difenoconazole+TX, diniconazole+TX, epoxiconazole+TX, fenbuconazole+TX, fluquinconazole+TX, flusilazole+TX, flutriafol+TX, furametpyr+TX, hexaconazole+TX, imazalil+TX, imibenconazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebuconazole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl-+TX, Rmetalaxyl+TX, ofurace+TX, oxadixyl+TX, carbendazim+TX, debacarb+TX, fuberidazole+TX, thiabendazole+TX, chlozolinate+TX, dichlozoline+TX, myclozoline+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb+TX, chlorothalonil+TX, cyflufenamid+TX, cymoxanil+TX, cyclobutrifluram+TX, diclocymet+TX, diclomezine+TX, dicloran+TX, diethofencarb+TX, dimethomorph+TX, flumorph+TX, dithianon+TX, ethaboxam+TX, etridiazole+TX, famoxadone+TX, fenamidone+TX, fenoxanil+TX, ferimzone+TX, fluazinam+TX, fluopicolide+TX, flusulfamide+TX, fluxapyroxad+TX, fenhexamid+TX, fosetyl-aluminium+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino [1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl) N-(2-chloro-6-fluorophenyl)-1,3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[[1-(4-chlorophenyl)pyrazol-3-yl]oxymethyl]-3-methyl-phenyl]-4-methyl-tetrazol-5-one+TX, 1-methyl-4-[3-methyl-2-[[[2-methyl-4-(3,4,5-trimethylpyrazol-1-yl) phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, tebufloquin+TX, ipflufenoquin+TX, quinofumelin+TX, isofetamid+TX, N-[2-[2,4-dichloro-phenoxy]phenyl]-3-(difluoromethyl)-1-methyl-pyrazole-4-carboxamide+TX, N-[2-[2-chloro-4-(trifluoromethyl) phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1,1,3-trimethylindan-4-yl)pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, α-(1,1-dimethylethyl)-α-[4′-(trifluoromethoxy) [1,1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+trinexapac TX, +TX, coumoxystrobin+TX, zhongshengmycin+TX, thiodiazole copper+TX, zinc thiazole+TX, amectotractin+TX, iprodione+TX, N-octyl-N′-[2-(octylamino)ethyl]ethane-1,2-diamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[2-trifluoromethyl) oxetan-2-yl]phenyl]-N-methyl-formamidine+TX, N-ethyl-N′-[5-methoxy-2-methyl-4-[2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl) isoquinoline+TX, 4,4-difluoro-3,3-dimethyl-1-(7-methylpyrazolo[1,5-a]pyridin-3-yl) isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl) isoquinoline+4,4-difluoro-1-(5-fluoro-4-methyl-benzimidazol-1-yl)-3,3-TX, dimethyl-isoquinoline+TX, 3-(4,4-difluoro-3,3-dimethyl-1-isoquinolyl)-7,8-dihydro-6H-cyclopenta [e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO2017/055473, WO2017/055469, WO2017/093348 and WO2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO2017/029179); 3-[2-(1-chlorocyclopropyl)-3-(2-fluorophenyl)-2-hydroxy-propyl]imidazole-4-carbonitrile+TX (this compound may be prepared from the methods described in WO2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO2016/156290); (4-phenoxyphenyl)methyl 2-amino-6-methyl-pyridine-3-carboxylate+TX (this compound may be prepared from described in WO2014/006945); 2,6-Dimethyl-1H,5H-[1,4]dithiino[2,3-c: 5,6-c′]dipyrrole—the methods 1,3,5,7 (2H,6H)-tetrone+TX (this compound may be prepared from the methods described in WO2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N, 3-dimethyl-pent-3-enamide+TX (this compound may be prepared from the methods described in WO2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO2016/202742); 2-(difluoromethyl)-N-[(3S)-3-ethyl-1,1-dimethyl-indan-4-yl]pyridine-3-carboxamide+TX (this compound may be prepared from the methods described in WO2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO2017/220485); 2-oxo-N-propyl-2-[4-[5-(trifluoromethyl)-1,2,4-oxadiazol-3-yl]phenyl]acetamide+TX (this compound may be prepared from the methods described in WO2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO2018/158365); 2,2-difluoro-N-methyl-2-[4-[5-(trifluoromethyl)-1,2,4-oxadiazol-3-yl]phenyl]acetamide+TX, N—[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO2018/202428), chloroinconazide+TX, flumetylsulforim+TX, fluoxytioconazole+TX, flufenoxadiazam+TX, metarylpicoxamid+TX.

The references in brackets behind the active ingredients, e.g. [3878-19-1]refer to the Chemical Abstracts Registry number. The above described mixing partners are known. Where the active ingredients are included in “The Pesticide Manual” [The Pesticide Manual-A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright @ 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compound selected from compounds of formula (I), (II), (II-A), (II-A¹), (II-B), (II-B1), (II-C), (II-C₁), (II-D), or (II-D1) according to the present invention, or compounds selected from compounds listed in Tables A-1 to A-24, or Table P (below), is preferably in a mixing ratio of from 100:1 to 1:100, especially from 50:1 to 1:50, more especially in a ratio of from 20:1 to 1:20, even more especially from 10:1 to 1:10, and still more especially from 5:1 to 1:5 Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound selected from compounds of formula (I), (II), (II-A), (II-A¹), (II-B), (II-B1), (II-C), (II-C₁), (II-D), or (II-D1) according to the present invention, or compounds selected from compounds listed in Tables A-1 to A-24, or Table P (below), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying a compound selected from compounds of formula (I), (II), (II-A), (II-A¹), (II-B), (II-B1), (II-C), (II-C₁), (II-D), or (II-D1) according to the present invention, or compounds selected from compounds listed in Tables A-1 to A-24, or Table P (below), and the active ingredient(s) as described above, is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of the invention is related to the use of a compound of formula (I) according to the invention or of a preferred individual compound as defined herein, of a composition comprising at least one compound of formula (I) or at least one preferred individual compound as defined herein, or of a fungicidal or insecticidal mixture comprising at least one compound of formula (I) or at least one preferred individual compound as defined herein, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula (I) according to the invention or of a preferred individual compound as defined herein as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I) according to the invention, or an agrochemical composition which contains at least one compound of formula (I), is foliar application. The frequency of application and the rate of application will depend on the risk of infestation by the corresponding pathogen or insect. However, the compounds of formula (I) according to the invention can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula (I) may also be applied to seeds (coating) by impregnating the seeds or tubers either with a liquid formulation of the fungicide or coating them with a solid formulation.

A formulation, e.g., a composition containing the compound of formula (I) according to the invention and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

The term “g a.i./ha” as used herein refer to the application rate given in gram [g] of active ingredient [a.i.]per unit of surface [ha]. The unit hectare (symbol ha) is the metric unit of area that equals a square with 100 m side (1 hm²) or 10,000 square meters. Hectare is a commonly used unit of area in the metric system.

When the combinations of the present invention are used for treating seed, rates of 0.001 to 50 g of a compound of formula (I) per kg of seed, preferably from 0.01 to 10 g per kg of seed are generally sufficient.

Suitably, a composition comprising a compound of formula (I) according to the present invention is applied either preventative, meaning prior to disease development or curative, meaning after disease development. The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g., by mixing the active ingredients with appropriate formulation inerts (diluents, solvents, fillers and optionally other formulating ingredients such as surfactants, biocides, anti-freeze, stickers, thickeners and compounds that provide adjuvancy effects). Also, conventional slow-release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g., as an aqueous suspension or in a dry powder form having good adherence to the seeds. Such seed dressing formulations are known in the art.

Seed dressing formulations may contain the single active ingredients or the combination of active ingredients in encapsulated form, e.g., as slow-release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) according to the invention optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline, compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The disclosure in the present application makes available each and every combination of embodiments disclosed herein.

The compounds according to the following Tables A-1 to A-24 may be prepared according to the methods described above. The examples which follow are intended to illustrate the invention and show preferred compounds of formula (I). In any of Tables A-1 to A-24 below, the presence of one or more possible asymmetric carbon atoms in a compound of formula (I) according to the invention means that the compounds may occur in chiral isomeric forms, i.e., enantiomeric or diastereomeric forms.

Table A: This table discloses 20 substituent definitions (G) of compounds of the formula (Ia) according to the invention:

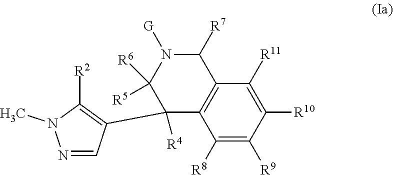

wherein G according to compounds of Formula (I) of the invention is

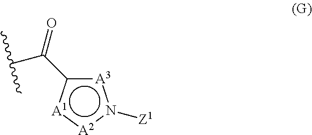

and wherein the G substituents are as defined below:

| Index | G |
|---|---|
| 1 | [2-(2,4-difluorophenyl)tetrazole-5-carbonyl] |
| 2 | [2-(2,4-difluorophenyl)triazole-4-carbonyl] |
| 3 | [1-(2,4-difluorophenyl)triazole-4-carbonyl] |
| 4 | [2-(3,5-difluoro-2-pyridyl)tetrazole-5- |
| | carbonyl] |
| 5 | [2-(3,5-difluoro-2-pyridyl)triazole-4-carbonyl] |
| 6 | [1-(3,5-difluoro-2-pyridyl)triazole-4-carbonyl] |
| 7 | [2-(4-fluorophenyl)tetrazole-5-carbonyl] |
| 8 | [2-(5-fluoro-2-pyridyl)tetrazole-5-carbonyl] |
| 9 | [2-(4-fluorophenyl)triazole-4-carbonyl] |
| 10 | [1-(4-fluorophenyl)triazole-4-carbonyl] |
| 11 | [2-(2-fluorophenyl)tetrazole-5-carbonyl] |
| 12 | [2-(2-fluorophenyl)triazole-4-carbonyl] |
| 13 | [1-(2-fluorophenyl)triazole-4-carbonyl] |
| 14 | (2-phenyltetrazole-5-carbonyl) |
| 15 | (2-phenyltriazole-4-carbonyl) |
| 16 | (1-phenyltriazole-4-carbonyl) |
| 17 | [1-(2,4-difluorophenyl)pyrazole-4-carbonyl] |
| 18 | [1-(2,4-difluorophenyl)pyrazole-3-carbonyl] |
| 19 | [1-(2,4-difluorophenyl)-1,2,4-triazole-3-carbonyl] |
| 20 | [1-(2,4-difluorophenyl)pyrrole-3-carbonyl] |

The following compounds are thus specifically described in Tables A-1 to A-24 with the substituents for Formula (Ia)

Table A-1: This table provides 20 compounds (A-1.01) to (A-1.20) of formula (Ia) wherein R², R⁴, R⁵, R⁶, R⁷, R⁸, R⁹, R¹⁰ and R¹¹ are H, and G substituents are as defined in Table A. For example, compound (A-1.01) has the following structure:

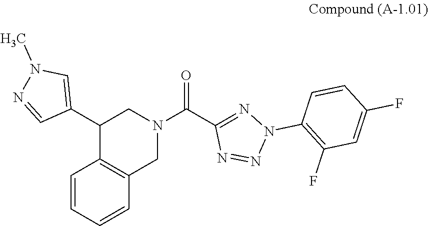 [SMILES: Cn1cc(C2CN(C(=O)c3nnn(-c4ccc(F)cc4F)n3)Cc3ccccc32)cn1]

Table A-2: This table provides 20 compounds (A-2.01) to (A-2.20) of formula (Ia) wherein R², R⁵, R⁶, R⁷, R⁸, R⁹, R¹⁰ and R¹¹ are H, R² is CH₃, and G substituents are as defined in Table A. For example, compound (A-2.09) has the following structure:

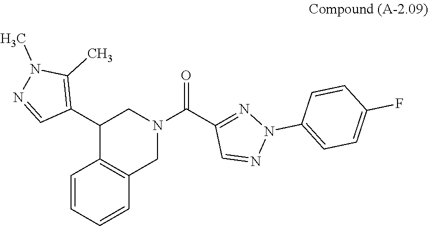 [SMILES: Cc1c(C2CN(C(=O)c3cnn(-c4ccc(F)cc4)n3)Cc3ccccc32)cnn1C]

Table A-3: This table provides 20 compounds (A-3.01) to (A-3.20) of formula (Ia) wherein R⁴, R⁵, R⁶, R⁷, R⁸, R⁹, R¹⁰ and R¹¹ are H, R² is Cl, and G substituents are as defined in Table A.

Table A-4: This table provides 20 compounds (A-4.01) to (A-4.20) of formula (Ia) wherein R², R⁵, R⁶, R⁷, R⁸, R⁹, R¹⁰ and R¹¹ are H, R⁴ is CH₃, and G substituents are as defined in Table A. For example, compound (A-4.06) has the following structure:

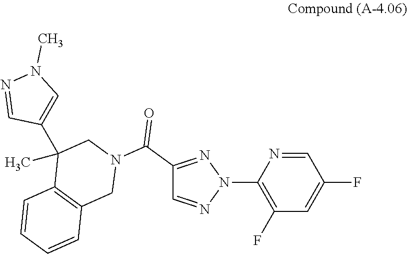 [SMILES: Cn1cc(C2(C)CN(C(=O)c3cnn(-c4ncc(F)cc4F)n3)Cc3ccccc32)cn1]

Table A-5: This table provides 20 compounds (A-5.01) to (A-5.20) of formula (Ia) wherein R⁵, R⁶, R⁷, R⁸, R°, R¹⁰ and R¹¹ are H, R² and R⁴ are CH₃, and G substituents are as defined in Table A.

Table A-6: This table provides 20 compounds (A-6.01) to (A-6.20) of formula (Ia) wherein R⁵, R⁶, R⁷, R⁸, R⁹, R¹⁰ and R¹¹ are H, R² is Cl, R⁴ is CH₃, and G substituents are as defined in Table A.

Table A-7: This table provides 20 compounds racemic-syn-(A-7.01) to racemic-syn-(A-7.20) of formula (Ia) wherein R², R⁴, R⁵, R⁶, R⁸, R⁹, R¹⁰ and R¹¹ are H, R⁷ is CH₃, and G substituents are as defined in Table A. All the compounds in this table have a syn relationship of the R⁷ substituent with the pyrazole substituent. For example, compound racemic-syn-(A-7.06) has the following structure:

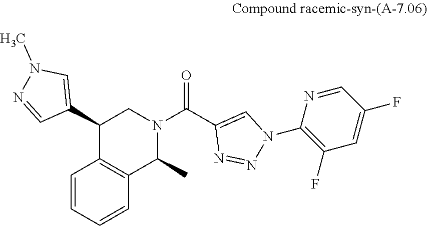 [SMILES: C[C@H]1c2ccccc2[C@@H](c2cnn(C)c2)CN1C(=O)c1cn(-c2ncc(F)cc2F)nn1]

Table A-8: This table provides 20 compounds racemic-syn-(A-8.01) to racemic-syn-(A-8.020) of formula (Ia) wherein R⁴, R⁵, R⁶, R⁷, R⁸, R⁹, R¹⁰ and R¹¹ are H, R² and R⁷ are CH₃, and G substituents are as defined in Table A. All the compounds in this table have a syn relationship of the R⁷ substituent with the pyrazole substituent. For example, compound racemic-syn-(A-8.01) has the following structure:

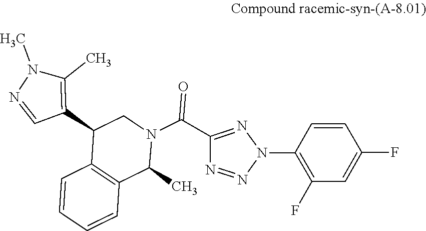 [SMILES: Cc1c([C@H]2CN(C(=O)c3nnn(-c4ccc(F)cc4F)n3)[C@@H](C)c3ccccc32)cnn1C]

Table A-9: This table provides 20 compounds racemic-syn-(A-9.01) to racemic-syn-(A-9.20) of formula (Ia) wherein R⁴, R⁵, R⁶, R⁸, R⁹, R¹⁰ and R¹¹ are H, R² is Cl and R⁷ are CH₃, and G substituents are as defined in Table A. All the compounds in this table have a syn relationship of the R⁷ substituent with the pyrazole substituent. For example, compound (A-9.14) has the following structure:

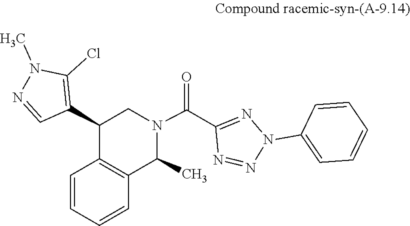 [SMILES: C[C@H]1c2ccccc2[C@@H](c2cnn(C)c2Cl)CN1C(=O)c1nnn(-c2ccccc2)n1]

Table A-10: This table provides 6 compounds (A-10.01) to (A-10.06), of formula (Ia) wherein R², R⁴, R⁵, R⁶, R⁷, R⁸, R⁹, and R¹⁰ are H, R¹¹ is Br, and G substituents are as defined in Table A.

Table A-11: This table provides 6 compounds (A-11.01) to (A-11.06) of formula (Ia) wherein R², R⁴, R⁵, R⁶, R⁷, R⁸, R⁹, and R¹⁰ are H, R¹¹ is Cl, and G substituents are as defined in Table A.

Table A-12: This table provides 6 compounds (A-12.01) to (A-12.06) of formula (Ia) wherein R², R⁴, R⁵, R⁶, R⁷, R⁸, R⁹, and R¹⁰ are H, R¹¹ is CN, and G substituents are as defined in Table A.

Table A-13: This table provides 6 compounds (A-13.01) to (A-13.06), of formula (Ia) wherein R², R⁵, R⁶, R⁷, R⁸, R⁹, and R¹⁰ are H, R⁴ is CH₃, R¹¹ is Br, and G substituents are as defined in Table A.

Table A-14: This table provides 6 compounds (A-14.01) to (A-14.06), of formula (Ia) wherein R², R⁵, R⁶, R⁷, R⁸, R⁹, and R¹⁰ are H, R⁴ is CH₃, R¹¹ is Cl, and G substituents are as defined in Table A.

Table A-15: This table provides 6 compounds (A-15.01) to (A-15.06), of formula (Ia) wherein R², R⁵, R⁶, R⁷, R⁸, R⁹, and R¹⁰ are H, R⁴ is CH₃, R¹¹ is CN, and G substituents are as defined in Table A.

Table A-16: This table provides 6 compounds racemic-syn-(A-16.01) to racemic-syn-(A-16.006) of formula (Ia) wherein R², R⁴, R⁵, R⁶, R⁸, R⁹, and R¹⁰ are H, R⁷ is CH₃, R¹¹ is Br, and G substituents are as defined in Table A. All the compounds in this table have a syn relationship of the R⁷ substituent with the pyrazole substituent. For example, compound racemic-syn-(A-16.04) has the following structure:

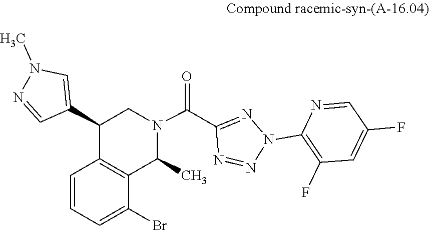 [SMILES: C[C@H]1c2c(Br)cccc2[C@@H](c2cnn(C)c2)CN1C(=O)c1nnn(-c2ncc(F)cc2F)n1]

Table A-17: This table provides 6 compounds racemic-syn-(A-17.01) to racemic-syn-(A-17.006) of formula (Ia) wherein R², R⁴, R⁵, R⁶, R⁸, R⁹, and R¹⁰ are H, R⁷ is CH₃, R¹¹ is Cl, and G substituents are as defined in Table A. All the compounds in this table have a syn relationship of the R⁷ substituent with the pyrazole substituent.

Table A-18: This table provides 6 compounds racemic-syn-(A-18.01) to racemic-syn-(A-18.006) of formula (Ia) wherein R², R⁴, R⁵, R⁶, R⁸, R⁹, and R¹⁰ are H, R⁷ is CH₃, R¹¹ is CN, and G substituents are as defined in Table A. All the compounds in this table have a syn relationship of the R⁷ substituent with the pyrazole substituent.

Table A-19: This table provides 6 compounds racemic-syn-(A-19.01) to racemic-syn-(A-19.006) of formula (Ia) wherein R⁴, R⁵, R⁶, R⁸, R⁹, and R¹⁰ are H, R² and R⁷ are CH₃, R¹¹ is Br, and G substituents are as defined in Table A. All the compounds in this table have a syn relationship of the R⁷ substituent with the pyrazole substituent.

Table A-20: This table provides 6 compounds racemic-syn-(A-20.01) to racemic-syn-(A-20.006) of formula (Ia) wherein R⁴, R⁵, R⁶, R⁸, R⁹, and R¹⁰ are H, R² and R⁷ are CH₃, R¹¹ is Cl, and G substituents are as defined in Table A. All the compounds in this table have a syn relationship of the R⁷ substituent with the pyrazole substituent.

Table A-21: This table provides 6 compounds racemic-syn-(A-21.01) to racemic-syn-(A-21.006) of formula (Ia) wherein R⁴, R⁵, R⁶, R⁸, R⁹, and R¹⁰ are H, R² and R⁷ are CH₃, R¹¹ is CN, and G substituents are as defined in Table A. All the compounds in this table have a syn relationship of the R⁷ substituent with the pyrazole substituent. For example, compound racemic-syn-(A-21.01) has the following structure:

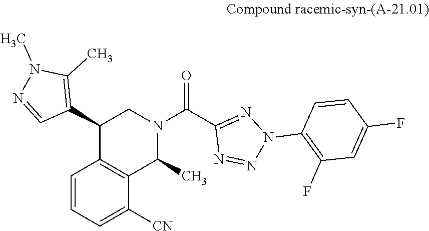 [SMILES: Cc1c([C@H]2CN(C(=O)c3nnn(-c4ccc(F)cc4F)n3)[C@@H](C)c3c(C#N)cccc32)cnn1C]

Table A-22: This table provides 3 compounds (A-22.01) to (A-22.03) of formula (Ia) wherein R⁴, R⁵, R⁶, R⁷, R⁸, R¹⁰ and R¹¹ are H, R² is CH₃, R⁹ is Br, and G substituents are as defined in Table A.

Table A-23: This table provides 3 compounds (A-23.01) to (A-23.03) of formula (Ia) wherein R⁴, R⁵, R⁶, R⁷, R⁸, R¹⁰ and R¹¹ are H, R² is CH₃, R⁹ is OCH₃, and G substituents are as defined in Table A.

Table A-24: This table provides 3 compounds (A-24.01) to (A-24.03) of formula (Ia) wherein R⁴, R⁵, R⁶, R⁷, R⁸, R¹⁰ and R¹¹ are H, R² is CH₃, R⁹ is CN, and G substituents are as defined in Table A.

### EXAMPLES

The Examples which follow serve to illustrate the invention and are not meant in any way to limit the invention.

The compounds of the invention can be distinguished from known compounds by virtue of greater efficacy at low application rates, which can be verified by a person skilled in the art using the experimental procedures outlined in the Examples, using lower application rates if necessary, for example 60 ppm, 20 ppm or 2 ppm.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius and “m.p.” means melting point. LC/MS means Liquid Chromatography Mass Spectroscopy and the description of the apparatus, and the methods is as follows.

¹H NMR and ¹⁹F NMR measurements were recorded on a Bruker 400 MHz spectrometer, chemical shifts are given in ppm relevant to a TMS (1H) and CFCl3 (¹⁹F) standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

LC-MS Method A: Spectra were recorded on a Mass Spectrometer from Waters Corporation (SQD, SQDII or QDA Single quadrupole mass spectrometer) equipped with an electrospray source (Polarity: positive and negative ions), Capillary: 0.8-3.00 KV, Cone: 5-30 V, Source Temperature: 120-150° C., Desolvation Temperature: 350-600° C., Cone Gas Flow: 50-150 l/h, Desolvation Gas Flow: 650-1000 l/h, Mass range: 50 to 900 Da and an Acquity UPLC from Waters Corporation: Binary pump, heated column compartment, diode-array detector and ELSD. Column: Waters UPLC HSS T3, 1.8 μm, 30×2.1 mm, Temp: 60° C., DAD Wavelength range (nm): 210 to 400, Runtime: 1.5 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 1.0 min, 100% B isocratic for 0.2 min, 100-10% B in 0.05 min, 10% B isocratic for 0.05 min.

LC-MS Method B: Spectra were recorded on a Mass Spectrometer from Waters Corporation (SQD, SQDII or QDA Single quadrupole mass spectrometer) equipped with an electrospray source (Polarity: positive and negative ions), Optimized Mass Parameter: Ionisation method: Electrospray (ESI); Polarity: Positive and Negative Polarity Switch; Scan Type: Full Scan; Capillary (kV): 0.8; Cone Voltage (V): 23; Source Temperature (° C.): 120; Desolvation Gas Flow (L/Hr): 1000; Desolvation Temperature (° C.): 600; Gas Flow @ Cone (L/Hr): 50; Mass range: 110 to 1200 Da; Gradient conditions: solvent A: water with 0.1% formic acid: acetonitrile=95:5 v/v; solvent B: acetonitrile with 0.05% formic acid

| Time (minutes) | A (%) | B (%) | Flow rate (ml/min) |
|---|---|---|---|
| 0 9 | 0 | 10 | 0.6 |
| 0.2 | 90 | 10 | 0.6 |
| 0.3 | 50 | 50 | 0.6 |
| 0.6 | 0 | 100 | 0.6 |
| 1.3 | 0 | 100 | 0.6 |
| 1.4 | 90 | 10 | 0.6 |
| 1.6 | 90 | 10 | 0.6 |

PDA Wavelength range: 200 to 400 nm, Column: Acquity UPLC HSS T3 C18; Column length: 30 mm; internal diameter of column: 2.1 mm; Particle Size: 1.8 u; Column oven temperature: 40° C.

LC-MS Method C: Spectra were recorded on a Mass Spectrometer from Waters Corporation (SQD, SQDII or QDA Single quadrupole mass spectrometer) equipped with an electrospray source (Polarity: positive and negative ions), Optimized Mass Parameter: Ionisation method: Electrospray (ESI); Polarity: Positive and Negative Polarity Switch; Scan Type: Full Scan; Capillary (KV): 3.00; Cone Voltage (V): 41.00; Source Temperature (° C.): 150; Desolvation Gas Flow (L/Hr): 1000; Desolvation Temperature (° C.): 500; Gas Flow @ Cone (L/Hr): 50; Mass range: 110 to 1000 Da; Gradient conditions: solvent A: water with 0.1% formic acid: acetonitrile=95:5 v/v; solvent B: acetonitrile with 0.05% formic acid

| Time (minutes) | A (%) | B (%) | Flow rate (ml/min) |
|---|---|---|---|
| 0 | 90 | 10 | 0.6 |
| 0.2 | 90 | 10 | 0.6 |
| 0.3 | 50 | 50 | 0.6 |
| 0.6 | 0 | 100 | 0.6 |
| 1.3 | 0 | 100 | 0.6 |
| 1.4 | 90 | 10 | 0.6 |
| 1.6 | 90 | 10 | 0.6 |

PDA Wavelength range: 200 to 400 nm,
Column: Acquity UPLC HSS T3 C18; Column length: 30 mm; Internal diameter of column: 2.1 mm; Particle Size: 1.8 u; Column oven temperature: 40° C.

LC-MS Method D: Spectra were recorded on a Mass Spectrometer from Waters Corporation (SQD, SQDII or QDA Single quadrupole mass spectrometer) equipped with an electrospray source (Polarity: positive and negative ions), Optimized Mass Parameter: Ionisation method: Electrospray (ESI); Polarity: Positive and Negative Polarity Switch, Scan Type: MS2 Scan; Capillary (kV): 4.00; Fragmentor (V): 100.00; Gas Temperature (° C.): 350; Gas Flow (L/min): 11; Nebulizer Gas (psi): 40; Mass range: 110 to 1000 Da; Detection (VWD): 254 nm; Gradient conditions: solvent A: water with 0.1% formic acid: acetonitrile=95:5 v/v; solvent B: acetonitrile with 0.1% formic acid

| Time (minutes) | A (%) | B (%) | Flow rate (ml/min) |
|---|---|---|---|
| 0 | 90 | 10 | 1.8 |
| 0.9 | 0 | 100 | 1.8 |
| 1.8 | 0 | 100 | 1.8 |
| 2.2 | 90 | 10 | 1.8 |
| 2.5 | 90 | 10 | 1.8 |

Column: KINETEX EVO C18, Column length: 50 mm; Internal diameter of column: 4.6 mm; Particle Size: 2.6 μ, Column oven temperature: 40° C.

LC-MS Method E: Instrument specifications: UHPLC Agilent 1290 Series LCMSD system with DAD\ELSD and Agilent LC\MSD (G⁶¹²⁵B) mass-spectrometer; Column: Agilent Poroshell 120 SB-C18 4.6×30 mm 2.7 μm; Column Temperature: 60° C.; Mobile phase: A-water (0.1% formic acid), B-acetonitrile (0.1% formic acid); Flow rate: 3 ml/min; Gradient: 0.01 min-1% B, 1.5 min-100% B, 1.73 min-100% B; MS Ionization mode: Electrospray ionization (ESI); MS Scan range: 83-600 m/z; UV detection: 215 nm, 254 nm, 280 nm LC-MS Method F: Spectra were recorded on a Mass Spectrometer from Agilent Technologies (6410 Triple Quadrupole mass spectrometer) equipped with an electrospray source (Polarity: positive or negative ions, MS2 Scan, Capillary: 4.00 kV, Fragmentor: 100 V, Desolvation Temperature: 350° C., Gas Flow: 11 L/min, Nebulizer Gas: 45 psi, Mass range: 110 to 1000 Da) and a 1200 Series HPLC from Agilent: quaternary pump, heated column compartment and VWD detector. Column: KINETEX EVO C18, 2.6 μm, 50×4.6 mm, Temp: 40° C., Detector VWD Wavelength: 254 nm, Solvent Gradient: A=water+5% Acetonitrile+0.1% HCOOH, B=Acetonitrile+0.1% HCOOH: gradient: 0 min 10% B, 90% A; 0.9-1.8 min 100% B; 1.8-2.2 min 100-10% B; 2.2-2.5 min 10% B; Flow (mL/min) 1.8.

LC-MS Method G: Spectra were recorded on a Mass Spectrometer from Waters (SQD2 or QDA Single quadrupole mass spectrometer) equipped with an electrospray source (Polarity: Positive and Negative Polarity Switch), Capillary: 0.8-3.00 kV, Cone range: 25 Source Temperature: 120-150° C., Desolvation Temperature: 500-600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0.-0.2 min 10-50% B; 0.2-0.6 min 50-100% B; 0.6-1.3 min 100% B; 1.3-1.4 min 100-10% B; 1.4-1.6 min 10% B; Flow (mL/min) 0.6.

LC-MS method H: Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 KV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 3.0 min; Flow (ml/min) 0.75.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | — | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether (4-5 mol of ethylene oxide) | 3% |
| calcium dodecylbenzene sulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### Abbreviations

CDCl₃ deuterated chloroform
DABCO 1,4-diazabicyclo[2.2.2]octane, also known as triethylenediamine or TEDA
DCC dicyclohexyl carbodiimide
DIPEA Diisopropylethylamine (N,N-Diisopropylethylamine)
DMA dimethylacetamide
DMF dimethylformamide
DMSO dimethyl sulfoxide
DMSO-d6 deuterated Dimethyl sulfoxide
EDC 1-ethyl-3-(3-dimethylaminopropyl) carbodiimide
Et₃N triethylamine
EtOAc ethylacetate
HATU 1-[bis(dimethylamino)methylene]-1H-1,2,3-triazolo[4,5-b]pyridinium 3-oxide hexafluorophosphate
HCl hydrochloric acid
hr/hrs hour/hours
LC-MS Liquid Chromatography Mass Spectrometry (LC-MS, LC/MS or LCMS)
rh relative humidity
rt room temperature
Rt retention time
ssp. subspecies
TBME methyl tert-butyl ether or tert-butyl methyl ether

### Preparatory Examples

The compounds of formula (I) according to the invention may be prepared using the synthetic techniques described both above and below.

“Mp” means melting point in° C. Free radicals represent methyl groups.

### Example P1: Preparation of [2-(2,4-difluorophenyl)tetrazol-5-yl]-[6-methoxy-1-methyl-4-(1-methylpyrazol-4-yl)-3,4-dihydro-1H-isoquinolin-2-yl]methanone (Compound P-31, Table P)

### Step 1: Preparation of 4-(1,5-dimethylpyrazol-4-yl) isoquinoline

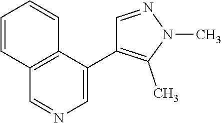 [SMILES: Cc1c(-c2cncc3ccccc23)cnn1C]

In a microwave vial, a suspension of 4-bromoisoquinoline (2.0 g, 9.4204 mmol), 1,5-dimethyl-4-(4,4,5,5-tetramethyl-1,3,2-dioxaborolan-2-yl)-1H-pyrazole (2.3484 g, 10.362 mmol.) and potassium carbonate (1.4322 g, 10.362 mmol) in a toluene/methanol mixture (30 mL, 5:1) was degassed with argon for several minutes, and then tetrakis(triphenylphosphaniumyl)palladium (0.545 g, 0.471 mmol) was added. The reaction mixture was heated at 100° C. and stirred for 1 hour under microwave irradiation. After cooling down to rt, the reaction mixture was poured into water, extracted with EtOAc, the organic layer separated, dried over Na₂SO₄, filtered, and concentrated in vacuo. Purification of the crude material by flash chromatography over silica gel (eluting with EtOAc/30% methanol) afforded the title compound.

LCMS (Method A): m/z 225 [M+H], retention time 0.51 min

### Step 2: Preparation of 4-(1,5-dimethylpyrazol-4-yl)-1,2,3,4-tetrahydroisoquinoline

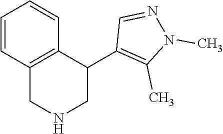 [SMILES: Cc1c(C2CNCc3ccccc32)cnn1C]

To a solution of 4-(1,5-dimethylpyrazol-4-yl) isoquinoline (2.00 g, 8.96 mmol) in methanol (90 mL) was added at rt sodium cyanoborohydride (3.55 g, 53.7 mmol). The reaction mixture was stirred at rt (room temperature), and then hydrochloric acid (1.25 M in methanol) was added until the pH reached 2-3. After 30 minutes of stirring at rt, the reaction mixture was diluted with water and basified with 2 N sodium hydroxide, and the mixture extracted with EtOAc (3×25 mL). The combined organic layers were dried over Na₂SO₄, filtered, and concentrated in vacuo. The resulting yellow oil was used without further purification.

LCMS (Method A): m/z 228 [M+H]. retention time 0.29 min

The hydrochloride salt of the title product (4-(1,5-dimethylpyrazol-4-yl)-1,2,3,4-tetrahydroisoquinoline; hydrochloride) could be obtained by treating the yellow oil with 2 M HCl in diethyl ether and then concentration in vacuo.

### Step 3: Preparation of ethyl(2E)-2-(p-tolylsulfonylhydrazono)acetate

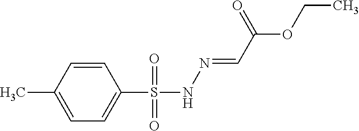 [SMILES: CCOC(=O)/C=N/NS(=O)(=O)c1ccc(C)cc1]

A solution of 4-methylbenzenesulfonohydrazide (2 g, 10.7 mmol) in ethanol (40 mL) was treated with ethyl 2-oxoacetate (2.63 g, 12.8 mmol) at rt. The reaction mixture was stirred for 1 hour and the ethanol removed by concentration in vacuo. The residue obtained was diluted with water and extracted with EtOAc. The organic phase was separated, and the solvent was evaporated in vacuo to afford ethyl(2E)-2-(p-tolylsulfonylhydrazono)acetate which was used as such for next step.

LCMS (Method B): m/z 271 [M+H], retention time 1.06 min.

¹H NMR (400 MHz, CDCl₃) 0 ppm 9.31 (s, 1H), 7.85 (d, J=8.31 Hz, 2H), 7.34 (d, J=8.07 Hz, 2H), 7.23 (s, 1 H), 4.27 (q, J=7.09 Hz, 2H), 2.44 (s, 3H) 1.23-1.37 (m, 3H).

### Step 4: Preparation of ethyl 2-(2,4-difluorophenyl)tetrazole-5-carboxylate

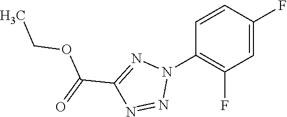 [SMILES: CCOC(=O)c1nnn(-c2ccc(F)cc2F)n1]

A solution of 2,4-difluoroaniline (1.00 g, 7.74 mmol) in a solution of 6M hydrochloric acid in deionized water (6 mL, 36 mmol) and ethanol (5 mL) was cooled to 0° C. To that solution was added sodium nitrite (0.64 g, 9.29 mmol) and the resulting solution stirred for 1 hour, and then added dropwise to a solution of ethyl(2E)-2-(p-tolylsulfonylhydrazono)acetate (2.3 g, 8.51 mmol) in pyridine (20 mL) at −20° C. The reaction mixture was slowly allowed to warm to rt and then stirred for 5 hrs. The resulting mixture was diluted with 1N HCl and the aqueous layer was extracted with EtOAc. The combined organic layers were dried over Na₂SO₄ and concentrated in vacuo to give the crude product which was purified by column chromatography eluting with 30% EtOAc in hexane to give ethyl 2-(2,4-difluorophenyl)tetrazole-5-carboxylate as a reddish oil.

¹H NMR (400 MHz, CDCl₃) 0 ppm 7.86-7.94 (m, 1H), 7.11-7.21 (m, 2H), 4.60 (q, J=7.21 Hz, 2H), 1.51 (t, J=7.13 Hz, 3H)

### Step 5: Preparation of [[2-(2,4-difluorophenyl)tetrazol-5-yl]-[4-(1,5-dimethylpyrazol-4-yl)-3,4-dihydro-1H-isoquinolin-2-yl]methanone (Compound P-31, Table P)

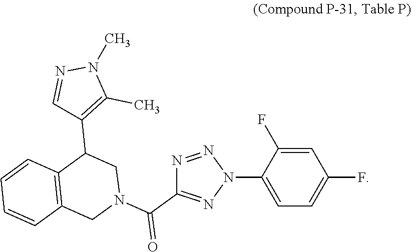 [SMILES: Cc1c(C2CN(C(=O)c3nnn(-c4ccc(F)cc4F)n3)Cc3ccccc32)cnn1C]

A solution of ethyl 2-(2,4-difluorophenyl)tetrazole-5-carboxylate (0.1 g, 0.39 mmol) in toluene (2 mL), was treated with 4-(1,5-dimethylpyrazol-4-yl)-1,2,3,4-tetrahydroisoquinoline-hydrochloride (0.12 g, 0.47 mmol), and the solution then cooled with ice bath. To this was added trimethylaluminum solution (2.0 mol/L) in toluene (0.6 mL, 1.18 mmol) dropwise at 0° C. and the resulting mixture then heated to 80° C. for 2 hrs. After completion of reaction the mixture was diluted with EtOAc and quenched with aqueous 1 N HCl. The organic layer was separated, and the aqueous layer backextracted with EtOAc. The combined organic layers were dried over Na₂SO₄, filtered, and concentrated in vacuo. The crude product was purified by column chromatography to give [2-(2,4-difluorophenyl)tetrazol-5-yl]-[4-(1,5-dimethylpyrazol-4-yl)-3,4-dihydro-1H-isoquinolin-2-yl]methanone as white solid.

LCMS (Method C): m/z 436 (M+H). retention time 1.07 min.

### Example P2: Preparation of [1-(2,4-difluorophenyl)triazol-4-yl]-[4-(1-methylpyrazol-4-yl)-3,4-dihydro-1H-isoquinolin-2-yl]methanone (Compound P-8, table P)

### Step 1: Preparation of methyl 1-(2,4-difluorophenyl)triazole-4-carboxylate

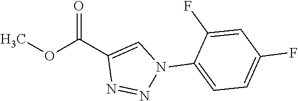 [SMILES: COC(=O)c1cn(-c2ccc(F)cc2F)nn1]

A solution of 1-azido-2,4-difluoro-benzene (0.30 g, 1.8 mmol) in methanol (3.6 mL) was treated successively with copper (II) sulphate anhydrous (0.053 g, 0.33 mmol), sodium ascorbate (0.46 g, 2.3 mmol) in water (3.6 mL), and then methyl prop-2-ynoate (0.14 g, 0.14 mL, 1.7 mmol). The reddish reaction mixture was stirred for 2 days at room temperature monitoring by LCMS. The reaction mixture was then concentrated in vacuo, and the residue taken up in water and EtOAc. The organic layer was separated, washed with brine, and the separated organic layer dried over MgSO₄, filtered, and concentrated in vacuo. The crude product was purified over a silica gel cartridge (Rf200), eluting with cyclohexane/EtOAc, to give the title compound as a white solid.

LCMS (Method A): m/z 240 (M+H), retention time 0.79 min.

¹H NMR (400 MHz, CDCl₃) 0 ppm; 8.59 (d, J=2.57 Hz, 1H), 7.96-8.05 (m, 1H), 7.09-7.17 (m, 2H), 4.02 (s, 3H)

### Step 2: Preparation of 1-(2,4-difluorophenyl)triazole-4-carboxylic acid

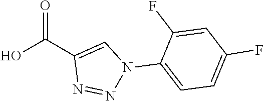 [SMILES: O=C(O)c1cn(-c2ccc(F)cc2F)nn1]

A sample of methyl 1-(2,4-difluorophenyl)triazole-4-carboxylate (0.31 g, 1.3 mmol) was dissolved in tetrahydrofuran (6.5 mL) and water (3.2 mL) under argon to give a pale brown solution. To this was added lithium hydroxide monohydrate (0.047 g, 1.9 mmol), and the mixture was stirred at room temperature. After 3 hrs, LCMS shows desired mass and consumption of starting material. The tetrahydrofuran was evaporated in vacuo, and then aqueous HCl 2N (ca. 0.5 mL) was added to leave the aqueous phase with a pH 3-4. The resulting lightly red suspension was filtered, the white cake washed with water and cyclohexane and dried in vacuo at 55° C. to give 1-(2,4-difluorophenyl)triazole-4-carboxylic acid as white solid.

LCMS (Method A): m/z 226 (M+H), retention time 0.63 min. ¹H NMR (400 MHz, DMSO-d6) o ppm 7.27-7.52 (m, 1H), 7.73 (ddd, J=11.10, 8.71, 2.57 Hz, 1H), 7.95 (td, J=8.80, 5.87 Hz, 1H), 9.15 (d, J=1.47 Hz, 1H), 13.26-13.60 (bs, 1H).

### Step 3: Preparation of 4-(1-methylpyrazol-4-yl) isoquinoline

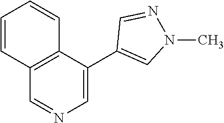 [SMILES: Cn1cc(-c2cncc3ccccc23)cn1]

Prepared analogously to example P1, step 1

LCMS (Method A): m/z 210 (M+H), retention time 0.41 min

¹H NMR (400 MHz, CDCl₃) 0 ppm: 9.20 (s, 1H), 8.51 (s, 1H), 8.10-8.18 (m, 1H), 8.03 (d, J=8.07 Hz, 1H), 7.78-7.81 (m, 1H), 7.70-7.76 (m, 1H), 7.66-7.68 (m, 1H), 7.62-7.66 (m, 1H), 4.06 (s, 3H)

### Step 4: Preparation of 4-(1-methylpyrazol-4-yl)-1,2,3,4-tetrahydroisoquinoline

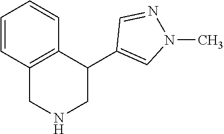 [SMILES: Cn1cc(C2CNCc3ccccc32)cn1]

Prepared analogously to example P1, step 2. The crude product was used without further purification.

### Step 5: Preparation of [1-(2,4-difluorophenyl)triazol-4-yl]-[4-(1-methylpyrazol-4-yl)-3,4-dihydro-1H-isoquinolin-2-yl]methanone (compound P-8, Table P)

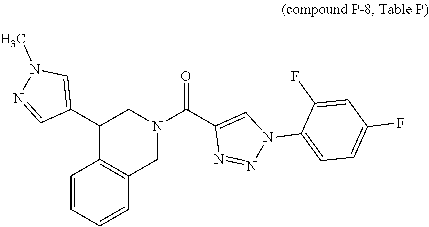 [SMILES: Cn1cc(C2CN(C(=O)c3cn(-c4ccc(F)cc4F)nn3)Cc3ccccc32)cn1]

Under argon, a mixture of 4-(1-methylpyrazol-4-yl)-1,2,3,4-tetrahydroisoquinoline (0.090 g, 0.40 mmol) and 1-(2,4-difluorophenyl)triazole-4-carboxylic acid (0.090 g, 0.40 mmol), dissolved in EtOAc (3 mL), was treated with 1-propanephosphonic anhydride (0.60 mL, 1.0 mmol) and N,N-Diisopropylethylamine (0.28 mL, 1.6 mmol). The resulting solution was stirred at room temperature. After 17 hrs, LCMS showed reaction completion. The reaction mixture was poured in water and extracted with EtOAc. The organic layer was washed with saturated sodium bicarbonate solution, followed by 1M HCl, water and brine. The organic layer was dried over Na₂SO₄ and concentrated in vacuo. Purification of the crude material by flash chromatography over silica gel (eluting with EtOAc in cyclohexane) afforded the title product.

LCMS (Method A) m/z 421 (M+H), retention time 0.92 min.

### Example P3: Preparation of [2-(2,4-difluorophenyl)tetrazol-5-yl]-[4-methyl-4-(1-methylpyrazol-4-yl)-1,3-dihydroisoquinolin-2-yl]methanone (Compound P-32, Table P)

### Step 1: Preparation of (1-methylpyrazol-4-yl)-phenyl-methanol

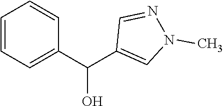

A one necked round bottom flask, equipped with a magnetic stirrer bar, was charged with 1-methyl-1H-pyrazole-4-carbaldehyde (2.20 g, 19.2 mmol) and tetrahydrofuran (40 mL). To the colorless solution, phenylmagnesium bromide 1 molar in THF (21 mL, 21.1 mmol) was added dropwise at 0-5° C. under argon atmosphere for 15 minutes. After the addition, the ice-bath was removed, and the white suspension was stirred at room temperature for 2.5 hrs. The reaction mixture was poured into saturated ammonium chloride solution (40 mL) and extracted with EtOAc (2×40 mL). The combined organic layers were washed with brine, dried over Na₂SO₄, and concentrated in vacuo to afford the crude product as a colorless oil. The crude product was purified by combi flash (silica gel, gradient: EtOAc in cyclohexane) to afford the desired product (1-methylpyrazol-4-yl)-phenyl-methanol as a colorless oil.

LCMS (Method A) m/z 189 [M+H], retention time: 0.62 min. ¹H NMR (400 MHz, CDCl₃) 0 ppm 7.26-7.43 (m, 6H), 7.18 (s, 1H), 5.80 (s, 1H), 3.81 (s, 3H), 2.89 (br s, 1 H)

### Step 2: Preparation of 2-(1-methylpyrazol-4-yl)-2-phenyl-acetonitrile

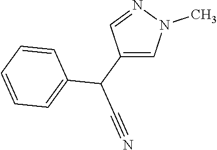

A round-bottom flask, equipped with a magnetic stirrer bar and condenser, was charged with (1-methylpyrazol-4-yl)-phenyl-methanol (3.45 g, 15.6 mmol) and dichloromethane (156 mL). Then, lithium carbonate (0.23 g, 3.1 mmol), trimethylsilyl cyanide (9.0 mL) and lodine (7.23 g, 28.0 mmol) were added successively at room temperature. The mixture was stirred at 35° C. for 1 hr. The reaction mixture was then cooled to room temperature and poured into saturated sodium thiosulphate (250 mL) and extracted with dichloromethane (2×150 mL). The combined organic layers were washed with brine, dried over Na₂SO₄, and concentrated in vacuo to afford the crude product which was purified by combi flash (silica gel, gradient: EtOAc in cyclohexane) to afford desired title compound as a yellow oil.

LCMS (method A) m/z 198 [M+H], retention time: 0.78 min.

¹H NMR (400 MHz, CDCl₃) 0 ppm 7.33-7.43 (m, 6H), 7.32 (s, 1H), 5.09 (s, 1H), 3.87 (s, 3H)

### Step 3: Preparation of 2-(1-methylpyrazol-4-yl)-2-phenyl-propanenitrile

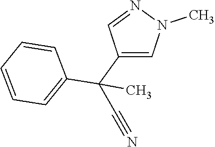 [SMILES: Cn1cc(C(C)(C#N)c2ccccc2)cn1]

A 250 mL-3-necked flask, equipped with a magnetic stirrer bar and condenser, was charged with 2-(1-methylpyrazol-4-yl)-2-phenyl-acetonitrile (3.22 g, 16.3 mmol) and tetrahydrofuran (65 mL). A solution of n-butyllithium in hexane (7.8 mL, 19.6 mmol) was added dropwise at .-70° C. under a nitrogen atmosphere (small exothermic to −65° C. was observed). The orange solution was stirred at this temperature for 30 minutes before adding iodomethane (1.54 mL, 24.5 mmol) dropwise at −70° C. The resulting yellow solution was stirred at −78° C. for 5 minutes and then allowed to warm to ambient temperature and stirred for 30 minutes. The reaction mixture was then poured into water (90 mL) and extracted with EtOAc. The organic layer was washed with brine, dried over Na₂SO₄, and concentrated in vacuo to afford the crude product as an orange oil. This was purified by combi flash (silica gel, gradient: EtOAc in cyclohexane) to afford the title compound as a yellow oil.

LCMS (Method A) m/z 211 [M+H], retention time: 0.84 min.

¹H NMR (400 MHz, CDCl₃) 0 ppm 7.28-7.49 (m, 7H), 3.88 (s, 3H), 2.04 (s, 3H)

### Step 4: Preparation of 2-(1-methylpyrazol-4-yl)-2-phenyl-propan-1-amine

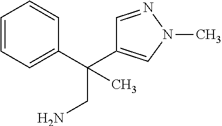 [SMILES: Cn1cc(C(C)(CN)c2ccccc2)cn1]

A 250 mL-3-necked flask, equipped with a magnetic stirrer bar, was charged with 2-(1-methylpyrazol-4-yl)-2-phenyl-propanenitrile (2.82 g, 13.3 mmol) and tetrahydrofuran (40 mL). To the yellow solution borane dimethyl sulfide complex (4.0 mL, 40.0 mmol) was added dropwise at room temperature under argon atmosphere and the resulting colorless mixture was stirred at 65° C. for 2 hrs. The reaction mixture was cooled to 0° C. before adding hydrochloric acid (8.9 mL, 53.7 mmol) dropwise (strong gas evolution) and the mixture was stirred at 65° C. for 1 hr and allowed to stand overnight at rt. The mixture was diluted with water (80 mL), basified with 13 mL NaOH 6M (pH 12) and the mixture extracted twice with EtOAc. The combined organic layers were washed with brine, dried over Na₂SO₄, and concentrated in vacuo to give the title compound as a yellow oil which was used without further purification in the next step.

LCMS (Method A) m/z 216 [M+H], retention time: 0.39 min

### Step 5: Preparation of methyl N-[2-(1-methylpyrazol-4-yl)-2-phenyl-propyl]carbamate

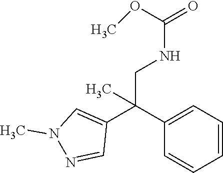 [SMILES: COC(=O)NCC(C)(c1ccccc1)c1cnn(C)c1]

A sealed tube, equipped with a magnetic stirrer bar, was charged with 2-(1-methylpyrazol-4-yl)-2-phenyl-propan-1-amine (3.01 g, 11.2 mmol) and dichloromethane (45 mL). Methyl chloroformate (1.1 mL, 13.4 mmol), followed by triethylamine (4.7 mL, 33.6 mmol) were added dropwise at 0-10° C. under an argon atmosphere. The ice-bath was removed, and the mixture was stirred at room temperature for 1 h. The reaction mixture was poured into water and the organic phase was separated. The water phase was extracted twice with EtOAc. The combined organic layers were dried over Na₂SO₄ and concentrated in vacuo. The crude material was purified by combiflash (silica gel, gradient: EtOAc in cyclohexane) to afford the title compound as a colorless gum.

LCMS (method A) m/z 274 [M+H], retention time: 0.80 min.

### Step 6: Preparation of methyl 4-methyl-4-(1-methylpyrazol-4-yl)-1,3-dihydroisoquinoline-2-carboxylate

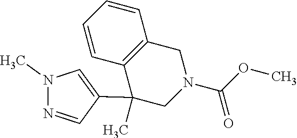

A one necked round bottom flask, equipped with a magnetic stirrer bar, was charged with methyl N-[2-(1-methylpyrazol-4-yl)-2-phenyl-propyl]carbamate (422 mg, 1.54 mmol), hydrochloric acid (5.00 mL/mmol, 9.26 g, 7.72 mL, 94.0 mmol) and paraformaldehyde (93 mg, 0.97 mmol). The mixture was stirred at rt for 40 min, where LCMS analysis showed reaction completion. The reaction mixture was slowly poured into water (30 mL) neutralized with sodium bicarbonate and extracted with EtOAc (3×20 mL). The combined organic layers were washed with brine, dried over Na₂SO₄, and concentrated in vacuo to yield the crude title compound as a colorless gum which was used as such without further purification.

LCMS (method A) m/z 286 [M+H], retention time: 0.87 min.

### Step 7: Preparation of 4-methyl-4-(1-methylpyrazol-4-yl)-2,3-dihydro-1H-isoquinoline

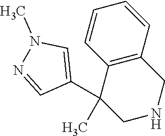 [SMILES: Cn1cc(C2(C)CNCc3ccccc32)cn1]

A one necked round bottom flask, equipped with a magnetic stirrer bar, was charged methyl 4-methyl-4-(1-methylpyrazol-4-yl)-1,3-dihydroisoquinoline-2-carboxylate (3.86 g, 13.5 mmol), 1,2-dichloroethane (5.00 ml/mmol, 68 mL) and iodotrimethylsilane (8.37 g, 5.69 mL, 40.6 mmol). The mixture was stirred at 60° C. under argon atmosphere for 45 min (LCMS analysis showed reaction completion). After cooling to rt, the reaction mixture was slowly poured into saturated sodium bicarbonate solution (30 mL). The resulting mixture was extracted with EtOAc (2×50 ml), and the combined organic layers were washed with brine, dried over Na₂SO₄, and concentrated in vacuo to yield the title compound as a dark orange gum.

LCMS (method A), m/z 228 [M+H], retention time: 0.61 min.

### Step 8: Preparation of [2-(2,4-difluorophenyl)tetrazol-5-yl]-[4-methyl-4-(1-methylpyrazol-4-yl)-1,3-dihydroisoquinolin-2-yl]methanone (Compound P-32, Table P)

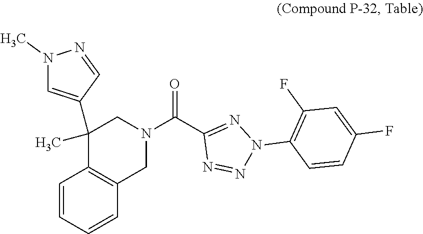 [SMILES: Cn1cc(C2(C)CN(C(=O)c3nnn(-c4ccc(F)cc4F)n3)Cc3ccccc32)cn1]

To a cooled solution of ethyl 2-(2,4-difluorophenyl)tetrazole-5-carboxylate (0.2 g, 0.78 mmol) and 4-methyl-4-(1-methylpyrazol-4-yl)-2,3-dihydro-1H-isoquinoline (0.21 g, 0.94 mmol in toluene (48 mL) was added trimethylaluminum solution (2.0 mol/L) in toluene (1.2 mL, 2.36 mmol) dropwise at 0° C. The resulting mixture was heated to 90° C. for 3 hrs, where LCMS analysis showed reaction completion. The reaction mixture was diluted with EtOAc and quenched with 1 N HCl solution. The mixture was extracted with EtOAc (3×25 mL), and the combined organic layer dried over Na₂SO₄, filtered, and concentrated in vacuo. The crude product was purified by chromatography over silica gel with a gradient cyclohexane/EtOAc to afford [2-(2,4-difluorophenyl)tetrazol-5-yl]-[4-methyl-4-(1-methylpyrazol-4-yl)-1,3-dihydroisoquinolin-2-yl]methanone.

LCMS (Method C); m/z 436 [M+H], retention time: 1.06 min.

¹H NMR (400 MHz, CDCl₃) 0 ppm 7.79-7.88 (m, 1H), 6.99-7.22 (m, 7H), 6.72 (s, 1H), 5.10-5.21 (m, 1 H), 4.87-5.00 (m, 1H), 4.23 (d, J=13.45 Hz, 1H), 3.84-3.96 (m, 1H), 3.67 (s, 3H), 1.59 (s, 3H)

### Example P4: Preparation of [1-(2,4-difluorophenyl)-1,2,4-triazol-3-yl]-[4-(1,5-dimethylpyrazol-4-yl)-3,4-dihydro-1H-isoquinolin-2-yl]methanone (Compound P-33, Table P)

### Step 1: Preparation of methyl 1-(2,4-difluorophenyl)-1,2,4-triazole-3-carboxylate

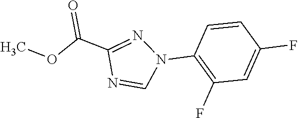 [SMILES: COC(=O)c1ncn(-c2ccc(F)cc2F)n1]

Prepared analogously to that described in Org. Lett. 2018, 20, 6930-6933. Thus, a solution of 2,4-difluorobenzenediazonium-tetrafluoroborate (prepared as described in J. Am. Chem. Soc. 1956, 78, 2593-6, 500 mg, 0.5 g, 2.1939 mmol) in tetrahydrofuran (8.8 mL) was cooled to 0° C. and treated with lithium acetate dihydrate (0.44765 g, 4.3879 mmol), Cu(OAc)₂ (0.10 equiv., 0.03985 g, 0.21939 mmol) and methyl 2-isocyanoacetate (1.20 equiv., 0.2609 g, 0.239 mL, 2.6327 mmol) were added at 0° C. and the mixture was stirred at this temperature for 4h. LCMS analysis showed reaction completion after this time. The reaction mixture was poured into water (25 mL) and the mixture was extracted with EtOAc (2×20 mL). The combined organic layers were washed with brine, dried over Na₂SO₄, and concentrated in vacuo. The crude material was purified by column chromatography (24 g SiO₂, eluting with an EtOAc/Cyclohexane gradient) to give the title compound as an oil.

¹H NMR (400 MHz, CDCl₃) 0 ppm 8.68 (d, J=2.81 Hz, 1H), 7.93-8.00 (m, 1H), 7.08-7.14 (m, 2H), 4.08 (s, 3H)

### Step 2: Preparation of [1-(2,4-difluorophenyl)-1,2,4-triazol-3-yl]-[4-(1,5-dimethylpyrazol-4-yl)-3,4-dihydro-1H-isoquinolin-2-yl]methanone (Compound P-33, Table P)

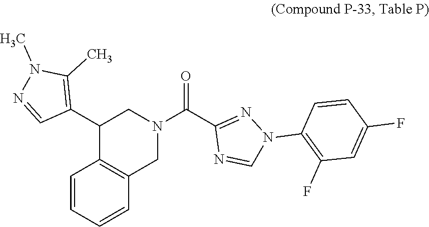 [SMILES: Cc1c(C2CN(C(=O)c3ncn(-c4ccc(F)cc4F)n3)Cc3ccccc32)cnn1C]

A sample of 4-(1,5-dimethylpyrazol-4-yl)-1,2,3,4-tetrahydroisoquinoline hydrochloride (0.13 g, 0.50 mmol, prepared as described in Example P1) was added to a solution methyl 1-(2,4-difluorophenyl)-1,2,4-triazole-3-carboxylate (0.100 g, 0.41 mmol) in toluene (3.0 mL) in microwave vial. This reaction mixture was stirred under argon atmosphere for 5 minute and then bis(trimethylaluminum)-1,4-diazabicyclo[2.2.2]octane adduct (0.27 g, 1.04 mmol) was added. The reaction mixture was stirred at rt for 5 minutes then stirred for 2 hrs at 70° C. in the microwave. After cooling to rt, the reaction mixture was diluted with water, and extracted with EtOAc (X³). The combined organic layers were washed with brine, dried over Na₂SO₄, and concentrated in vacuo. The crude product was purified by chromatography on silica gel, using a 50-70% cyclohexane/EtOAc as eluent system, to afford [1-(2,4-difluorophenyl)-1,2,4-triazol-3-yl]-[4-(1,5-dimethylpyrazol-4-yl)-3,4-dihydro-1H-isoquinolin-2-yl]methanone as gummy mass.

LCMS (Method D): m/z (M+H)₄₃₅, retention time 1.38 min

¹H NMR (400 MHz, CDCl₃) 0 ppm 8.59-8.50 (m, 1H), 7.89-7.75 (m, 1H), 7.25-6.92 (m, 7H), 5.24-5.13 (m, 1H) 4.78 (d, 1H) 4.15-4.49 (m, 2H), 3.70-3.52 (m, 4H), 2.18-1.98 (m, 3H)

### Example P5: Preparation of [2-(2,4-difluorophenyl)triazol-4-yl]-[rac-(1S,4S)-4-(1,5-dimethylpyrazol-4-yl)-1-methyl-3,4-dihydro-1H-isoquinolin-2-yl]methanone (Compound P-40, Table P)

### Step 1:(1,5-dimethylpyrazol-4-yl)-phenyl-methanol

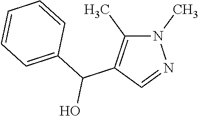

Preparation as in example 3, step 1

LCMS (Method A): m/z (M+H)₂₀₃, retention time 0.68 min

¹H NMR (400 MHz, CDCl₃) 0 ppm 2.23 (s, 3H)_{2.28} (d, J=4.00 Hz, 1H) 3.76 (s, 3H) 5.80 (d, J=3.63 Hz, 1H) 7.22 (s, 1H) 7.27-7.32 (m, 1H) 7.33-7.44 (m, 4H)

### Step 2:2-(1,5-dimethylpyrazol-4-yl)-2-phenyl-acetonitrile

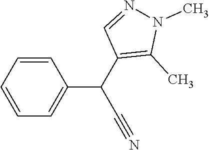 [SMILES: Cc1c(C(C#N)c2ccccc2)cnn1C]

Preparation as in Example 3, step 2

LCMS (Method A): m/z (M+H)₂₁₂, retention time 0.82 min

¹H NMR (400 MHz, CDCl₃) δ=7.46-7.28 (m, 6H), 5.05 (s, 1H), 3.78 (s, 3H), 2.18 (s, 3H)

### Step 3:2-(1,5-dimethylpyrazol-4-yl)-2-phenyl-ethanamine

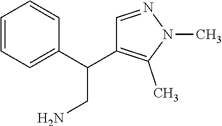 [SMILES: Cc1c(C(CN)c2ccccc2)cnn1C]

Preparation as in Example 3, step 3

LCMS (Method A): m/z (M+H)₂₁₆, retention time 0.60 min

¹H NMR (400 MHz, CDCl₃) 0 ppm 2.09 (s, 3H) 3.20 (dd, J=7.45, 2.00 Hz, 2H) 3.75 (s, 3H) 3.79 (t, J=7.27 Hz, 1H) 7.16-7.25 (m, 3H) 7.26-7.33 (m, 2H) 7.42 (s, 1H)

### Step 4: methyl N-[2-(1,5-dimethylpyrazol-4-yl)-2-phenyl-ethyl]carbamate

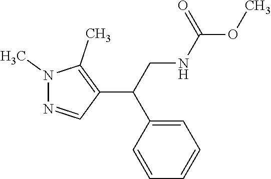

Preparation as in Example 3, step 4

LCMS (Method A): m/z (M+H)₂₇₄, retention time 0.76 min

¹H NMR (400 MHz, CDCl₃) 0 ppm 1.57 (d, J=6.90 Hz, 3H)_{2.18} (br d, J=6.90 Hz, 3H) 3.05-3.27 (m, 1H) 3.78 (br s, 3H) 3.84 (s, 3H) 3.99-4.09 (m, 1H) 4.09-4.38 (m, 1H) 5.22-5.47 (m, 1H) 6.92-7.01 (m, 1H) 7.07-7.27 (m, 4 H ¹H NMR (400 MHz, CDCl₃) o ppm 2.03-2.13 (m, 3H) 3.62-3.74 (m, 5H) 3.77 (s, 3H) 3.94-4.05 (m, 1H) 4.72 (br s, 1H) 7.21-7.26 (m, 3H) 7.27-7.34 (m, 2H) 7.38 (s, 1H)

### Step 5: Preparation of methyl rac-(1S,4S)-4-(1,5-dimethylpyrazol-4-yl)-1-methyl-3,4-dihydro-1H-isoquinoline-2-carboxylate

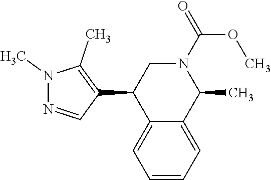

A one necked round bottom flask, equipped with a magnetic stirrer bar, was charged with methyl N-[2-(1,5-dimethylpyrazol-4-yl)-2-phenyl-ethyl]carbamate (2.0 g, 7.3 mmol), hydrochloric acid (37 mL, 450 mmol) and acetaldehyde (0.83 mL, 15 mmol). The mixture was stirred at rt for 2 h. The reaction mixture was slowly poured into water (500 mL) neutralized with NaHCO₃(strong gas evolution). The mixture was extracted with EtOAc (3×50 mL) and the combined organic layers were washed with brine, dried over Na₂SO₄, filtered, and concentrated in vacuo. The crude product was purified by chromatography to afford the title compound as an oil.

LC-MS (Method A): retention time 0.87 min, 300 (M+H)

¹H NMR (400 MHz, CDCl₃) 0 ppm 1.57 (d, J=6.90 Hz, 3H)_{2.18} (br d, J=6.90 Hz, 3H) 3.05-3.27 (m, 1H) 3.78 (br s, 3H) 3.84 (s, 3H) 3.99-4.09 (m, 1H) 4.09-4.38 (m, 1H) 5.22-5.47 (m, 1H) 6.92-7.01 (m, 1H) 7.07-7.27 (m, 4 H

### Step 6. Preparation rac-(1S,4S)-4-(1,5-dimethylpyrazol-4-yl)-1-methyl-1,2,3,4-tetrahydroisoquinoline

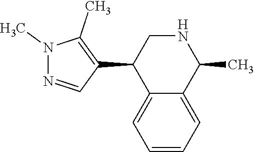

A sample of methyl rac-(1S,4S)-4-(1,5-dimethylpyrazol-4-yl)-1-methyl-3,4-dihydro-1H-isoquinoline-2-carboxylate (1.3 g, 4.1 mmol) in 1,2-dichloroethane (21 mL) was treated with iodotrimethylsilane (1.7 mL, 12 mmol) and the mixture was stirred at 60° C. for 1 h under argon at which time LCMS analysis showed reaction completion. The reaction was cooled to rt and then 10% HCl (22 mL) was added to the reaction under ice cooling. This mixture was concentrated in vacuo to remove the volatiles, and the residual aqueous adjusted pH 8 with 10% NaOH (aq). The mixture was extracted with CH₂Cl₂ (4×25 mL) and the combined organic layers, dried Mg₂SO₄, filtered and concentrated in vacuo to give title product as title compound as a single diastereomer (>97:3 syn:anti) which was pure enough to use for following steps.

LC-MS (Method A): m/z 242 (M+H), retention time 0.35 min

¹H NMR (600 MHz, CDCl₃) o ppm 1.86 (d, J=6.9 Hz, 3H)_{2.23} (s, 3H) 3.27 (dd, J=12.5, 10.8 Hz, 1H) 3.59 (dd, J=12.8, 5.6 Hz, 1H) 3.83 (s, 3H) 4.58 (dd, J=10.6, 5.4 Hz, 1H) 4.80 (q, J=6.8 Hz, 1H) 7.00 (d, J=7.8 Hz, 1H) 7.11 (s, 1H) 7.16 (d, J=7.6 Hz, 1H) 7.18-7.22 (m, 1H) 7.25-7.28 (m, 1H)

### Step 7: Preparation of ethyl 2-[(2,4-difluorophenyl) hydrazono]-3-oxo-propanoate

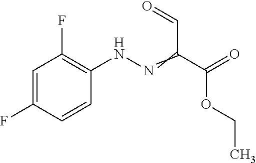 [SMILES: CCOC(=O)C(C=O)=NNc1ccc(F)cc1F]

To a two neck round bottom flask, equipped with a nitrogen inlet, was added 2,4-difluoroaniline (4.00 g, 30.9 mmol) in water (32 mL) and conc. HCl (12 mL, 35%) at rt. The reaction mixture was cooled to 0° C. and to this solution was added a cold solution of sodium nitrite (2.56 g, 37.1 mmol) in water (32 mL) and the resulting mixture stirred it at 0° C. for 5 min. In another round bottom flask, a solution of ethyl 3-(dimethylamino) prop-2-enoate (12.0 g, 23.4 mmol) and potassium acetate (4.61 g, 46.4 mmol) in ethanol (40 mL) was stirred and cooled to 0° C. To this was added the above diazotized solution at 0° C. dropwise, and the reaction mixture allowed to warm to rt and stirred for 18 hrs. LCMS showed formation of the desired product. The reaction was diluted with water and extracted with EtOAc (3×50 mL). The combined organic layers were washed with brine, dried over Na₂SO₄, and concentrated in vacuo to give crude ethyl 2-[(2,4-difluorophenyl) hydrazono]-3-oxo-propanoate as brown solid which was used as such in next step.

LCMS (Method B): m/z 257 [M+H], retention time=1.09 min

### Step 8: Preparation of ethyl 2-[(2,4-difluorophenyl) hydrazono]-3-hydroxyimino-propanoate

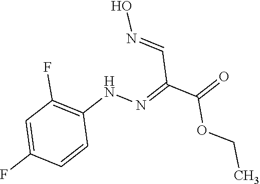 [SMILES: CCOC(=O)C(/C=N/O)=N/Nc1ccc(F)cc1F]

To a solution of ethyl 2-[(2,4-difluorophenyl) hydrazono]-3-oxo-propanoate (3.00 g, 5.85 mmol) in ethanol (30 mL) was added potassium acetate (1.45 g, 14.6 mmol) and hydroxylaminehydrochloride (0.49 g, 7.02 mmol). The reaction mixture was stirred for 2 hrs at 80° C. LCMS showed complete conversion to the desired product. The reaction mixture was cooled to rt and diluted with water and extracted EtOAc (3×50 mL). The combined organic layers were washed with brine, dried over Na₂SO₄, and concentrated in vacuo to give the title compound as a brown solid which was used as such for the next step.

LCMS (Method B): m/z 272 [M+H], retention time=1.16 min

### Step 9: Preparation of ethyl 2-(2,4-difluorophenyl)triazole-4-carboxylate

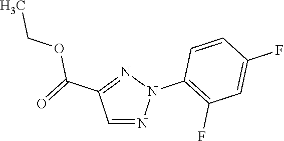 [SMILES: CCOC(=O)c1cnn(-c2ccc(F)cc2F)n1]

A sample of ethyl 2-[(2,4-difluorophenyl) hydrazono]-3-hydroxyimino-propanoate (3.4 g, 6.3 mmol) was treated with acetic anhydride (34 mL, 10 mL/g) at rt. The reaction mixture was then stirred at 140° C. for 1 h. The reaction mass was monitored by LC-MS after completion the reaction mixture was cooled and quenched with ice and cold brine. The reaction mixture was extracted with EtOAc (3×25 mL) and the combined organic layers dried over Na₂SO₄, and concentrated in vacuo, to yield the crude product. This was purified by combiflash using EtOAc/Cyclohexane as eluent (5:95) to afford the title compound as a brown solid.

LCMS (Method C): m/z 254 [M+H], retention time=1.19 min

### Step 10: Preparation of lithium;2-(2,4-difluorophenyl)triazole-4-carboxylate

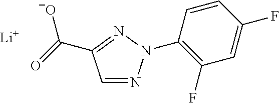 [SMILES: [Li+] ; O=C([O-])c1cnn(-c2ccc(F)cc2F)n1]

To a stirred solution of ethyl 2-(2,4-difluorophenyl)triazole-4-carboxylate (0.10 g, 0.31 mmol) in tetrahydrofuran (0.4 mL) and water (0.1 mL) was treated with lithium hydroxide (11.0 mg 0.47 mmol) and the reaction stirred at for rt for 3 hrs. After completion of reaction, reaction mixture was concentrated in vacuo, and the residue triturated with methyl tert-butyl methyl ether (TBME). This yielded lithium;2-(2,4-difluorophenyl)triazole-4-carboxylate which was used as such for the next step.

LCMS (Method B): m/z 224 [M+H], retention time=0.29 min

### Step 11: Preparation of [2-(2,4-difluorophenyl)triazol-4-yl]-[rac-(1S,4S)-4-(1,5-dimethylpyrazol-4-yl)-1-methyl-3,4-dihydro-1H-isoquinolin-2-yl]methanone (Compound P-40, Table P)

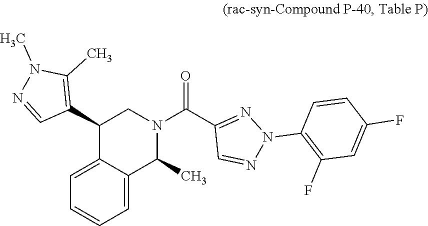 [SMILES: Cc1c([C@H]2CN(C(=O)c3cnn(-c4ccc(F)cc4F)n3)[C@@H](C)c3ccccc32)cnn1C]

A suspension of lithium;2-(2,4-difluorophenyl)triazole-4-carboxylate (0.231 g, 1.0 mmol, 100 mass %) and rac-(1S,4S)-4-(1,5-dimethylpyrazol-4-yl)-1-methyl-1,2,3,4-tetrahydroisoquinoline (0.241 g, 1.0 mmol) was taken in in EtOAc (9 mL) was treated with DIPEA (0.7 g, 5 mmol) and 1-propanephosphonic anhydride (TP3, 2 mL, 3 mmol, 50 mass % in EtOAc) at 0° C. and the reaction mixture was stirred at rt for 2 h. After the completion of the reaction, the reaction mixture was diluted with water (15 mL) and extracted with EtOAc (3×20 mL). The combined organic layers were washed with brine (20 mL), dried over Na₂SO₄, filtered and concentrated in vacuo to give the crude product. This was purified by combiflash by using 0-80% cyclohexane/EtOAc in as an eluent to obtain the title compound as a brown solid.

LCMS (Method B), m/z 449 (M+H), retention time 1.14 min.

### Example P6: Preparation of [2-(2,4-difluorophenyl)tetrazol-5-yl]-[6-methoxy-1-methyl-4-(1-methylpyrazol-4-yl)-3,4-dihydro-1H-isoquinolin-2-yl]methanone (Compound P-34, Table P)

### Step 1: Preparation of (3-methoxyphenyl)-(1-methylpyrazol-4-yl)methanol

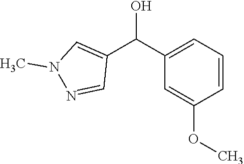

A one necked round bottom flask, equipped with a magnetic stirred bar, was charged with 4-iodo-1-methyl-1 h-pyrazole (5.14 g, 23.5 mmol) and tetrahydrofuran (64 mL). To this solution was added isopropylmagnesium chloride lithium chloride complex (1.3 mol/L) in THF (25 mL, 32.0 mmol) dropwise at 0° C. under argon. The mixture was stirred at 0° C. for 45 min and then 3-methoxybenzaldehyde (2.68 mL, 21.3 mmol) was added dropwise at 0° C. under argon. The mixture was stirred at this temperature for 10 min and then allowed to reach rt and stirred for 2 hrs. The reaction mixture was then poured into water (100 mL) and extracted with EtOAc (2×80 mL). The combined organic layers were washed with brine, dried over Na₂SO₄, and concentrated in vacuo. The crude material was purified by flash chromatography (80 g SiO₂, EtOAc/Cyclohexane gradient) to afford (3-methoxyphenyl)-(1-methylpyrazol-4-yl)methanol.

LCMS (Method B): m/z 219 [M+H], retention time 0.21 min.

¹H NMR (400 MHz, CDCl₃) o ppm: 7.39 (s, 1H), 7.26-7.33 (m, 1H), 7.28 (s, 1H), 6.96-7.05 (m, 2H), 6.82-6.90 (m, 1H), 5.72-5.88 (m, 1H), 3.84 (s, 3H), 3.82 (s, 3H)

### Step 2: Preparation of 2-(3-methoxyphenyl)-2-(1-methylpyrazol-4-yl) acetonitrile

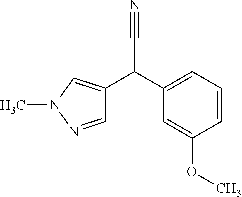

A solution of (3-methoxyphenyl)-(1-methylpyrazol-4-yl)methanol (4 g, 9.16 mmol) in acetonitrile (18 mL) was treated with iodine (4.18 g, 16.5 mmol) and lithium carbonate (0.13 g, 1.83 mmol), and then trimethylsilyl cyanide (5.26 mL, 41.2 mmol) was added dropwise at rt. The resulting mixture was stirred for 8 hrs at 50° C. The reaction mixture was cooled to rt and poured into saturated sodium thiosulphate (400 mL) and extracted with EtOAc (2×100 mL). The combined organic layers were washed with brine, dried over Na₂SO₄, and concentrated in vacuo. The crude product was purified by chromatography over silica gel to afford 2-(3-methoxyphenyl)-2-(1-methylpyrazol-4-yl) acetonitrile.

LCMS (Method B): m/z 228 [M+H], retention time 1.00 min.

¹H NMR (400 MHz, CDCl₃) 0 ppm 7.41 (s, 1H), 7.27-7.37 (m, 2H), 6.87-6.99 (m, 3H), 5.06 (s, 1H), 3.89 (s, 3H), 3.83 (s, 3H)

### Step 3: Preparation of 2-(3-methoxyphenyl)-2-(1-methylpyrazol-4-yl) ethanamine

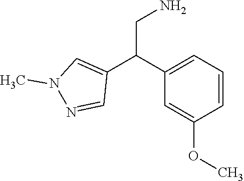

A solution of 2-(3-methoxyphenyl)-2-(1-methylpyrazol-4-yl) acetonitrile (3.2 g, 14 mmol) in tetrahydrofuran (42 mL) was treated dropwise with borane dimethyl sulfide complex (4.0 mL, 42 mmol) at rt under argon. The resulting yellow solution was stirred for 3 hrs at 65° C. and then cooled to 0° C. before adding hydrochloric acid 6 M (9.4 mL, 4.0, 57 mmol) dropwise (strong gas evolution!). The resulting mixture was then stirred for 1 hr at 50° C. and then allowed to cool to rt. The reaction mixture was diluted with water, and then basified with NaOH 6N to pH 12. The mixture was extracted three times with EtOAc, and the combined organic layers were washed once with brine, dried over anhydrous Na₂SO₄ and concentrated in vacuo at to give 2-(3-methoxyphenyl)-2-(1-methylpyrazol-4-yl) ethanamine as a brown oil that was as such for further chemistry.

LCMS (Method B): m/z 232 [M+H], retention time 0.36 min.

¹H NMR (400 MHz, DMSO-d6) o ppm 7.52 (s, 1H), 7.29 (s, 1H), 7.20 (t, J=7.44 Hz, 1H), 6.72-6.87 (m, 3 H), 3.77 (s, 3H), 3.80 (m, 1H), 3.73 (s, 3H), 3.42 (s, 1H) 3.03-2.99 (m, 2H).

### Step 4: Preparation of 6-methoxy-1-methyl-4-(1-methylpyrazol-4-yl)-1,2,3,4-tetrahydroisoquinoline

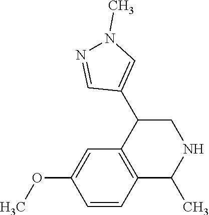

A 25 mL round bottom flask, equipped with a magnetic stirrer bar, was charged with 2-(3-methoxyphenyl)-2-(1-methylpyrazol-4-yl) ethanamine (80 mg, 0.34 mmol), hydrochloric acid 37% (1.7 mL) and acetaldehyde (0.19 mL, 3.46 mmol). The mixture was stirred at rt for 3 hrs and then slowly poured into water and neutralized with saturated aqueous NaHCO₃(strong gas evolution) until pH 8. The mixture was extracted with EtOAc and the combined organic layers washed with brine, dried over Na₂SO₄, and concentrated in vacuo to give the title compound

LCMS (Method B): m/z 258 [M+H], retention time 0.19 min.

### Step 5: Preparation of [2-(2,4-difluorophenyl)tetrazol-5-yl]-[6-methoxy-1-methyl-4-(1-methylpyrazol-4-yl)-3,4-dihydro-1H-isoquinolin-2-yl]methanone (Compound P-34, Table P)

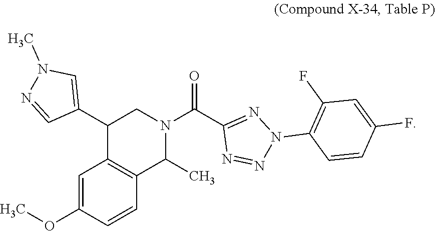 [SMILES: COc1ccc2c(c1)C(c1cnn(C)c1)CN(C(=O)c1nnn(-c3ccc(F)cc3F)n1)C2C]

A mixture of 6-methoxy-1-methyl-4-(1-methylpyrazol-4-yl)-1,2,3,4-tetrahydroisoquinoline (0.25 g, 0.97 mmol), 2-(2,4-difluorophenyl)tetrazole-5-carboxylic acid (0.24 g, 1.06 mmol), N,N-Diisopropylethylamine (0.39 g, 2.91 mmol) and T3P (50 mass %, in EtOAc (1.72 mL, 2.91 mmol)) was stirred in EtOAc (5 mL) at rt overnight. The reaction mixture was diluted with water and EtOAc, and the organic layer removed. The aqueous layer was back-extracted 3 times with EtOAc, and the combined organic layers dried over Na₂SO₄, filtered, and concentrated in vacuo to give the crude product. This was purified by jchromatography over silica gel to give [2-(2,4-difluorophenyl)tetrazol-5-yl]-[6-methoxy-1-methyl-4-(1-methylpyrazol-4-yl)-3,4-dihydro-1H-isoquinolin-2-yl]methanone as a racemic mixture of syn and anti-isomers as an 82:18 ratio, respectively.

The racemic mixture was further purified by chromatography on a chiral phase, where the major syn-isomers were isolated:

Chiralpak-IG (250×20 mm; 5 μm), Mobile Phase A: TBME (97%), Mobile phase B: EtOH (3%) Isocratic, Flow rate: 17 mL/min, DAD Detection: DAD, Run Time: 40 min, Sample preparation: 350 mg in 6 mL EtOH+4mLTBME, to give syn isomers in rotameric form.

Isomer 1: [2-(2,4-difluorophenyl)tetrazol-5-yl]-[(1S,4S)-6-methoxy-1-methyl-4-(1-methylpyrazol-4-yl)-3,4-dihydro-1H-isoquinolin-2-yl]methanone (Compound P-38, Table P)

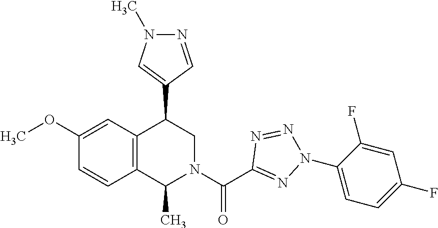 [SMILES: COc1ccc2c(c1)[C@@H](c1cnn(C)c1)CN(C(=O)c1nnn(-c3ccc(F)cc3F)n1)[C@H]2C]

LCMS (Method D): m/z 466 [M+H], retention time 1.50 min.

¹H NMR (400 MHz, CDCl₃) o ppm (Rotamer ratio 65:35) 7.87-7.97 (m, 1H), 7.39-7.43 (m, 1H), 7.31 (s, 1 H), 7.11-7.20 (m, 3H), 6.84 (dd, J=8.57, 2.19 Hz, 1H), 6.60 (d, J=2.50 Hz, 1H), 5.85 (q, J=6.67 Hz, 1H), 4.47 (dd, J=11.63, 5.13 Hz, 1H), 4.25-4.37 (m, 1H), 3.91 (s, 3H), 3.71 (s, 3H), 3.53 (dd, J=13.6, 11.7 Hz, 1 H), 1.68 (d, J=6.75 Hz, 3H)

¹⁹F NMR (376 MHz, CDCl₃) δ-103.71 (s, 1 F), −115.07 (s, 1 F)

### Isomer 2: [2-(2,4-difluorophenyl)tetrazol-5-yl]-[(1R,4R)-6-methoxy-1-methyl-4-(1-methylpyrazol-4-yl)-3,4-dihydro-1H-isoquinolin-2-yl]methanone (Compound P-39, Table P)

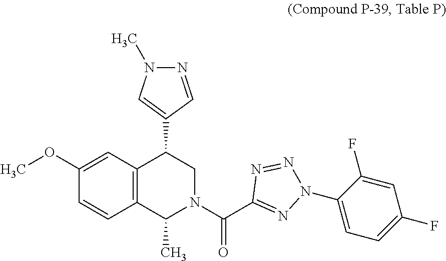 [SMILES: COc1ccc2c(c1)[C@H](c1cnn(C)c1)CN(C(=O)c1nnn(-c3ccc(F)cc3F)n1)[C@@H]2C]

LCMS (Method D): m/z 466 [M+H], retention time 1.50 min.

NMR identical to Isomer 1

### Example P7: Preparation of [6-methoxy-1-methyl-4-(1-methylpyrazol-4-yl)-3,4-dihydro-1H-isoquinolin-2-yl]-(2-phenyltetrazol-5-yl)methanone (Compound P-35, Table P)

Prepared analogously to example 5. Thus, a mixture of 6-methoxy-1-methyl-4-(1-methylpyrazol-4-yl)-1,2,3,4-tetrahydroisoquinoline (0.55 g, 2.14 mmol), 2-phenyltetrazole-5-carboxylic acid (0.45 g, 2.35 mmol), N,N-Diisopropylethylamine (1.17 mL, 6.41 mmol) and T3P (50 mass %) in EtOAc (4.08 g, 3.78 mL, 6.41 mmol) was stirred in Ethyl acetate (11 mL) at rt overnight. LCMS after this time showed the formation of product. The reaction mixture was diluted with water and EtOAc. the EtOAc was decanted and the aqueous backextracted with EtOAc. The combined organic layers were dried over Na₂SO₄, filtered, and concentrated in vacuo. The crude was purified by column to give a disatereomer mixture of the title compound (ratio syn: anti 63:37) as white solid.

Racemic mixture was further purified by Column: Chiral pak-IG (250×20 mm; 5 μm), Mobile Phase A: TBME (80%), Mobile phase B: EtOH (20%) Isocratic, Flow rate: 17 mL/min, DAD Detection: DAD, Run Time: 45 min, Sample preparation: 580 mg in 10 mL EtOH+4 mL THF+1 mL TBME, to give syn isomers in rotameric form.

### Isomer 1: [(1R,4R)-6-methoxy-1-methyl-4-(1-methylpyrazol-4-yl)-3,4-dihydro-1H-isoquinolin-2-yl]-(2-phenyltetrazol-5-yl)methanone (Compound P-36, Table P)

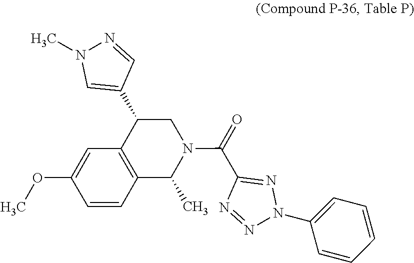 [SMILES: COc1ccc2c(c1)[C@H](c1cnn(C)c1)CN(C(=O)c1nnn(-c3ccccc3)n1)[C@@H]2C]

LCMS (Method D): m/z 430 [M+H], retention time 1.50 min.

¹H NMR (400 MHz, CDCl₃) 0 ppm (Rotamer ratio 64:36) 8.16-8.22 (m, 2H), 7.53-7.62 (m, 3H), 7.40-7.44 (m, 1H), 7.32 (s, 1H), 7.12-7.20 (m, 1H), 6.84 (dd, J=8.57, 2.19 Hz, 1H), 6.58-6.61 (m, 1H), 5.89-5.80 (m, 1H), 4.47 (dd, J=11.57, 5.19 Hz, 1H), 4.33-4.40 (m, 1H), 3.91 (s, 3H), 3.72 (s, 3H), 3.53 (dd, J=13.63, 11.76 Hz, 1H), 1.68 (d, J=6.75 Hz, 3H)

### Isomer 2: [(1S,4S)-6-methoxy-1-methyl-4-(1-methylpyrazol-4-yl)-3,4-dihydro-1H-isoquinolin-2-yl]-(2-phenyltetrazol-5-yl)methanone (Compound P-36, Table P)

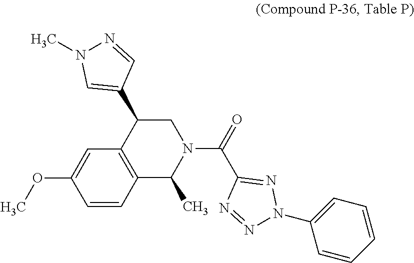 [SMILES: COc1ccc2c(c1)[C@@H](c1cnn(C)c1)CN(C(=O)c1nnn(-c3ccccc3)n1)[C@H]2C]

LCMS (Method D): m/z 430 [M+H], retention time 1.50 min.

¹H NMR (400 MHz, CDCl₃) 0 ppm (Rotamer ratio 61:39) 8.16-8.22 (m, 2H), 7.53-7.62 (m, 3H), 7.40-7.44 (m, 1H), 7.32 (s, 1H), 7.12-7.20 (m, 1H), 6.84 (dd, J=8.57, 2.19 Hz, 1H), 6.58-6.61 (m, 1H), 5.89-5.80 (m, 1H), 4.47 (dd, J=11.57, 5.19 Hz, 1H), 4.33-4.40 (m, 1H), 3.91 (s, 3H), 3.68 (s, 3H), 3.53 (dd, J=13.63, 11.76 Hz, 1H), 1.68 (d, J=6.75 Hz, 3H).

Further examples of synthesized compounds of formula (I) are shown in Table P.

TABLE P
Synthesized compounds and Spectral and Physical Chemical Data.

| | | | RT | [M + H] | |
|---|---|---|---|---|---|
| Entry | IUPAC name | Molecule | (min) | (measured) | Method |
| P-1 | [1-(2-fluorophenyl)pyrazol-3-yl]-[4-(1- methylpyrazol-4-yl)-3,4-dihydro-1H- isoquinolin-2-yl]methanone | 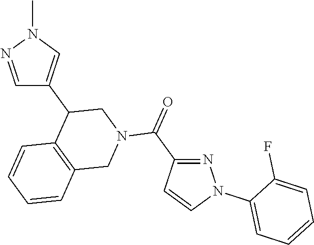 [SMILES: Cn1cc(C2CN(C(=O)c3ccn(-c4ccccc4F)n3)Cc3ccccc32)cn1] | 0.98 | 402 | A |
| P-2 | [1-(2,4-difluorophenyl)pyrazol-3-yl]-[4- (1-methylpyrazol-4-yl)-3,4-dihydro-1H- isoquinolin-2-yl]methanone | 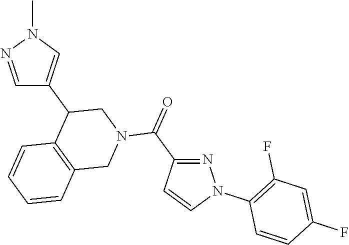 [SMILES: Cn1cc(C2CN(C(=O)c3ccn(-c4ccc(F)cc4F)n3)Cc3ccccc32)cn1] | 0.98 | 420 | A |
| P-3 | [1-(2-fluorophenyl)pyrazol-4-yl]-[4-(1- methylpyrazol-4-yl)-3,4-dihydro-1H- isoquinolin-2-yl]methanone | 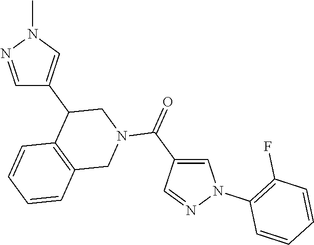 [SMILES: Cn1cc(C2CN(C(=O)c3cnn(-c4ccccc4F)c3)Cc3ccccc32)cn1] | 0.92 | 403 | A |
| P-4 | [2,5-dimethyl-1-(2,2,2- trifluoroethyl)pyrrol-3-yl]-[4-(1- methylpyrazol-4-yl)-3,4-dihydro-1H- isoquinolin-2-yl]methanone | 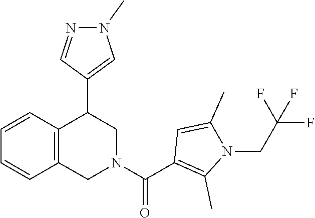 [SMILES: Cc1cc(C(=O)N2Cc3ccccc3C(c3cnn(C)c3)C2)c(C)n1CC(F)(F)F] | 0.98 | 417 | A |
| P-5 | (1,5-dimethylpyrazol-3-yl)-[4-(1- methylpyrazol-4-yl)-3,4-dihydro-1H- isoquinolin-2-yl]methanone | 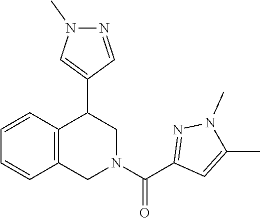 [SMILES: Cc1cc(C(=O)N2Cc3ccccc3C(c3cnn(C)c3)C2)nn1C] | 0.23 | 335 | A |
| P-6 | (2-phenyltriazol-4-yl)-[4-(1,3,5- trimethylpyrazol-4-yl)-3,4-dihydro-1H- isoquinolin-2-yl]methanone | 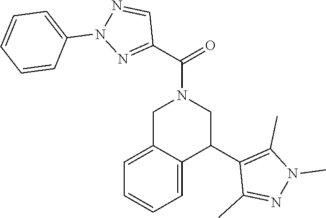 [SMILES: Cc1nn(C)c(C)c1C1CN(C(=O)c2cnn(-c3ccccc3)n2)Cc2ccccc21] | 1.01 | 413 | A |
| P-7 | (1-phenyltriazol-4-yl)-[4-(1,3,5- \|trimethylpyrazol-4-yl)-3,4-dihydro-1H- isoquinolin-2-yl]methanone | 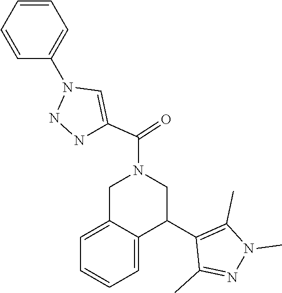 [SMILES: Cc1nn(C)c(C)c1C1CN(C(=O)C2=CN(c3ccccc3)NN2)Cc2ccccc21] | 0.94 | 413 | A |
| P-8 | [1-(2,4-difluorophenyl)triazol-4-yl]-[4-(1- methylpyrazol-4-yl)-3,4-dihydro-1H- isoquinolin-2-yl]methanone | 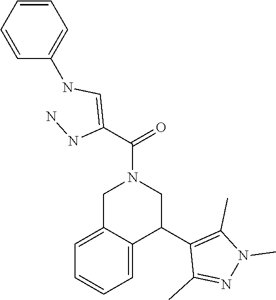 [SMILES: Cc1nn(C)c(C)c1C1CN(C(=O)/C(=C/Nc2ccccc2)NN)Cc2ccccc21] | 0.92 | 421 | A |
| P-9 | [1-(4-fluorophenyl)triazol-4-yl]-[4-(1- methylpyrazol-4-yl)-3,4-dihydro-1H- isoquinolin-2-yl]methanone | 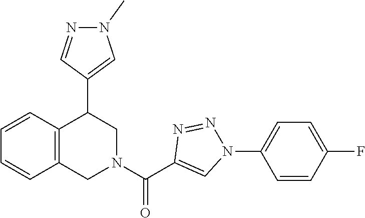 [SMILES: Cn1cc(C2CN(C(=O)c3cn(-c4ccc(F)cc4)nn3)Cc3ccccc32)cn1] | 1.21 | 403 | E |
| P-10 | 1H-isoquinolin-2-yl]-(1-phenylimidazol- [4-(1-methylpyrazol-4-yl)-3,4-dihydro- 4-yl)methanone | 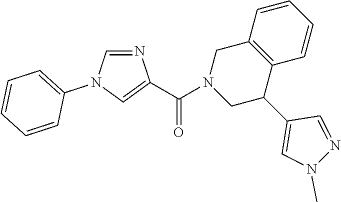 [SMILES: Cn1cc(C2CN(C(=O)c3cn(-c4ccccc4)cn3)Cc3ccccc32)cn1] | 1.27 | 384 | E |
| P-11 | [4-(1-methylpyrazol-4-yl)-3,4-dihydro- 1H-isoquinolin-2-yl]-(1-phenyltriazol-4- yl)methanone | 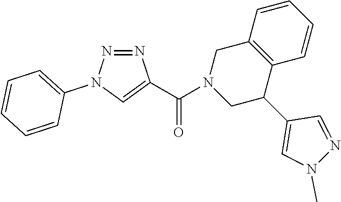 [SMILES: Cn1cc(C2CN(C(=O)c3cn(-c4ccccc4)nn3)Cc3ccccc32)cn1] | 1.19 | 385 | E |
| P-12 | [2,5-dimethyl-1-(5-methylisoxazol-3- yl)pyrrol-3-yl]-[4-(1-methylpyrazol-4-yl)- 3,4-dihydro-1H-isoquinolin-2- yl]methanone | 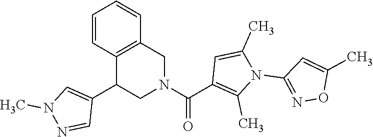 | 1.39 | 416 | E |
| P-13 | (1-benzyltriazol-4-yl)-[4-(1- methylpyrazol-4-yl)-3,4-dihydro-1H- isoquinolin-2-yl]methanone | 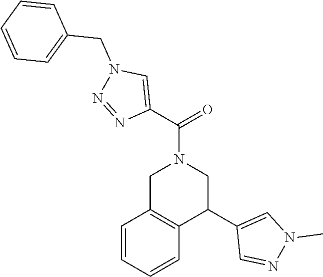 [SMILES: Cn1cc(C2CN(C(=O)c3cn(Cc4ccccc4)nn3)Cc3ccccc32)cn1] | 1.29 | 399 | E |
| P-14 | [4-(1-methylpyrazol-4-yl)-3,4-dihydro- 1H-isoquinolin-2-yl]-(2-phenyltetrazol- 5-yl)methanone | 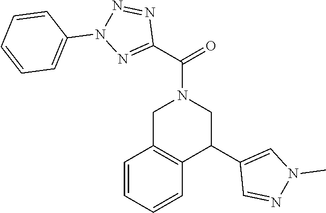 [SMILES: Cn1cc(C2CN(C(=O)c3nnn(-c4ccccc4)n3)Cc3ccccc32)cn1] | 1.35 | 386 | E |
| P-15 | (1-isopropyltriazol-4-yl)-[4-(1- methylpyrazol-4-yl)-3,4-dihydro-1H- isoquinolin-2-yl]methanone | 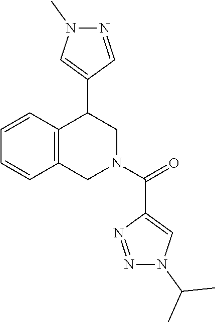 [SMILES: CC(C)n1cc(C(=O)N2Cc3ccccc3C(c3cnn(C)c3)C2)nn1] | 1.18 | 351 | E |
| P-16 | 1H-isoquinolin-2-yl]-(1-phenylpyrazol- [4-(1-methylpyrazol-4-yl)-3,4-dihydro- 3-yl)methanone | 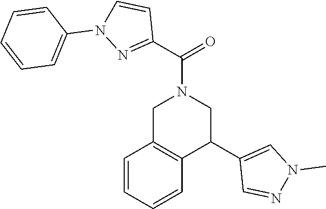 [SMILES: Cn1cc(C2CN(C(=O)c3ccn(-c4ccccc4)n3)Cc3ccccc32)cn1] | 1.33 | 384 | E |
| P-17 | [1-(4-fluorophenyl)-5-methyl-triazol-4- yl]-[4-(1-methylpyrazol-4-yl)-3,4- dihydro-1H-isoquinolin-2-yl]methanone | 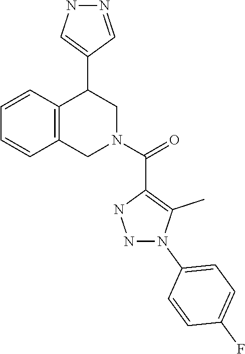 [SMILES: CC1=C(C(=O)N2Cc3ccccc3C(c3cn[nH]c3)C2)NNN1c1ccc(F)cc1] | 1.34 | 417 | E |
| P-18 | [2-(4-fluorophenyl)triazol-4-yl]-[4-(1- methylpyrazol-4-yl)-3,4-dihydro-1H- isoquinolin-2-yl]methanone | 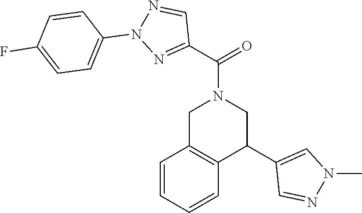 [SMILES: Cn1cc(C2CN(C(=O)c3cnn(-c4ccc(F)cc4)n3)Cc3ccccc32)cn1] | 1.41 | 403 | E |
| P-19 | [4-(1-methylpyrazol-4-yl)-3,4-dihydro- 1H-isoquinolin-2-yl]-(1-phenyl-1,2,4- triazol-3-yl)methanone | 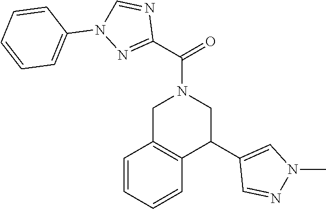 [SMILES: Cn1cc(C2CN(C(=O)c3ncn(-c4ccccc4)n3)Cc3ccccc32)cn1] | 1.24 | 385 | E |
| P-20 | (1-tert-butylimidazol-4-yl)-[4-(1- methylpyrazol-4-yl)-3,4-dihydro-1H- isoquinolin-2-yl]methanone | 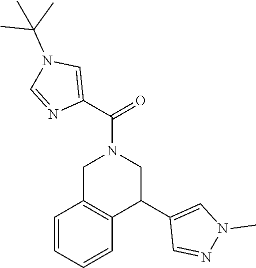 [SMILES: Cn1cc(C2CN(C(=O)c3cn(C(C)(C)C)cn3)Cc3ccccc32)cn1] | 1.06 | 364 | E |
| P-21 | [1-(4-fluorophenyl)pyrazol-3-yl]-[4-(1- methylpyrazol-4-yl)-3,4-dihydro-1H- isoquinolin-2-yl]methanone | 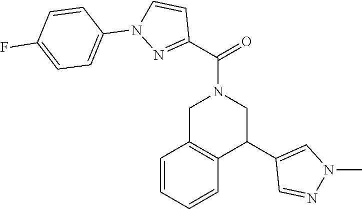 [SMILES: Cn1cc(C2CN(C(=O)c3ccn(-c4ccc(F)cc4)n3)Cc3ccccc32)cn1] | 1.40 | 402 | E |
| P-22 | (5-cyclopropyl-1-methyl-pyrazol-3-yl)- [4-(1-methylpyrazol-4-yl)-3,4-dihydro- 1H-isoquinolin-2-yl]methanone | 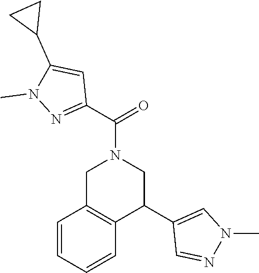 | 1.28 | 362 | E |
| P-23 | [1-(3-fluoro-2-pyridyl)-1,2,4-triazol-3- yl]-[4-(1-methylpyrazol-4-yl)-3,4- dihydro-1H-isoquinolin-2-yl]methanone | 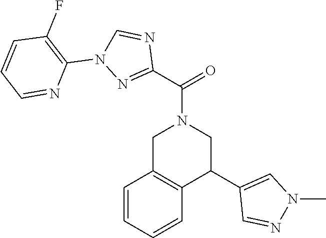 [SMILES: Cn1cc(C2CN(C(=O)c3ncn(-c4ncccc4F)n3)Cc3ccccc32)cn1] | 1.70 | 404 | E |
| P-24 | [1-(3-fluorophenyl)-3-methyl-pyrazol-4- yl]-[4-(1-methylpyrazol-4-yl)-3,4- dihydro-1H-isoquinolin-2-yl]methanone | 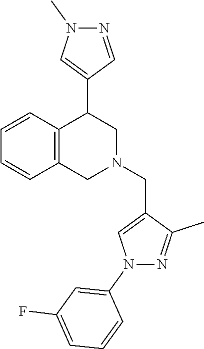 [SMILES: Cc1nn(-c2cccc(F)c2)cc1CN1Cc2ccccc2C(c2cnn(C)c2)C1] | 1.39 | 416 | E |
| P-25 | (5-benzyl-1-methyl-pyrazol-3-yl)-[4-(1- methylpyrazol-4-yl)-3,4-dihydro-1H- isoquinolin-2-yl]methanone | 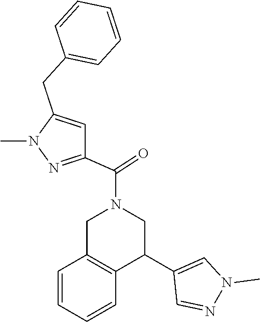 | 1.38 | 412 | E |
| P-26 | [4-(1-methylpyrazol-4-yl)-3,4-dihydro- 1H-isoquinolin-2-yl]-[1-[5- (trifluoromethyl)-2-pyridyl]triazol-4- yl]methanone | 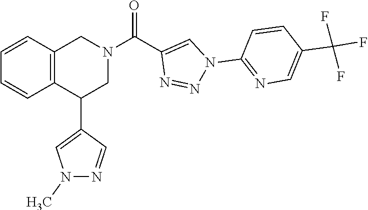 [SMILES: Cn1cc(C2CN(C(=O)c3cn(-c4ccc(C(F)(F)F)cn4)nn3)Cc3ccccc32)cn1] | 1.27 | 454 | E |
| P-27 | [1-(2-methoxyphenyl)imidazol-4-yl]-[4- (1-methylpyrazol-4-yl)-3,4-dihydro-1H- isoquinolin-2-yl]methanone | 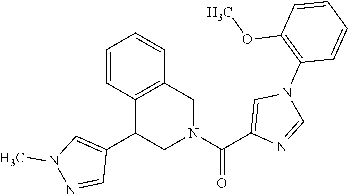 [SMILES: COc1ccccc1-n1cnc(C(=O)N2Cc3ccccc3C(c3cnn(C)c3)C2)c1] | 1.20 | 414 | E |
| P-28 | (5-cyclopropyl-1-ethyl-pyrazol-3-yl)-[4- (1-methylpyrazol-4-yl)-3,4-dihydro-1H- isoquinolin-2-yl]methanone | 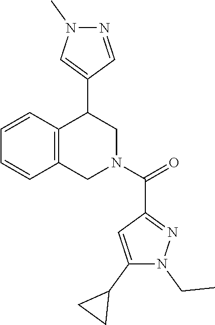 [SMILES: CCn1nc(C(=O)N2Cc3ccccc3C(c3cnn(C)c3)C2)cc1C1CC1] | 1.32 | 376 | E |
| P-29 | (1-methylpyrazol-4-yl)-3,4-dihydro-1H- [1-(2,6-difluorophenyl)pyrazol-3-yl]-[4- isoquinolin-2-yl]methanone | 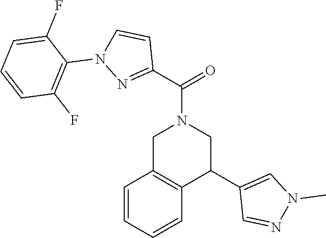 [SMILES: Cn1cc(C2CN(C(=O)c3ccn(-c4c(F)cccc4F)n3)Cc3ccccc32)cn1] | 1.36 | 420 | E |
| P-30 | (1-methylpyrazol-4-yl)-3,4-dihydro-1H- [1-(3,4-difluorophenyl)pyrazol-3-yl]-[4- isoquinolin-2-yl]methanone | 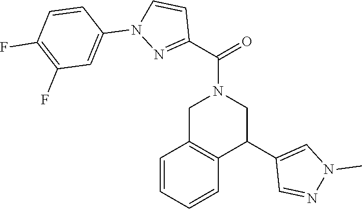 [SMILES: Cn1cc(C2CN(C(=O)c3ccn(-c4ccc(F)c(F)c4)n3)Cc3ccccc32)cn1] | 1.42 | 420 | E |
| P-31 | [2-(2,4-difluorophenyl)tetrazol-5-yl]-[4- (1,5-dimethylpyrazol-4-yl)-3,4-dihydro- 1H-isoquinolin-2-yl]methanone | 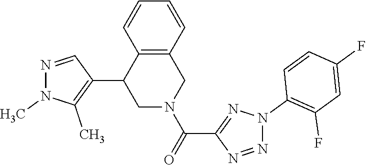 [SMILES: Cc1c(C2CN(C(=O)c3nnn(-c4ccc(F)cc4F)n3)Cc3ccccc32)cnn1C] | 1.07 | 436 | G |
| P-32 | [2-(2,4-difluorophenyl)tetrazol-5-yl]-[4- methyl-4-(1-methylpyrazol-4-yl)-1,3- dihydroisoquinolin-2-yl]methanone | 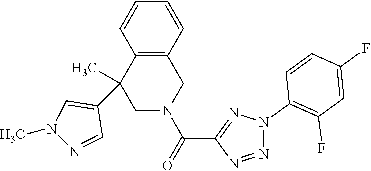 [SMILES: Cn1cc(C2(C)CN(C(=O)c3nnn(-c4ccc(F)cc4F)n3)Cc3ccccc32)cn1] | 1.06 | 436 | G |
| P-33 | [1-(2,4-difluorophenyl)-1,2,4-triazol-3- yl]-[4-(1,5-dimethylpyrazol-4-yl)-3,4- dihydro-1H-isoquinolin-2-yl]methanone | 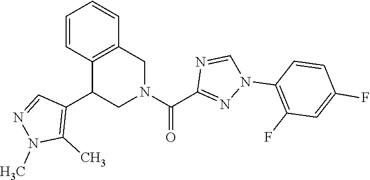 [SMILES: Cc1c(C2CN(C(=O)c3ncn(-c4ccc(F)cc4F)n3)Cc3ccccc32)cnn1C] | 1.38 | 435 | F |
| P-34 | [2-(2,4-difluorophenyl)tetrazol-5-yl]-[6- methoxy-1-methyl-4-(1-methylpyrazol- 4-yl)-3,4-dihydro-1H-isoquinolin-2- yl]methanone | 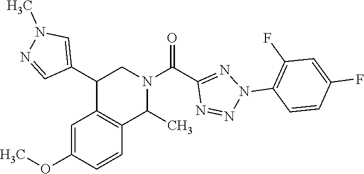 [SMILES: COc1ccc2c(c1)C(c1cnn(C)c1)CN(C(=O)c1nnn(-c3ccc(F)cc3F)n1)C2C] | 1.50 | 466 | F |
| P-35 | [6-methoxy-1-methyl-4-(1- methylpyrazol-4-yl)-3,4-dihydro-1H- isoquinolin-2-yl]-(2-phenyltetrazol-5- yl)methanone | 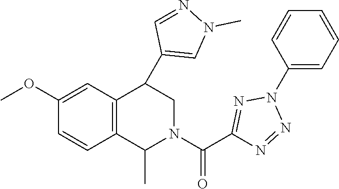 [SMILES: COc1ccc2c(c1)C(c1cnn(C)c1)CN(C(=O)c1nnn(-c3ccccc3)n1)C2C] | 1.52 | 430 | F |
| P-36 | [(1R,4R)-6-methoxy-1-methyl-4-(1- methylpyrazol-4-yl)-3,4-dihydro-1H- isoquinolin-2-yl]-(2-phenyltetrazol-5- yl)methanone | 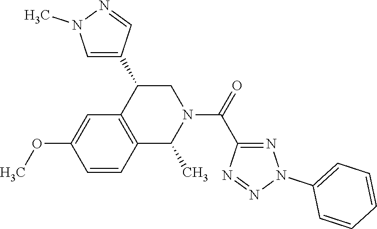 [SMILES: COc1ccc2c(c1)[C@H](c1cnn(C)c1)CN(C(=O)c1nnn(-c3ccccc3)n1)[C@@H]2C] | 1.50 | 430 | F |
| P-37 | [(1S,4S)-6-methoxy-1-methyl-4-(1- methylpyrazol-4-yl)-3,4-dihydro-1H- isoquinolin-2-yl]-(2-phenyltetrazol-5- yl)methanone | 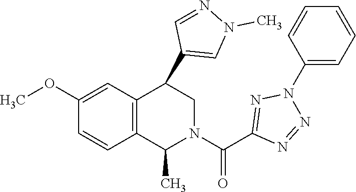 [SMILES: COc1ccc2c(c1)[C@@H](c1cnn(C)c1)CN(C(=O)c1nnn(-c3ccccc3)n1)[C@H]2C] | 1.50 | 430 | F |
| P-38 | [2-(2,4-difluorophenyl)tetrazol-5-yl]- [(1S,4S)-6-methoxy-1-methyl-4-(1- methylpyrazol-4-yl)-3,4-dihydro-1H- isoquinolin-2-yl]methanone | 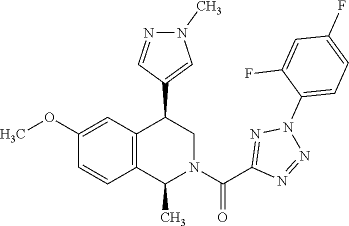 [SMILES: COc1ccc2c(c1)[C@@H](c1cnn(C)c1)CN(C(=O)c1nnn(-c3ccc(F)cc3F)n1)[C@H]2C] | 1.50 | 466 | F |
| P-39 | [2-(2,4-difluorophenyl)tetrazol-5-yl]- [(1R,4R)-6-methoxy-1-methyl-4-(1- methylpyrazol-4-yl)-3,4-dihydro-1H- isoquinolin-2-yl]methanone | 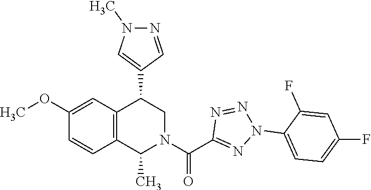 [SMILES: COc1ccc2c(c1)[C@H](c1cnn(C)c1)CN(C(=O)c1nnn(-c3ccc(F)cc3F)n1)[C@@H]2C] | 1.50 | 466 | F |
| P-40 | [2-(2,4-difluorophenyl)triazol-4-yl]-[rac- (1S,4S)-4-(1,5-dimethylpyrazol-4-yl)-1- methyl-3,4-dihydro-1H-isoquinolin-2- yl]methanone | 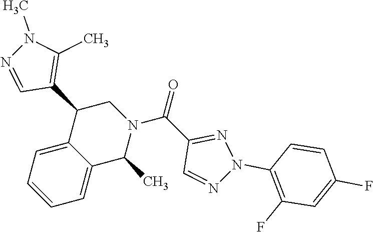 [SMILES: Cc1c([C@H]2CN(C(=O)c3cnn(-c4ccc(F)cc4F)n3)[C@@H](C)c3ccccc32)cnn1C] | 1.14 | 449 | G |
| P-41 | [1-(2,4-difluorophenyl)triazol-4-yl]-[rac- (1S,4S)-4-(1,5-dimethylpyrazol-4-yl)-1- methyl-3,4-dihydro-1H-isoquinolin-2- yl]methanone | 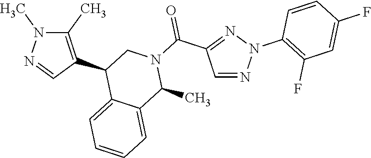 [SMILES: Cc1c([C@H]2CN(C(=O)c3cnn(-c4ccc(F)cc4F)n3)[C@@H](C)c3ccccc32)cnn1C] | 1.13 | 449 | G |
| P-42 | [2-(2,4-difluorophenyl)tetrazol-5-yl]- [rac-(1S,4S)-4-(1,5-dimethylpyrazol-4- yl)-1-methyl-3,4-dihydro-1H- isoquinolin-2-yl]methanone | 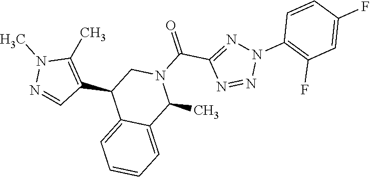 [SMILES: Cc1c([C@H]2CN(C(=O)c3nnn(-c4ccc(F)cc4F)n3)[C@@H](C)c3ccccc32)cnn1C] | 1.12 | 450 | G |
| P-43 | [1-(3,5-difluoro-2-pyridyl)triazol-4-yl]- [(1S,4S)-4-(1,5-dimethylpyrazol-4-yl)-1- methyl-3,4-dihydro-1H-isoquinolin-2- yl]methanone | 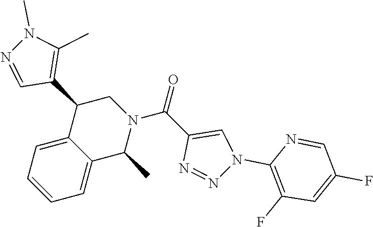 [SMILES: Cc1c([C@H]2CN(C(=O)c3cn(-c4ncc(F)cc4F)nn3)[C@@H](C)c3ccccc32)cnn1C] | 1.06 | 450 | G |
| P-44 | [1-(3,5-difluoro-2-pyridyl)triazol-4-yl]- [(1R,4R)-4-(1,5-dimethylpyrazol-4-yl)- 1-methyl-3,4-dihydro-1H-isoquinolin-2- yl]methanone | 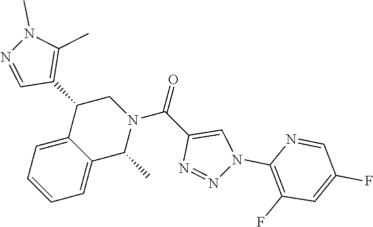 [SMILES: Cc1c([C@@H]2CN(C(=O)c3cn(-c4ncc(F)cc4F)nn3)[C@H](C)c3ccccc32)cnn1C] | 1.05 | 450 | G |
| P-45 | [(1S,4S)-4-(5-chloro-1-methyl-pyrazol- 4-yl)-1-methyl-3,4-dihydro-1H- isoquinolin-2-yl]-[2-(2,4- difluorophenyl)triazol-4-yl]methanone | 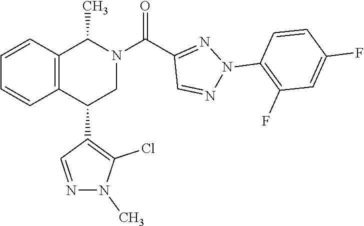 [SMILES: C[C@H]1c2ccccc2[C@@H](c2cnn(C)c2Cl)CN1C(=O)c1cnn(-c2ccc(F)cc2F)n1] | 1.23 | 469 | G |
| P-46 | [(1R,4R)-4-(5-chloro-1-methyl-pyrazol- 4-yl)-1-methyl-3,4-dihydro-1H- isoquinolin-2-yl]-[2-(2,4- difluorophenyl)triazol-4-yl]methanone | 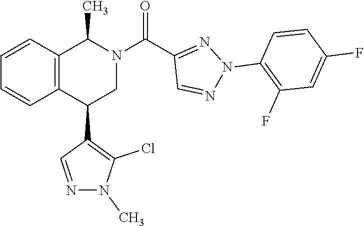 [SMILES: C[C@@H]1c2ccccc2[C@H](c2cnn(C)c2Cl)CN1C(=O)c1cnn(-c2ccc(F)cc2F)n1] | 1.23 | 469 | G |
| P-47 | [(1R,4R)-4-(5-chloro-1-methyl-pyrazol- 4-yl)-1-methyl-3,4-dihydro-1H- isoquinolin-2-yl]-[1-(2,4- difluorophenyl)triazol-4-yl]methanone | 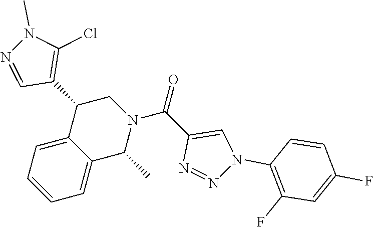 [SMILES: C[C@@H]1c2ccccc2[C@H](c2cnn(C)c2Cl)CN1C(=O)c1cn(-c2ccc(F)cc2F)nn1] | 1.18 | 469 | G |
| P-48 | [(1S,4S)-4-(5-chloro-1-methyl-pyrazol- 4-yl)-1-methyl-3,4-dihydro-1H- isoquinolin-2-yl]-[1-(2,4- difluorophenyl)triazol-4-yl]methanone | 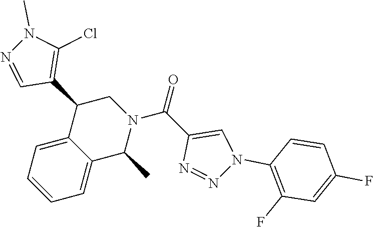 [SMILES: C[C@H]1c2ccccc2[C@@H](c2cnn(C)c2Cl)CN1C(=O)c1cn(-c2ccc(F)cc2F)nn1] | 1.18 | 469 | G |
| P-49 | [(1S,4S)-4-(5-chloro-1-methyl-pyrazol- 4-yl)-1-methyl-3,4-dihydro-1H- isoquinolin-2-yl]-[2-(2,4- difluorophenyl)tetrazol-5-yl]methanone | 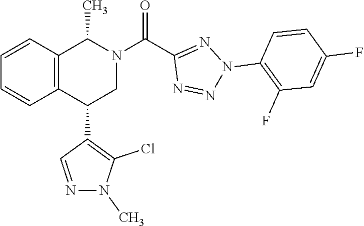 [SMILES: C[C@H]1c2ccccc2[C@@H](c2cnn(C)c2Cl)CN1C(=O)c1nnn(-c2ccc(F)cc2F)n1] | 1.05 | 470 | G |
| P-50 | [(1R,4R)-4-(5-chloro-1-methyl-pyrazol- 4-yl)-1-methyl-3,4-dihydro-1H- isoquinolin-2-yl]-[2-(2,4- difluorophenyl)tetrazol-5-yl]methanone | 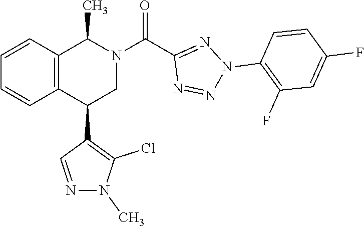 [SMILES: C[C@@H]1c2ccccc2[C@H](c2cnn(C)c2Cl)CN1C(=O)c1nnn(-c2ccc(F)cc2F)n1] | 1.05 | 470 | G |
| P-51 | [1-(3,5-difluoro-2-pyridyl)triazol-4-yl]- [(1S,4S)-1-methyl-4-(1-methylpyrazol- 4-yl)-3,4-dihydro-1H-isoquinolin-2- yl]methanone | 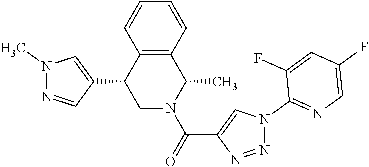 [SMILES: C[C@H]1c2ccccc2[C@@H](c2cnn(C)c2)CN1C(=O)c1cn(-c2ncc(F)cc2F)nn1] | 1.07 | 436 | G |
| P-52 | [1-(3,5-difluoro-2-pyridyl)triazol-4-yl]- [(1R,4R)-1-methyl-4-(1-methylpyrazol- 4-yl)-3,4-dihydro-1H-isoquinolin-2- yl]methanone | 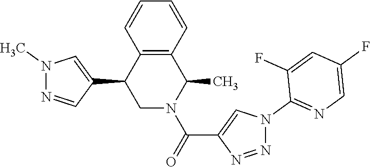 [SMILES: C[C@@H]1c2ccccc2[C@H](c2cnn(C)c2)CN1C(=O)c1cn(-c2ncc(F)cc2F)nn1] | 1.07 | 436 | G |
| P-53 | [1-(3,5-difluoro-2-pyridyl)triazol-4-yl]- [rac-(1S,4S)-4-(5-chloro-1-methyl- pyrazol-4-yl)-1-methyl-3,4-dihydro-1H- isoquinolin-2-yl]methanone | 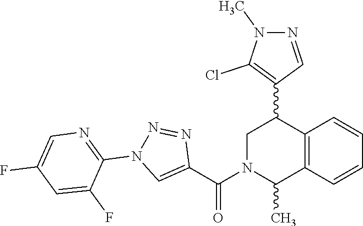 [SMILES: CC1c2ccccc2C(c2cnn(C)c2Cl)CN1C(=O)c1cn(-c2ncc(F)cc2F)nn1] | 1.17 | 470 | G |
| P-54 | [(1S,4S)-4-(5-chloro-1-methyl-pyrazol- 4-yl)-1-methyl-3,4-dihydro-1H- isoquinolin-2-yl]-[1-(3,5-difluoro-2- pyridyl)triazol-4-yl]methanone | 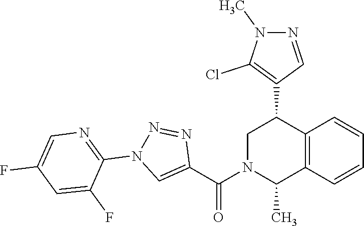 [SMILES: C[C@H]1c2ccccc2[C@@H](c2cnn(C)c2Cl)CN1C(=O)c1cn(-c2ncc(F)cc2F)nn1] | 1.15 | 470 | G |
| P-55 | [(1R,4R)-4-(5-chloro-1-methyl-pyrazol- 4-yl)-1-methyl-3,4-dihydro-1H- isoquinolin-2-yl]-[1-(3,5-difluoro-2- pyridyl)triazol-4-yl]methanone | 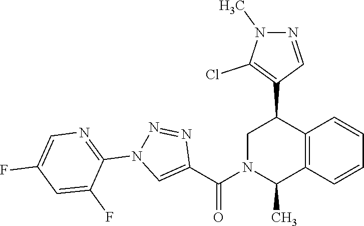 [SMILES: C[C@@H]1c2ccccc2[C@H](c2cnn(C)c2Cl)CN1C(=O)c1cn(-c2ncc(F)cc2F)nn1] | 1.15 | 470 | G |
| P-56 | [1-(2,4-difluorophenyl)triazol-4-yl]-[4- (1,5-dimethylpyrazol-4-yl)-3,4-dihydro- 1H-isoquinolin-2-yl]methanone | 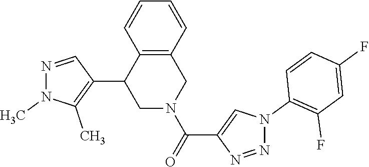 [SMILES: Cc1c(C2CN(C(=O)c3cn(-c4ccc(F)cc4F)nn3)Cc3ccccc32)cnn1C] | 1.77 | 435 | H |
| P-57 | (1,5-dimethylpyrazol-4-yl)-3,4-dihydro- [1-(3,5-difluoro-2-pyridyl)triazol-4-yl]-[4- 1H-isoquinolin-2-yl]methanone | 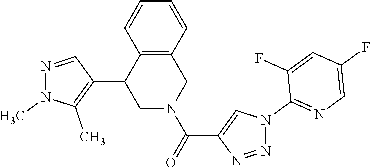 [SMILES: Cc1c(C2CN(C(=O)c3cn(-c4ncc(F)cc4F)nn3)Cc3ccccc32)cnn1C] | 1.67 | 436 | H |

### Example B1: Alternaria solani/Tomato/Leaf Disc (Early Blight)

Tomato leaf disks cv. Baby are placed on agar in multiwell plates (24-well format) and sprayed with the formulated test compound diluted in water. The leaf disks are inoculated with a spore suspension of the fungus 2 days after application. The inoculated leaf disks are incubated at 23° C./21° C. (day/night) and 80% rh under a light regime of 12/12 h (light/dark) in a climate cabinet and the activity of a compound is assessed as percent disease control compared to untreated when an appropriate level of disease damage appears on untreated check disk leaf disks (5-7 days after application).

The following compounds gave at least 80% control of Alternaria solani at 200 ppm when compared to untreated control under the same conditions, which showed extensive disease development: P-14, P-31, P-41, P-42, P-43, P-48, P-49, P-51, P-53, P-54, P-56, P-57

### Example B2: Botryotinia fuckeliana (Botrytis cinerea)/Liquid Culture (Gray Mould)

Conidia of the fungus from cryogenic storage are directly mixed into nutrient broth (Vogels broth). After placing a (DMSO) solution of test compound into a microtiter plate (96-well format), the nutrient broth containing the fungal spores is added. The test plates are incubated at 24° C. and the inhibition of growth is determined photometrically 3-4 days after application.

The following compounds gave at least 80% control of Botryotinia fuckeliana at 20 ppm when compared to untreated control under the same conditions, which showed extensive disease development: P-14, P-31, P-35, P-37, P-41, P-42, P-43, P-48, P-49, P-51, P-53, P-54, P-56

### Example B3: Glomerella lagenarium (Colletotrichum lagenarium)/liquid culture (Anthracnose)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is measured photometrically 3-4 days after application.

The following compounds gave at least 80% control of Glomerella lagenarium at 20 ppm when compared to untreated control under the same conditions, which showed extensive disease development: P-11, P-14, P-31, P-35, P-41, P-42, P-43, P-44, P-48, P-49, P-51, P-53, P-54, P-56

### Example B4: Blumeria graminis f. sp. tritici (Erysiphe graminis f. sp. tritici)/Wheat/Leaf Disc Preventative

### (Powdery Mildew on Wheat)

Wheat leaf segments cv. Kanzler are placed on agar in a multiwell plate (24-well format) and sprayed with the formulated test compound diluted in water. The leaf disks are inoculated by shaking powdery mildew infected plants above the test plates 1 day after application. The inoculated leaf disks are incubated at 20° C. and 60% rh under a light regime of 24 h darkness followed by 12 h light/12 h darkness in a climate chamber and the activity of a compound is assessed as percent disease control compared to untreated when an appropriate level of disease damage appears on untreated check leaf segments (6-8 days after application). The following compounds gave at least 80% control of Blumeria graminis f. sp. tritici at 200 ppm when compared to untreated control under the same conditions, which showed extensive disease development: P-37, P-42, P-43, P-48, P-49, P-51, P-53, P-54, P-56

### Example B5: Fusarium culmorum/Liquid Culture (Head Blight)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 3-4 days after application.

The following compounds gave at least 80% control of Fusarium culmorum at 20 ppm when compared to untreated control under the same conditions, which showed extensive disease development: P-42, P-43, P-48, P-53, P-54

### Example B6: Gibberella zeae (Fusarium graminearum)/Wheat/Spikelet Preventative (Head Blight)

Wheat spikelets cv. Monsun are placed on agar in multiwell plates (24-well format) and sprayed with the formulated test compound diluted in water. One day after application, the spikelets are inoculated with a spore suspension of the fungus. The inoculated test leaf disks are incubated at 20° C. and 60% rh under a light regime of 72 h semi darkness followed by 12 h light/12 h darkness in a climate chamber, the activity of a compound is assessed as percent disease control compared to untreated when an appropriate level of disease damage appears on untreated check spikelets (6-8 days after application).

The following compounds gave at least 80% control of Gibberella zeae at 200 ppm when compared to untreated control under the same conditions, which showed extensive disease development: P-54

### Example B7: Phaeosphaeria Nodorum (Septoria nodorum)/Wheat/Leaf Disc Preventative (Glume Blotch)

Wheat leaf segments cv. Kanzler are placed on agar in a multiwell plate (24-well format) and sprayed with the formulated test compound diluted in water. The leaf disks are inoculated with a spore suspension of the fungus 2 days after application. The inoculated test leaf disks are incubated at 20° C. and 75% rh under a light regime of 12 h light/12 h darkness in a climate cabinet and the activity of a compound is assessed as percent disease control compared to untreated when an appropriate level of disease damage appears in untreated check leaf disks (5-7 days after application).

The following compounds gave at least 80% control of Phaeosphaeria nodorum at 200 ppm when compared to untreated control under the same conditions, which showed extensive disease development: P-14, P-31, P-37, P-41, P-42, P-43, P-48, P-49, P-51, P-53, P-54, P-56, P-57

### Example B8: Monographella nivalis (Microdochium nivale)/Liquid Culture (Foot Rot Cereals)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Monographella nivalis at 20 ppm when compared to untreated control under the same conditions, which showed extensive disease development: P-14, P-18, P-31, P-34, P-35, P-36, P-37, P-38, P-41, P-42, P-43, P-48, P-49, P-51, P-53, P-54, P-56

### Example B9: Mycosphaerella arachidis (Cercospora arachidicola)/Liquid Culture (Early Leaf Spot)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Mycosphaerella arachidis at 20 ppm when compared to untreated control under the same conditions, which showed extensive disease development: P-7, P-14, P-31, P-34, P-35, P-36, P-37, P-38, P-41, P-42, P-43, P-44, P-47, P-48, P-49, P-51, P-53, P-54, P-56

### Example B10: Puccinia recondita f. Sp. Tritici/Wheat/Leaf Disc Curative (Brown Rust)

Wheat leaf segments cv. Kanzler are placed on agar in multiwell plates (24-well format). The leaf segments are inoculated with a spore suspension of the fungus. Plates are stored in darkness at 19° C. and 75% rh. The formulated test compound diluted in water is applied 1 day after inoculation. The leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (6-8 days after application).

The following compounds gave at least 80% control of Puccinia recondita f. sp. tritici at 200 ppm when compared to untreated control under the same conditions, which showed extensive disease development: P-42

### Example B11: Puccinia recondita f. Sp. tritici/Wheat/Leaf Disc Preventative (Brown Rust)

Wheat leaf segments cv. Kanzler are placed on agar in multiwell plates (24-well format) and sprayed with the formulated test compound diluted in water. The leaf disks are inoculated with a spore suspension of the fungus 1 day after application. The inoculated leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (7-9 days after application).

The following compounds gave at least 80% control of Puccinia recondita f. sp. tritici at 200 ppm when compared to untreated control under the same conditions, which showed extensive disease development: P-37, P-41, P-42, P-49

### Example B12: Pyrenophora teres/Barley/Leaf Disc Preventative (Net Blotch)

Barley leaf segments cv. Hasso are placed on agar in a multiwell plate (24-well format) and sprayed with the formulated test compound diluted in water. The leaf segments are inoculated with a spore suspension of the fungus 2 days after application. The inoculated leaf segments are incubated at 20° C. and 65% rh under a light regime of 12 h light/12 h darkness in a climate cabinet and the activity of a compound is assessed as disease control compared to untreated when an appropriate level of disease damage appears in untreated check leaf segments (5-7 days after application).

The following compounds gave at least 80% control of Pyrenophora teres at 200 ppm when compared to untreated control under the same conditions, which showed extensive disease development: P-8, P-14, P-31, P-35, P-37, P-40, P-41, P-42, P-43, P-48, P-49, P-51, P-53, P-54, P-56, P-57

### Example B13: Sclerotinia sclerotiorum/Liquid Culture (Cottony Rot)

Mycelia fragments of a newly grown liquid culture of the fungus are directly mixed into nutrient broth (PDB potato dextrose broth). After placing a (DMSO) solution of test compound into a microtiter plate (96-well format) the nutrient broth containing the fungal material is added. The test plates are incubated at 24° C. and the inhibition of growth is determined photometrically 3-4 days after application.

The following compounds gave at least 80% control of Sclerotinia sclerotiorum at 20 ppm when compared to untreated control under the same conditions, which showed extensive disease development: P-42, P-48

### Example B14: Mycosphaerella graminicola (Septoria tritici)/Liquid Culture (Septoria Blotch)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Mycosphaerella graminicola at 20 ppm when compared to untreated control under the same conditions, which showed extensive disease development: P-7, P-11, P-14, P-18, P-31, P-32, P-34, P-35, P-36, P-37, P-38, P-40, P-41, P-42, P-43, P-44, P-45, P-47, P-48, P-49, P-50, P-51, P-53, P-54, P-56, P-57

1. A compound of formula (I): 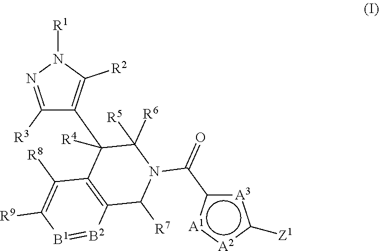
wherein
R¹ is selected from hydrogen, C₁-C₄-alkyl, C₂-C₄-alkenyl, C₂-C₄-alkynyl, or C₃-C₆cycloalkyl;
R² is selected from hydrogen, halogen, C₁-C₄-alkyl, C₂-C₄-alkenyl, C₂-C₄-alkynyl, C₁-C₄-haloalkyl, C₃-C₆cycloalkyl, C₁-C₄-alkylcarbonyl, N—C₁-C₄alkoxy-C-C₁-C₄alkyl-carbonimidoyl, N-hydroxy-C-C₁-C₄alkyl-carbonimidoyl, or C₁-C₄ alkoxycarbonyl;
R³ and R⁴ are independently selected from hydrogen, halogen, or C₁-C₄-alkyl;
R⁵ and R⁶ are independently selected from hydrogen, or C₁-C₄alkyl;
R⁷ is selected from hydrogen, C₁-C₄alkyl, C₁-C₄ alkylcarbonyl, N—C₁-C₄ alkoxy-C-C₁-C₄ alkyl-carbonimidoyl, N-hydroxy-C-C₁-C₄ alkyl-carbonimidoyl, C₁-C₄alkoxycarbonyl, N-methoxy-N-methyl-carbonyl, C₁-C₄ alkylaminocarbonyl, di(C₁-C₆ alkylamino) carbonyl, phenyl, a 5- to 6-membered heteroaryl, or C₃-C₆-cycloalkyl; wherein said 5- or 6-membered heteroaryl comprises 1, 2, 3, or 4 heteroatoms individually selected from N, O, or S and wherein said phenyl and 5- to 6-membered heteroaryl are unsubstituted or substituted with 1, 2, or 3 substituents independently selected from halogen, C₁-C₄ haloalkyl, cyano, carboxy, C₁-C₄ alkyl, or C₁-C₄ alkoxy; and wherein said C₃-C₆-cycloalkyl is unsubstituted or substituted with 1, 2, or 3 substituents independently selected from halogen, C₁-C₄ haloalkyl, cyano, C₁-C₄ alkyl, or C₁-C₄ alkoxy;
B¹ is selected from CR¹⁰, or N;
B² is selected from CR¹¹, or N;
R⁸, R⁹, R¹⁰ and R¹¹ are independently selected from hydrogen, halogen, C₁-C₄alkyl, C₁-C₄haloalkyl, C₁-C₄alkoxy, C₁-C₄ haloalkoxy, C₂-C₄alkenyloxy, C₂-C₄alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-4alkylamino, N,N-diC₁₋₄alkylamino, C₁-C₆ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl, wherein said 5- or 6-membered heteroaryl comprises 1, 2, 3, or 4 heteroatoms individually selected from N, O, or S, and wherein any of said phenyl, 5- to 6-membered heteroaryl and C₃-C₆-cycloalkyl are unsubstituted or substituted by 1, 2, or 3 substituents independently selected from halogen, cyano, C₁-C₄alkyl, C₁-C₄haloalkyl, or C₁-C₄alkoxy;
A¹ is selected from CR^{12a}, or N,
A² is selected from CR^{13a}, or N;
A³ is selected from CR^{14a}, or N;
R^{12a}, R^{13a} and R^{14a} are independently selected from hydrogen, halogen, C₁-C₄alkyl, C₁-C₄haloalkyl, C₂-C₄alkenyl, or C₂-C₄alkynyl; and
Z¹ is selected from C₁-C₄ alkyl, phenyl, 5- or 6-membered heteroaryl, or C₃-C₆ cycloalkyl, wherein said 5- or 6-membered heteroaryl comprises 1, 2, 3, or 4 heteroatoms individually selected from N, O, or S, and wherein any of said phenyl, 5- or 6-membered heteroaryl are unsubstituted or substituted by 1, 2, or 3 substituents independently selected from halogen, cyano, C₁-C₄ alkyl, C₂-C₄ alkenyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl, or C₂-C₄ alkynyl and wherein said C₃-C₆ cycloalkyl is unsubstituted or substituted by 1, 2, or 3 substituents independently selected from halogen, cyano, C₁-C₄ alkyl, C₁-C₄ haloalkyl, or C₁-C₄alkoxy;
or an agrochemically acceptable salt, stereoisomer, or N-oxide thereof.

2. The compound of formula (I) according to claim 1, wherein R¹ is methyl; and wherein R² is selected from hydrogen or methyl.

3. The compound of formula (I) according to claim 1, wherein R³ is hydrogen; and R⁴ is selected from hydrogen or methyl.

4. The compound of formula (I) according to claim 1, wherein R⁵ and R⁶ are independently selected from hydrogen or methyl.

5. The compound of formula (I) according to claim 1, wherein R⁷ is hydrogen, C₁-C₄alkyl, or C₃-C₆-cycloalkyl.

6. The compound of formula (I) according to claim 1, wherein A¹ is selected from CR^{12a} or N; A² is selected from CR^{13a} or N; A³ is selected from CR^{14a} or N; wherein at least two of A¹, A² and A³ are selected from N; and wherein R^{12a}, R^{13a}, and R^{14a} are hydrogen.

7. The compound of formula (I) according to claim 1, wherein B¹ is CR¹⁰; and B² is CR¹¹.

8. The compound of formula (I) according to claim 1, wherein
R⁸ and R⁹ are independently selected from hydrogen, halogen, cyano, C₁-C₄ alkyl, or C₁-C₄ alkoxy; and
R¹⁰ and R¹¹ are independently selected from hydrogen, halogen, C₁-C₃ alkyl, or C₁-C₄ alkoxy.

9. The compound of formula (I) according to claim 8, wherein R¹⁰ and R¹¹ are hydrogen.

10. A compound of formula (I) according to claim 1, wherein the compound of formula (I) is a compound of formula (II): 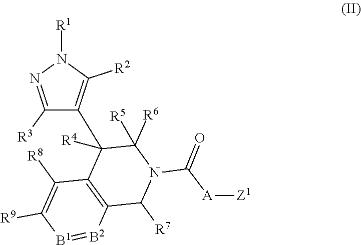
wherein R¹, R², R³, R⁴, R⁵, R⁶, R⁷, R⁸, R⁹, B¹ and B² and Z¹ are as defined for the compounds of formula (I) according to claim 1, and A is selected from: 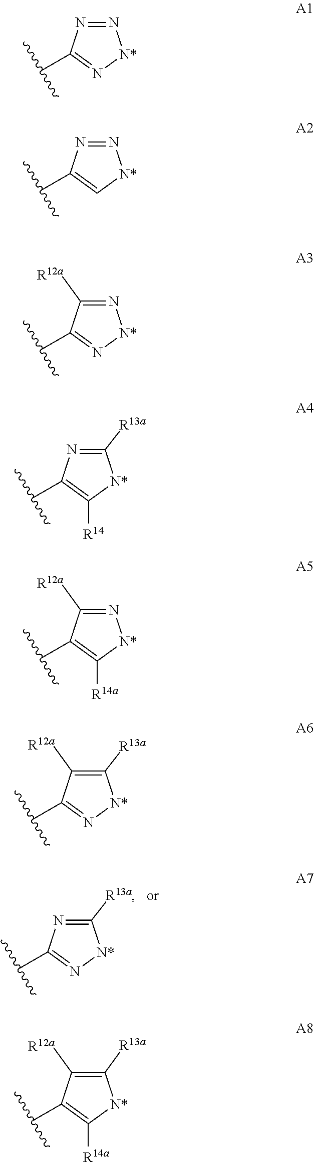
wherein 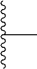
indicates the bond to the C(═O) group, and the star (*) the nitrogen with the bond to the Z¹ group, and wherein R^{12a}, R^{13a} and R^{14a} are independently selected from hydrogen, halogen, methyl, cyclopropyl, or trifluoromethyl.

11. The compound of formula (II) according to claim 10, wherein A is selected from. 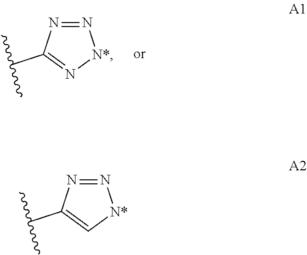
wherein 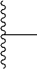
indicates the bond to the C(═O) group, and the star (*) the nitrogen with the bond to the Z¹ group.

12. The compound of formula (I) according to claim 1, wherein Z¹ is selected from 1-methylpyrazol-4-yl, 2,4,6-trifluorophenyl, 3,5-difluoro-2-pyridyl, 2,4-difluorophenyl, 2-fluorophenyl, 2-furyl, 2-methylphenyl, 2-thienyl, 3,4-difluorophenyl, 3-chlorophenyl, 3-thienyl, 4-fluoro-2-methoxy-phenyl, 4-fluorophenyl, cyclobutyl, cyclohexyl, cyclopentyl, or phenyl.

13. An agrochemical composition comprising a fungicidally effective amount of a compound of formula (I) as defined in claim 1.

14. A method of controlling or preventing infestation of useful plants by phytopathogenic microorganisms, wherein a fungicidally effective amount of a compound of formula (I) as defined in claim 1, or a composition comprising the compound of formula (I), is applied to the plants, to parts thereof or the locus thereof.

15. Use of a compound according to claim 1 as a fungicide.
